(19) United States
(12) Patent Application Publication
(10) Pub. No.: US 2025/0346943 A1
(43) Pub. Date: Nov. 13, 2025

(54) DEGRADER COMPOUNDS OF QSOX1 mRNA

(71) Applicant: UNIVERSITY OF FLORIDA RESEARCH FOUNDATION, INCORPORATED, Gainesville, FL (US)

(72) Inventor: Matthew D. Disney, Gainesville, FL (US)

(73) Assignee: UNIVERSITY OF FLORIDA RESEARCH FOUNDATION, INCORPORATED, Gainesville, FL (US)

(21) Appl. No.: 18/869,077

(22) PCT Filed: May 26, 2023
(86) PCT No.: PCT/US2023/023657

§ 371 Date: Nov. 25, 2024

Related U.S. Application Data
(60) Provisional application No. 63/346,793, filed on May 27, 2022.
(60) Provisional application No. 63/349,973, filed on Jun. 7, 2022.

(51) Int. Cl. C12Q 1/6806; C07D 229/02; C07D 333/38; C07D 401/12; C07D 401/14; C07D 403/12; C07D 405/12; C07D 413/12; C07D 417/12; C07D 417/14; C07D 453/02; C07D 471/04; C07D 473/18; C07D 487/04; C07D 495/04; C07H 17/02; C12N 15/10

(52) U.S. Cl. CPC C12Q 1/6806; C07D 229/02; C07D 333/38; C07D 401/12; C07D 401/14; C07D 403/12; C07D 405/12; C07D 413/12; C07D 417/12; C07D 417/14; C07D 453/02; C07D 471/04; C07D 473/18; C07D 487/04; C07D 495/04; C07H 17/02; C12N 15/1093

(57) ABSTRACT

The present disclosure provides compounds of the formulae herein (e.g., Formulae (I) or (II)), and pharmaceutically acceptable salts thereof, which are degrader compounds of Quiescin Sulfhydryl Oxidase 1 (QSOX1) mRNA. The present disclosure also provides pharmaceutical compositions and kits comprising the compounds, or pharmaceutically acceptable salts thereof, and methods of treating or preventing diseases. Related compounds and methods useful in probing RNA targets and studying molecular recognition patterns between RNA and ligands are described.

### GOVERNMENT SUPPORT

This invention was made with government support under grant number R01 CA249180, awarded by the National Institutes of Health. The government has certain rights in the invention.

### BACKGROUND OF THE INVENTION

The importance of RNA in all aspects of biology is well-established, with its function dependent on its structure.^{1, 2} One way to model RNA structure is to use a free energy minimization restrained by chemical probing data.^{3,4} Alternatively, defining the binding sites of small molecules could allow for direct inference of RNA structure in cells and could be particularly important if binding stabilizes dynamic structures, enhancing their detectability. Molecular recognition could be mediated by the RNA's structure, an RNA-protein interface, or other factors.^{5, 6} Covalent chemistry has been used to define RNAs bound by small molecules and their target sites in cells by using Chemical Cross-Linking and Isolation by Pull-down (Chem-CLIP).⁷⁻⁹

### SUMMARY OF THE INVENTION

Covalent chemistry and RNA profiling in live mammalian cells can define the RNA targets of low molecular weight small molecules. As such, this approach is the RNA parallel of profiling small molecules for protein and DNA targets.¹⁰⁻¹³

Accordingly, in one aspect, the present disclosure provides methods of transcriptome-wide mapping of RNA binding sites in a cell, comprising (a) providing purified total RNA from a cell; (b) combining the purified total RNA with a compound comprising a diazirine moiety and an alkyne moiety to form a mixture; (c) irradiating the mixture; (d) treating the irradiated mixture with a fluorescent dye comprising an azide moiety or an agarose comprising an azide moiety under conditions wherein triazolyl-bound RNA is formed; and (e) evaluating the resulting triazolyl-bound RNA to identify a target RNA and binding site within the target RNA. In certain embodiments, evaluating the resulting triazolyl-bound RNA is by gel electrophoresis or fluorescence imaging. In certain embodiments, the cell is a cancer cell (e.g., a breast cancer cell (e.g., an MDA-MB-231 triple negative breast cancer cell)). In certain embodiments, the irradiation is with ultraviolet light. In certain embodiments, evaluating the resulting triazolyl-bound RNA comprises identifying enrichment of the target RNA. In certain embodiments, the methods are selective for enrichment of the target RNA compared to DNA or proteins. In certain embodiments, the methods further comprise using a control compound comprising a diazirine moiety and an alkyne moiety, wherein the control compound does not comprise a moiety capable of binding RNA, to identify a control target that non-specifically reacts with the diazirine moiety.

In another aspect, the present disclosure provides methods of transcriptome-wide mapping of RNA binding sites in a cell, comprising (a) providing a cell; (b) treating the cell with a compound comprising a diazirine moiety and an alkyne moiety to form a mixture; (c) irradiating the mixture; (d) treating the irradiated mixture with an agarose comprising an azide moiety under conditions wherein triazolyl-bound RNA is formed; (e) harvesting from the mixture total RNA comprising the triazolyl-bound RNA; (f) fragmenting the total RNA; (g) performing pull-down of the triazolyl-bound RNA; and (h) evaluating the resulting triazolyl-bound RNA to identify the target RNA and binding site within the target RNA. In certain embodiments, fragmenting the total RNA comprises random fragmentation. In certain embodiments, evaluating the resulting triazolyl-bound RNA is by gel electrophoresis. In certain embodiments, the cell is a cancer cell (e.g., a breast cancer cell (e.g., an MDA-MB-231 triple negative breast cancer cell)). In certain embodiments, the irradiation is with ultraviolet light. In certain embodiments, evaluating the resulting triazolyl-bound RNA comprises identifying enrichment of the target RNA. In certain embodiments, the methods are selective for enrichment of the target RNA compared to DNA or proteins. In certain embodiments, the methods further comprise using a control compound comprising a diazirine moiety and an alkyne moiety, wherein the control compound does not comprise a moiety capable of binding RNA, to identify a control target that non-specifically reacts with the diazirine moiety.

In another aspect, the present disclosure provides methods of making modified ribonucleic acids, or pharmaceutically acceptable salts thereof, comprising reacting ribonucleic acids with compounds of Formula (I):

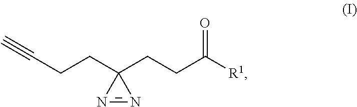

or pharmaceutically acceptable salts thereof, wherein R¹ is as defined herein.

In another aspect, the present disclosure provides modified ribonucleic acids, or pharmaceutically acceptable salts thereof, made by reacting ribonucleic acids with compounds of Formula (I):

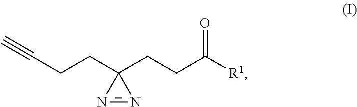

or pharmaceutically acceptable salts thereof, wherein R¹ is as defined herein.

In another aspect, the present disclosure provides compounds of Formula (I):

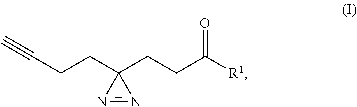

or pharmaceutically acceptable salts thereof, wherein R¹ is as defined herein.

In another aspect, the present disclosure provides compounds of Formula (II):

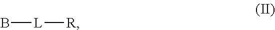

or pharmaceutically acceptable salts thereof, wherein B, L, and R are as defined herein.

In another aspect, the present disclosure provides pharmaceutical compositions comprising a compound disclosed herein. In some embodiments, the pharmaceutical composition comprises an excipient.

In another aspect, the present disclosure provides methods of binding RNase L in a subject in need thereof or in a cell, tissue, or biological sample in a subject in need thereof or in a cell, tissue, or biological sample, comprising administering to the subject in need thereof or contacting the cell, tissue, or biological sample with an effective amount of a provided compound or pharmaceutical composition. In certain embodiments, the cell, tissue, or biological sample is in vivo. In certain embodiments, the cell, tissue, or biological sample is in vitro. In certain embodiments, binding RNase L comprises activating RNase L. In certain embodiments, binding RNase L comprises inducing RNase L dimerization.

In another aspect, the present disclosure provides methods of modulating QSOX1 mRNA in a subject in need thereof or in a cell, tissue, or biological sample in a subject in need thereof or in a cell, tissue, or biological sample, comprising administering to the subject in need thereof or contacting the cell, tissue, or biological sample with an effective amount of a provided compound or pharmaceutical composition. In certain embodiments, the cell, tissue, or biological sample is in vivo. In certain embodiments, the cell, tissue, or biological sample is in vitro. In certain embodiments, the QSOX1 mRNA is QSOX1-a mRNA.

In another aspect, the present disclosure provides methods of degrading a QSOX1 mRNA isoform in a subject in need thereof or in a cell, tissue, or biological sample in a subject in need thereof or in a cell, tissue, or biological sample, comprising administering to the subject in need thereof or contacting the cell, tissue, or biological sample with an effective amount of a provided compound or pharmaceutical composition. In certain embodiments, the cell, tissue, or biological sample is in vivo. In certain embodiments, the cell, tissue, or biological sample is in vitro. In certain embodiments, the QSOX1 mRNA isoform is QSOX1-a.

In another aspect, the present disclosure provides methods of inhibiting cell proliferation or promoting apoptosis in a subject in need thereof or in a cell, tissue, or biological sample in a subject in need thereof or in a cell, tissue, or biological sample, comprising administering to the subject in need thereof or contacting the cell, tissue, or biological sample with an effective amount of a provided compound or pharmaceutical composition. In certain embodiments, the cell, tissue, or biological sample is in vivo. In certain embodiments, the cell, tissue, or biological sample is in vitro. In certain embodiments, the method further comprises reducing an amount of QSOX1 protein. In certain embodiments, the QSOX1 protein is QSOX1-a.

In another aspect, the present disclosure provides methods of treating or preventing a disease in a subject in need thereof, comprising administering to the subject in need thereof a provided compound or pharmaceutical composition. In certain embodiments, the disease is associated with QSOX1 mRNA (e.g., breast cancer (e.g., triple negative breast cancer)). In certain embodiments, the disease is breast cancer (e.g., triple negative breast cancer). In certain embodiments, the disease is triple negative breast cancer.

In another aspect, the present disclosure provides methods of preparing compounds of Formulae (II-a) or (II-b):

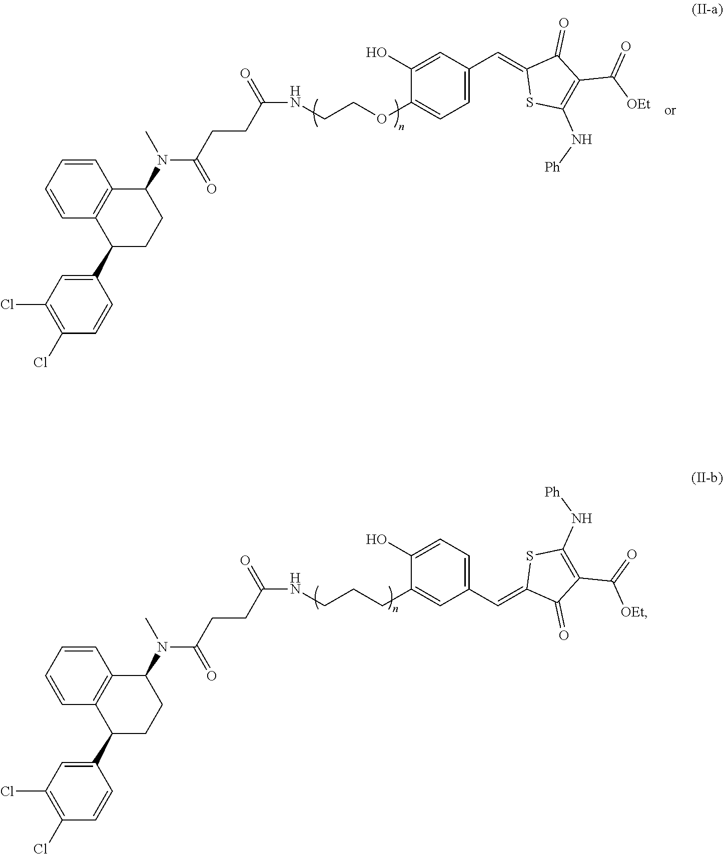

or pharmaceutically acceptable salts prodrugs thereof, wherein n is as defined herein.

In another aspect, the present disclosure provides kits comprising a provided compound or pharmaceutical composition disclosed herein and instructions for its use.

It should be appreciated that the foregoing concepts, and the additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying drawings.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIGS. 1A-1B show that in vitro screening identifies low molecular weight ligands that preferentially bind RNA. FIG. 1A shows structures of compounds identified as hits with fluorescence at least 3-fold above the background. FIG. 1B shows a schematic depiction of in vitro screening of low molecular weight compounds binding to total human RNA from MDA-MB-231 TNBC cells.

FIGS. 2A-2D show that Chem-CLIP-Map-Seq profiles the targets of compounds across the human transcriptome in cellulis. FIG. 2A shows a schematic depiction of Chem-CLIP-Seq workflow. FIG. 2B shows a heatmap representation of genes enriched by each compound. FIG. 2C shows comparisons of genes enriched by each compound. FIG. 2D shows genes that are significantly enriched by F1 identified by Chem-CLIP-Seq in MDA-MB-231 cells.

FIGS. 3A-3C show that the F1 binding site is mapped to QSOX1 5′ UTR and subsequently converted to an RNA degrader. FIG. 3A shows RNA-seq tracks showing regions of QSOX1 transcript enriched by F1 in cells. FIG. 3B shows the RNA sequence near mapped F1 crosslinking site. FIG. 3C shows the structures of F1-RIBOTAC that recruits RNase L, and F1-CTRL that is >20-fold less active in recruiting RNase L.

FIGS. 4A-4C show that F1-RIBOTAC decreases QSOX1 mRNA and protein levels. FIG. 4A shows the effect of F1-Amide and F1-RIBOTAC on QSOX1 mRNA levels (n=4). FIG. 4B shows the effect of F1-RIBOTAC on QSOX1 mRNA levels in RNase L knockout MDA-MB-231 cells and control CRISPR cells (n=7). FIG. 4C shows F1-RIBOTAC inhibits the expression of QSOX1 protein (n=4). *p<0.05, **p<0.01, ****p<0.0001.

FIGS. 5A-5C show that F1-RIBOTAC inhibits invasion and proliferation in MDA-MB-231 cells. FIG. 5A shows images of invasive MDA-MB-231 with or without treatment. FIG. 5B shows quantification of invasive cells (n=3) FIG. 5C shows the effect of F1-RIBOTAC and F1-Amide on the proliferation of MDA-MB-231 cells after 48 h treatment (n=6). **p<0.01, ***p<0.001, ****p<0.0001.

FIG. 6 shows chemical structures of 34 compounds synthesized for in vitro screening. The number shown is the average Tanimoto score calculated for each structure compared to all known RNA binders.^{1, 2} F1 to F6 were previously reported to bind RNA from an in vitro screening.³ A Tanimoto score <0.7 is typically considered as chemically dissimilar. The core structures highlighted represent novel chemotypes that are not present in previously known RNA binders. This library was constructed based on the by UMAP (Uniform Manifold Approximation and Projection) analysis (FIG. 7) to identify molecules that are similar to known RNA binders in a broad sense, as well as including novel chemotypes that are not previously reported to bind RNA in cells and commercial availability (F24/F25).

FIGS. 7A-7B show a comparison of screened compounds with known small molecules that bind RNA. FIG. 7A shows the panel of 34 structurally diverse compounds are clustered with ˜80% of all known RNA binding small molecules^{1, 2} as determined by UMAP (Uniform Manifold Approximation and Projection) analysis based on Morgan fingerprints⁴ of structural similarities. FIG. 7B shows Tanimoto scores of hit compounds (F1 to F6) compared with each known RNA-binding small molecules suggest that they are overall dissimilar to known RNA binders (Tanimoto score <0.7). Among these six hits, F1 is the most dissimilar compound with average Tanimoto score of only 0.27±0.09. The other five hits are also dissimilar to most RNA-binding compounds, although the range of values for F2-F5 extend over the 0.7 cut-off. FIG. 7C shows all six hits showed lower polar surface area (PSA) than average known RNA binders, and F1 shows the lowest PSA. FIG. 7D shows that F1 shows the highest atomic log P (AlopP) compared to other hits and the average known RNA binders.

FIG. 8 shows representative images of in vitro Chem-CLIP screening of compounds cross-linked with purified total RNA from MDA-MB-231 cells. The general reactivity of compounds was studied using in vitro Chem-CLIP by cross-linking the compound to purified total RNA from MDA-MB-231 cells. Cross-linking was then visualized by a click reaction with azide-functionalized TAMRA (tetramethylrhodamine) dye, and the approximate sizes of the cross-linked RNA analyzed by agarose gel electrophoresis. After imaging TAMRA fluorescence, the gel was stained with SYBR Green to visualize total RNA. A fragment was deemed a hit if the resulting TAMRA signal was at least 3-fold above the background. “Control” indicates incubation of total RNA with the control diazirine probe shown at the top of the figure.

FIGS. 9A-9B show a comparison of F1-F6 to molecules previously reported chemoproteomic studies that use fully functionalized fragments.^{5, 6} FIG. 9A shows the chemical structures of fully functionalized fragments (FFFs) previously reported in chemoproteomic studies. FIG. 9B shows a comparison of physicochemical properties of F1-F6 to those shown in FIG. 9A.

FIG. 10 shows transcripts enriched by F2-F6 and the control diazirine probe using Chem-CLIP-seq in MDA-MB-231 cells. Genrich uses a null model with a log-normal distribution to calculate p values by comparing sequencing runs of the same RNA sample before and after pull-down. A minimum read count of 5 and fold enrichment of 1.5 were applied to filter out low-confidence peaks from the RNA-seq analysis. F2 enriched 166 transcripts, including 52 transcripts (31%) that were also enriched by the control probe. F3 enriched 163 transcripts, including 53 transcripts (32%) that were also enriched by the control probe. F4 enriched 173 transcripts, including 45 transcripts (43%) that were also enriched by the control probe. F5 enriched 92 transcripts, including 57 transcripts (62%) that were also enriched by the control probe. F6 enriched 127 transcripts, including 55 transcripts (43%) that were also enriched by the control probe. The control probe enriched 102 transcripts.

FIG. 11 shows transcripts enriched by F1-F6 using Chem-CLIP-seq in MDA-MB-231 cells after excluding genes overlapping with the control diazirine probe.

FIG. 12 shows the distribution of binding sites within targeted transcripts by each small molecule. Regions are classified as 5′ or 3′ untranslated regions (UTRs), coding regions (CDS), introns, or non-coding RNAs.

FIG. 13 shows analysis of the structures of the regions pulled down by each small molecule by ScanFold. ScanFold predicts the secondary structure of an RNA by using a scanning window. That is, the transcript is folded in 120-nucleotide increments, moving down the sequence in 1 bp increments (step size=1). The stability of the resultant structures in each window is compared to the average stability of 100 random sequences of the same nucleotide composition. Thus, this program identifies unusually stable structures within an RNA. A Z-score <−1 indicates a structure that is 1 standard deviation more stable than a random sequence (1s) while a Z-score <−2 indicates a structure that is 2 s.

FIGS. 14A-14D show evaluation of RNA, DNA, and proteins cross-linked by F1 in MDA-MB-231 cells. MDA-MB-231 cells were treated with 20 mM F1 or control probe lacking an RNA-binding module (“Control”) for 16 h followed by UV irradiation and isolation of total RNA, DNA, and protein from the same batch of cells. All samples were clicked with TAMRA azide and analyzed by gel electrophoresis. TAMRA imaging was used to identify the crosslinked RNA (FIG. 14A), DNA (FIG. 14B), and proteins (FIG. 14C). SYBR green staining was used visualize total RNA or DNA, and Coomassie staining was used to visualize total proteins. FIG. 14D shows quantification (n=2), where the total signal intensity in the “Control” lane was set to 1, and the TAMRA intensity in the F1 lanes was normalized accordingly. *p<0.05 as measured by a Student's t test. Error bars are reported as SD.

FIG. 15 shows validation of primers used in qPCR experiments. Melting curves of qPCR products are shown on the left, and linear correlations between the qPCR Ct values and the fold of input cDNA dilutions are shown on the right. Each experiment was performed with three biological replicates. Error bars are reported as SD.

FIG. 16 shows gel visualization of qPCR products. A single band product with expected size was observed for all primer sets with input cDNA templates. No band was observed from qPCR amplification in the absence of cDNA template.

FIGS. 17A-17B show concentration- and time-dependence of the pull-down of QSOX1 mRNA by F1. FIG. 17A shows enrichment of QSOX1 mRNA levels by F1 at varying concentrations in MDA-MB-231 cells, as measured by RT-qPCR (n=3). FIG. 17B shows time course of F1 pull-down of QSOX1 mRNA in MDA-MB-231 cells, as measured by RT-qPCR (n=3). *p<0.05, **p<0.01, ***p<0.001 as measured by a Student's t test. Error bars are reported as SD.

FIG. 18 shows structural differences in the 5′ UTR of the two isoforms of QSOX1. The mRNA transcript encoding QSOX1-a (75 kDa) contains the 5′ UTR sequence that folds into the hairpin (shown on the left) targeted by F1. The arrow indicates the U-bulge engaged by F1. The mRNA encoding QSOX1-b (60 kDa), however, has a truncated 5′ UTR sequence that can no longer fold into the same hairpin structure.

FIGS. 19A-19B show the predicted structure of SOSTM1 binding site for F1. FIG. 19A shows the secondary structure of enriched sequence as predicted by using ScanFold. The arrow indicates the mapped cross-linking site by F1 in cells. FIG. 19B shows the sequencing tracks showing the enrichment of SQSTM1 mRNA by F1 in cells. Top: the ratio of sequencing reads after vs. before the pulldown. The range reported in the upper left corner indicates the scale of the y-axis, reported as Fold Enrichment; Middle: raw sequencing track before pulldown. The range reported in the upper left corner indicates the scale of the y-axis, reported as Read Count); Bottom: raw sequencing track after pulldown. The range shown in the upper left corner indicates the scale of the y-axis, reported as Read Count.

FIG. 20 shows in vitro binding of F1 and F1 derivatives to the QSOX1 5′UTR hairpin. Cy5-labeled RNA constructs were used to measure changes in fluorescence polarization (Ex. 640 nm, Em. 680 nm) upon incubating with an increasing concentration of small molecule. Each experiment was performed with two independent replicates, and error bars are reported as SD.

FIG. 21 shows in vitro melting experiments with WT and mutant model of the QSOX1-a hairpin structure. Top: Addition of F1-Amide significantly increased the Tₘ of WT model RNA (from 51.8 to 53.7° C.) and decreased the ΔG_{37° C.} from −2.7 to −3.0 kcal/mol (n=3). Bottom: No significant effect on the Tₘ or ΔG_{37° C.} was observed upon additional of F1-Amide to the mutant RNA, which the targeted U-bulge was mutated to the U/A base pair (n=3) **p<0.01 as measured by a Student's t test. Error bars are reported as SD.

FIGS. 22A-22B show target validation by in vitro Chem-CLIP with F1 and Cy5-labeled RNA constructs. FIG. 22A shows F1 significantly pulled down wild type (WT) QSOX1 5′ UTR RNA hairpin but not the base paired (BP) mutant RNA lacking the U-bulge (n=3). FIG. 22B shows co-incubation of the QSOX1-a hairpin with an increasing concentration of F1-RIBOTAC and a constant concentration of F1 (20 mM) dose-dependently ablates the cross-linking of F1 to the RNA in vitro (n=3). **p<0.01, ***p<0.001 as measured by a Student's t test. Error bars are reported as SD.

FIGS. 23A-23C show the effect of F1-CTRL and F1-Amide on QSOX1 mRNA and protein levels in MDA-MB-231 cells. FIG. 23A shows the effect of F1-CTRL on QSOX1 mRNA levels in MDA-MB-231 cells, as measured by RT-qPCR (n=3). FIG. 23B shows the effect of F1-Amide on QSOX1 protein levels in MDA-MB-231 cells, measured by Western blot (n=5 for vehicle; n=4 for F1-Amide). FIG. 23C shows the effect of F1-Control and an antisense oligonucleotide (ASO) on QSOX1 protein levels in MDA-MB-231 cells, measured by Western blot (n=4 for vehicle; n=3 for ASO, F1-CTRL, and Scrambled ASO). *p<0.05, ***p<0.001, ****p<0.0001 as measured by a Student's t test. Error bars are reported as SD.

FIGS. 24A-24C show the effect of F1-Amide and F1-RIBOTAC on SQSTM1 mRNA and protein levels. FIG. 24A shows the effect of F1-RIBOTAC on SOSTM1 mRNA levels upon treatment of MDA-MB-231 cells for 48 h (n=4), as measured by RT-qPCR. FIG. 24B shows representative Western blot image measuring the abundance of SOSTM1 protein levels in MDA-MB-231 cells treated with F1-Amide (20 μM) and F1-RIBOTAC (10 μM) for 48 h. FIG. 24C shows quantification of the abundance of SOSTM1 protein levels normalized to GAPDH protein levels (n=4).

FIG. 25 shows the effect of F1-Amide and F1-CTRL on invasion phenotype of MDA-MB-231 cells. Top: representative microscopic images showing invasive cells with the indicated treatment. Bottom: quantification of the number of invasive cells upon treatment as indicated for 48 h (n=3 biological replicates).

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

Definitions of specific functional groups and chemical terms are described in more detail below. The chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc, New York, 2013, Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ of Notre Dame Press, Notre Dame, IN 1972). The invention additionally encompasses compounds as individual isomers substantially free of other isomers, and alternatively, as mixtures of various isomers. The term “isomers” is intended to include diastereoisomers, enantiomers, regioisomers, structural isomers, rotational isomers, tautomers, and the like. All such isomers of such compounds herein are expressly included in the present invention.

When a range of values (“range”) is listed, it encompasses each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or (—Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., CH₂F, —CHF₂, —CF₃, —CH₂CH₂F, —CH₂CHF₂, —CH₂CF₃, or benzyl (Bn)).

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl and refers to an alkyl group wherein all of the hydrogen atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, the haloalkyl moiety has 1 to 10 carbon atoms (“C₁₋₁₀ haloalkyl”). In some embodiments, the haloalkyl moiety has 1 to 9 carbon atoms (“C₁₋₉ haloalkyl”). In some embodiments, the haloalkyl moiety has 1 to 8 carbon atoms (“C₁₋₈ haloalkyl”). In some embodiments, the haloalkyl moiety has 1 to 7 carbon atoms (“C₁₋₇ haloalkyl”). In some embodiments, the haloalkyl moiety has 1 to 6 carbon atoms (“C₁₋₆ haloalkyl”). In some embodiments, the haloalkyl moiety has 1 to 5 carbon atoms (“C₁₋₅ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are independently replaced with fluoro to provide a “perfluoroalkyl” group. In some embodiments, all of the haloalkyl hydrogen atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉) alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The term “alkenyl” refers to a radical of a straight-chain or branched hydrocarbon group having from 2 to 20 carbon atoms and one or more carbon-carbon double bonds (e.g., 1, 2, 3, or 4 double bonds). In some embodiments, an alkenyl group has 2 to 20 carbon atoms (“C₂₋₂₀ alkenyl”). In some embodiments, an alkenyl group has 2 to 12 carbon atoms (“C₂₋₁₂ alkenyl”). In some embodiments, an alkenyl group has 2 to 11 carbon atoms (“C₂₋₁₁ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₈ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₂₀ alkenyl. In certain embodiments, the alkenyl group is a substituted C₂₋₂₀ alkenyl. In an alkenyl group, a C═C double bond for which the stereochemistry is not specified (e.g., —CH═CHCH₃ or

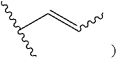

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 2 to 20 carbon atoms, at least one double bond, and 1 or more heteroatoms within the parent chain (“heteroC₂₋₂₀ alkenyl”). In certain embodiments, a heteroalkenyl group refers to a group having from 2 to 12 carbon atoms, at least one double bond, and 1 or more heteroatoms within the parent chain (“heteroC₂₋₁₂ alkenyl”). In certain embodiments, a heteroalkenyl group refers to a group having from 2 to 11 carbon atoms, at least one double bond, and 1 or more heteroatoms within the parent chain (“heteroC₂₋₁₁ alkenyl”). In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 carbon atoms, at least one double bond, and 1 heteroatom within the parent chain (“heteroC₂ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₂₀ alkenyl. In certain embodiments, the heteroalkenyl group is a substituted heteroC₂₋₂₀ alkenyl.

The term “alkynyl” refers to a radical of a straight-chain or branched hydrocarbon group having from 2 to 20 carbon atoms and one or more carbon-carbon triple bonds (e.g., 1, 2, 3, or 4 triple bonds) (“C₁₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉) alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂-20 alkynyl. In certain embodiments, the alkynyl group is a substituted C₂₋₂₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 2 to 20 carbon atoms, at least one triple bond, and 1 or more heteroatoms within the parent chain (“heteroC₂₋₂₀ alkynyl”). In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and 1 or 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 carbon atoms, at least one triple bond, and 1 heteroatom within the parent chain (“heteroC₂ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₂₀ alkynyl. In certain embodiments, the heteroalkynyl group is a substituted heteroC₂₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈) Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” or “fully saturated” refers to a moiety that does not contain a double or triple bond, e.g., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not limited in any manner by the exemplary substituents described herein.

Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR BC(═NR^{bb})N(R^{bb})₂, —C(═O)NR^{bb}SO₂R^{aa}, —NR^{bb}SO₂R^{aa}, —SON(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃, —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{cc})₂, —OP(═O)(OR^{cc})₂, —P(═O))(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})+, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
wherein:
each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —COR^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R™)₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O))(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀alkyl, heteroC₁₋₁₀alkenyl, heteroC₁₋₁₀alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents are joined to form ═O or ═S; wherein X⁻ is a counterion;
each instance of R^{ee} is, independently, selected from C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₃⁺X⁻, —NH₂(C₁₋₆ alkyl)⁺X⁻, —NH₃⁻X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl) C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃C(═S)N(C₁₋₆ alkyl)₂, —C(═S)NH(C₁₋₆ alkyl), —C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl; or two geminal R^{gg} substituents can be joined to form ═O or ═S; and
each X⁻ is a counterion.

In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, each carbon atom substituent is independently halogen, substituted (e.g., substituted with one or more halogen) or unsubstituted C₁₋₁₀ alkyl, —OR^{aa}, —SR^{aa}, —N(R^{bb})₂, —CN, —SCN, —NO₂, —C(═O)R^{aa}, —CO₂R^{aa}, —C(═O))N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, CN, —SCN, or —NO₂. In certain embodiments, each carbon atom substituent is independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

In certain embodiments, the molecular weight of a carbon atom substituent is lower than 250, lower than 200, lower than 150, lower than 100, or lower than 50 g/mol. In certain embodiments, a carbon atom substituent consists of carbon, hydrogen, fluorine, chlorine, bromine, iodine, oxygen, sulfur, nitrogen, and/or silicon atoms. In certain embodiments, a carbon atom substituent consists of carbon, hydrogen, fluorine, chlorine, bromine, iodine, oxygen, sulfur, and/or nitrogen atoms. In certain embodiments, a carbon atom substituent consists of carbon, hydrogen, fluorine, chlorine, bromine, and/or iodine atoms. In certain embodiments, a carbon atom substituent consists of carbon, hydrogen, fluorine, and/or chlorine atoms.

The term “halo” or “halogen” refers to fluorine (fluoro, —F), chlorine (chloro, —C₁), bromine (bromo, —Br), or iodine (iodo, —I).

The term “hydroxyl” or “hydroxy” refers to the group —OH. The term “substituted hydroxyl” or “substituted hydroxyl.” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

The term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═S)OR^{aa}, —SC(═S)N(R^{bb})₂, —SC(═O)SR^{aa}, —SC(═O)OR^{aa}, —SC(═O)N(R^{bb})₂, and —SC(═O)R^{aa}, wherein R^{aa} and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O))R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, NR^{bb} C(═O)R^{aa}, —NR^{bb}CO₂R^{aa}, —NRDC(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb}); and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic, cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers to a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O) NR^{bb}SO₂R^{aa}, —C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

The term “silyl” refers to the group —Si(R^{aa})₃, wherein R^{aa} is as defined herein.

The term “phosphino” refers to the group —P(R^{cc})₂, wherein R^{aa} is as defined herein.

The term “phosphono” refers to the group —(P═O)(OR^{cc})₂, wherein R^{aa} and R^{cc} are as defined herein.

The term “phosphoramido” refers to the group —O(P═O)(N(R^{bb})₂)₂, wherein each R^{bb} is as defined herein.

The term “oxo” refers to the group —O, and the term “thiooxo” refers to the group ═S.

Nitrogen atoms can be substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SOR^{cc}, —SO₂OR^{cc}, —SOR^{aa}C(═S)N(R^{cc})₂, —C(═O)SR^{cc}C(═S)SR^{cc}, —P(═O)(OR^{cc})₂, —P(═O)(R^{aa})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each nitrogen atom substituent is independently substituted (e.g., substituted with one or more halogen) or unsubstituted C₁₋₆ alkyl or a nitrogen protecting group.

In certain embodiments, the substituent present on the nitrogen atom is a nitrogen protecting group (also referred to herein as an “amino protecting group”). Nitrogen protecting groups include —OH, OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, in certain embodiments, at least one nitrogen protecting group is an amide group (e.g., a moiety that include the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group consisting of formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivatives, benzamide, p-phenylbenzamide, o-nitophenylacetamide, o-nitrophenoxyacetamide, acetoacetamide, (N′-dithiobenzyloxyacylamino) acetamide, 3-(p-hydroxyphenyl) propanamide, 3-(o)-nitrophenyl) propanamide, 2-methyl-2-(o-nitrophenoxy) propanamide, 2-methyl-2-(o-phenylazophenoxy) propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

In certain embodiments, at least one nitrogen protecting group is a carbamate group (e.g., a moiety that includes the nitrogen atom to which the nitrogen protecting groups (e.g., —C(═O)OR^{aa}) is directly attached). In certain such embodiments, each nitrogen protecting group, together with the nitrogen atom to which the nitrogen protecting group is attached, is independently selected from the group consisting of methyl carbamate, ethyl carbamate, 9-fluorenylmethyl carbamate (Fmoc), 9-(2-sulfo) fluorenylmethyl carbamate, 9-(2,7-dibromo) fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), I-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl (o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido) propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

In certain embodiments, at least one nitrogen protecting group is a sulfonamide group (e.g., a moiety that include the nitrogen atom to which the nitrogen protecting groups (e.g., —S(═O))₂R^{aa}) is directly attached). In certain such embodiments, each nitrogen protecting group, together with the nitrogen atom to which the nitrogen protecting group is attached, is independently selected from the group consisting of p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In certain embodiments, each nitrogen protecting group, together with the nitrogen atom to which the nitrogen protecting group is attached, is independently selected from the group consisting of phenothiazinyl-(10)-acyl derivatives, N′-p-toluenesulfonylaminoacyl derivatives, N′-phenylaminothioacyl derivatives, N-benzoylphenylalanyl derivatives, N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl) ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di (4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethylencamine, N-ferrocenylmethylamino (Fcm), N-2-picolylamino N′-oxide, N-1, 1-dimethylthiomethyleneamine, N-benzylideneamine, N-p-methoxybenzylideneamine, N-diphenylmethyleneamine, N-[(2-pyridyl) mesityl]methyleneamine, N—(N′,N′-dimethylaminomethylene)amine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivatives, N-diphenylborinic acid derivatives, N-[phenyl(pentaacylchromium- or tungsten) acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys) In some embodiments, two instances of a nitrogen protecting group together with the nitrogen atoms to which the nitrogen protecting groups are attached are N,N′-isopropylidenediamine.

In certain embodiments, at least one nitrogen protecting group is Bn, Boc, Cbz, Fmoc, trifluoroacetyl, triphenylmethyl, acetyl, or Ts.

In certain embodiments, each oxygen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or an oxygen protecting group. In certain embodiments, each oxygen atom substituents is independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each oxygen atom substituent is independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(O)R^{aa}, —COR^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O))(R^{aa})₂, —P(═O))(OR^{cc})₂, and —P(═O))(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, each oxygen protecting group, together with the oxygen atom to which the oxygen protecting group is attached, is selected from the group consisting of methyl, methoxymethyl (MOM), methylthiomethyl (MTM), t-butylthiomethyl, (phenyldimethylsilyl) methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl) ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, 1-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p)-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 4,4′-Dimethoxy-3″′-[N-(imidazolylmethyl)]trityl Ether (IDTr-OR), 4,4′-Dimethoxy-3″′-[N-(imidazolylethyl) carbamoyl]trityl Ether (IETr-OR), 1,1-bis(4-methoxyphenyl)-1′-pyrenylmethyl, 9-anthryl, 9-(9-phenyl) xanthenyl, 9-(9-phenyl-10-oxo) anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, 1-butyldimethylsilyl (TBDMS), 1-butyldiphenylsilyl (TBDPS), tribenzylsilyl, tri-p-xylylsilyl, triphenylsilyl, diphenylmethylsilyl (DPMS), 1-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio) pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio)ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate (MTMEC-OR), 4-(methylthiomethoxy) butyrate, 2-(methylthiomethoxymethyl)benzoate, 2,6-dichloro-4-methylphenoxyacetate, 2,6-dichloro-4-(1,1,3,3-tetramethylbutyl) phenoxyacetate, 2,4-bis(1,1-dimethylpropyl) phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, at least one oxygen protecting group is silyl, TBDPS, TBDMS, TIPS, TES, TMS, MOM, THP, t-Bu, Bn, allyl, acetyl, pivaloyl, or benzoyl.

In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group. In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each sulfur atom substituent is independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). In some embodiments, each sulfur protecting group is selected from the group consisting of —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 314 edition, John Wiley & Sons, 1999, incorporated herein by reference.

In certain embodiments, the molecular weight of a substituent is lower than 250, lower than 200, lower than 150, lower than 100, or lower than 50 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, iodine, oxygen, sulfur, and/or nitrogen atoms. In certain embodiments, a substituent consists of carbon, hydrogen, fluorine, chlorine, bromine, and/or iodine atoms. In certain embodiments, a substituent consists of carbon, hydrogen, fluorine, and/or chlorine atoms. In certain embodiments, a substituent comprises 0, 1, 2, or 3 hydrogen bond donors. In certain embodiments, a substituent comprises 0, 1, 2, or 3 hydrogen bond acceptors.

A “counterion” or “anionic counterion” is a negatively charged group associated with a positively charged group in order to maintain electronic neutrality. An anionic counterion may be monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂ or (HCB₁₁Me₅Br₆)). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

A “leaving group” (LG) is an art-understood term referring to an atomic or molecular fragment that departs with a pair of electrons in heterolytic bond cleavage, wherein the molecular fragment is an anion or neutral molecule. As used herein, a leaving group can be an atom or a group capable of being displaced by a nucleophile. See e.g., Smith, March Advanced Organic Chemistry 6th ed. (501-502). Exemplary leaving groups include, but are not limited to, halo (e.g., fluoro, chloro, bromo, iodo) and activated substituted hydroxyl groups (e.g., —OC(═O)SR^{aa}, —OC(═O))R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein). Additional examples of suitable leaving groups include, but are not limited to, halogen alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In some embodiments, the leaving group is a sulfonic acid ester, such as toluenesulfonate (tosylate, —OTs), methanesulfonate (mesylate, —OMs), p-bromobenzenesulfonyloxy (brosylate, —OBs), —OS(═O)₂(CF₂)₃CF₃ (nonaflate, —ONf), or trifluoromethanesulfonate (triflate, —OTf). In some embodiments, the leaving group is a brosylate, such as p-bromobenzenesulfonyloxy. In some embodiments, the leaving group is a nosylate, such as 2-nitrobenzenesulfonyloxy. In some embodiments, the leaving group is a sulfonate-containing group. In some embodiments, the leaving group is a tosylate group. In some embodiments, the leaving group is a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

These and other exemplary substituents are described in more detail in the Detailed Description, Examples, and Claims. The invention is not limited in any manner by the above exemplary listing of substituents.

As used herein, the term “salt” refers to any and all salts and encompasses pharmaceutically acceptable salts. The term “salt” refers to ionic compounds that result from the neutralization reaction of an acid and a base. A salt is composed of one or more cations (positively charged ions) and one or more anions (negative ions) so that the salt is electrically neutral (without a net charge). Salts of the compounds of this disclosure include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁺ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5H₂O)), and polyhydrates (x is a number greater than 1, e.g., dihydrates (R·2H₂O) and hexahydrates (R·6H₂O)).

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “co-crystal” refers to a crystalline structure comprising at least two different components (e.g., a compound and an acid), wherein each of the components is independently an atom, ion, or molecule. In certain embodiments, none of the components is a solvent. In certain embodiments, at least one of the components is a solvent. A co-crystal of a compound and an acid is different from a salt formed from a compound and the acid. In the salt, a compound is complexed with the acid in a way that proton transfer (e.g., a complete proton transfer) from the acid to a compound easily occurs at room temperature. In the co-crystal, however, a compound is complexed with the acid in a way that proton transfer from the acid to a herein does not easily occur at room temperature. In certain embodiments, in the co-crystal, there is substantially no proton transfer from the acid to a compound. In certain embodiments, in the co-crystal, there is partial proton transfer from the acid to a compound. Co-crystals may be useful to improve the properties (e.g., solubility, stability, and ease of formulation) of a compound.

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

Compounds that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers,” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.”

The term “isotopically labeled compound” refers to a derivative of a compound that only structurally differs from the compound in that at least one atom of the derivative includes at least one isotope enriched above (e.g., enriched 3-, 10-, 30-, 100-, 300-, 1,000-, 3,000- or 10,000-fold above) its natural abundance, whereas each atom of the compound includes isotopes at their natural abundances. In certain embodiments, the isotope enriched above its natural abundance is ²H. In certain embodiments, the isotope enriched above its natural abundance is ¹³C, ¹⁵N, or ¹⁸O.

The term “prodrugs” refers to compounds that have cleavable groups and become by solvolysis or under physiological conditions the compounds described herein, which are pharmaceutically active in vivo. Such examples include choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgaard, H., Design of Prodrugs, pp. 7-9, 21-24, Elsevier, Amsterdam 1985). Prodrugs include, for example, esters prepared by reaction of the parent acid with a suitable alcohol, or amides prepared by reaction of the parent acid compound with a substituted or unsubstituted amine, or acid anhydrides, or mixed anhydrides.

The terms “pharmaceutical composition,” “composition,” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “biological sample” refers to any sample including tissue samples (such as tissue sections and needle biopsies of a tissue), cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “administer,” “administering,” or “administration” refers to implanting, absorbing, ingesting, injecting, inhaling, or otherwise introducing a compound described herein, or a pharmaceutical composition thereof, in or on a subject.

The terms “treatment.” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population. In some embodiments, the subject is at risk of developing a disease or condition due to environmental factors (e.g., exposure to the sun).

An “effective amount” of a compound described herein refers to an amount sufficient to elicit the desired biological response. An effective amount of a compound described herein may vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human comprises about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for treating a disease or disorder associated with QSOX/mRNA (e.g., breast cancer (e.g., triple negative breast cancer)) in a subject in need thereof. In certain embodiments, a therapeutically effective amount is an amount effective for treating a disease or disorder associated with QSOX1 mRNA (e.g., breast cancer (e.g., triple negative breast cancer)) in a subject in need thereof. In certain embodiments, a therapeutically effective amount is an amount effective for binding RNase L in a subject in need thereof or in a cell, tissue, or biological sample (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%). In certain embodiments, a therapeutically effective amount is an amount effective for modulating QSOX1 mRNA in a subject in need thereof or in a cell, tissue, or biological sample (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%). In certain embodiments, the OSOX1 mRNA is QSOX1-a mRNA. In certain embodiments, a therapeutically effective amount is an amount effective for degrading a QSOX1 mRNA isoform in a subject in need thereof or in a cell, tissue, or biological sample (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%) In certain embodiments, the QSOX1 mRNA isoform is QSOX1-a. In certain embodiments, a therapeutically effective amount is an amount effective for inhibiting cell proliferation or promoting apoptosis in a subject in need thereof or in a cell, tissue, or biological sample (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%).

A “prophylactically effective amount” of a compound is an amount sufficient to prevent a condition, or one or more signs and/or symptoms associated with the condition or prevent its recurrence. In certain embodiments, the prophylactically effective amount is an amount that improves overall prophylaxis and/or enhances the prophylactic efficacy of another prophylactic agent. In certain embodiments, a prophylactically effective amount is an amount effective for preventing a disease or disorder associated with QSOX1 mRNA (e.g., breast cancer (e.g., triple negative breast cancer)) in a subject in need thereof. In certain embodiments, a prophylactically effective amount is an amount effective for reducing the risk of developing a disease or disorder associated with QSOX1 mRNA (e.g., breast cancer (e.g., triple negative breast cancer)) in a subject in need thereof. In certain embodiments, a prophylactically effective amount is an amount effective for binding RNase L in a subject in need thereof or in a cell, tissue, or biological sample (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%). In certain embodiments, a prophylactically effective amount is an amount effective for modulating QSOX1 mRNA in a subject in need thereof or in a cell, tissue, or biological sample (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%). In certain embodiments, the QSOX1 mRNA is QSOX1-a mRNA. In certain embodiments, a prophylactically effective amount is an amount effective for degrading a QSOX1 mRNA isoform in a subject in need thereof or in a cell, tissue, or biological sample (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%). In certain embodiments, the QSOX1 mRNA isoform is QSOX1-a. In certain embodiments, a prophylactically effective amount is an amount effective for inhibiting cell proliferation or promoting apoptosis in a subject in need thereof or in a cell, tissue, or biological sample (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%).

The term “cancer” refers to a class of diseases characterized by the development of abnormal cells that proliferate uncontrollably and have the ability to infiltrate and destroy normal body tissues. See e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma, colorectal cancer (e.g., colon cancer, rectal cancer, colorectal adenocarcinoma); connective tissue cancer; epithelial carcinoma, ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva). In some embodiments, the cancer is breast cancer (e.g., triple negative breast cancer). The compounds disclosed herein may also be useful in treating inflammation associated with cancer.

The term “gene” refers to a nucleic acid fragment that expresses a protein, including regulatory sequences preceding (5′ non-coding sequences) and following (3′ non-coding sequences) the coding sequence. “Native gene” refers to a gene as found in nature with its own regulatory sequences. “Chimeric gene” or “chimeric construct” refers to any gene or a construct, not a native gene, comprising regulatory and coding sequences that are not found together in nature. Accordingly, a chimeric gene or chimeric construct may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but which is introduced into the host organism by gene transfer. Foreign genes can comprise native genes inserted into a non-native organism, or chimeric genes. A “transgene” is a gene that has been introduced into the genome by a transformation procedure.

The terms “polynucleotide”, “nucleotide sequence”, “nucleic acid”, “nucleic acid molecule”, “nucleic acid sequence”, and “oligonucleotide” refer to a series of nucleotide bases (also called “nucleotides”) in DNA and RNA, and mean any chain of two or more nucleotides. The polynucleotides can be chimeric mixtures or derivatives or modified versions thereof, single-stranded or double-stranded. The oligonucleotide can be modified at the base moiety, sugar moiety, or phosphate backbone, for example, to improve stability of the molecule, its hybridization parameters, etc. The antisense oligonuculeotide may comprise a modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N₆-isopentenyladenine, 1-methylguanine, 1-methylinosine, 2,2-dimethylguanine, 2-methyladenine, 2-methylguanine, 3-methylcytosine, 5-methylcytosine, N₆-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N₆-isopentenyladenine, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl) uracil, a thio-guanine, and 2,6-diaminopurine. A nucleotide sequence typically carries genetic information, including the information used by cellular machinery to make proteins and enzymes. These terms include double- or single-stranded genomic and cDNA, RNA, any synthetic and genetically manipulated polynucleotide, and both sense and antisense polynucleotides. This includes single- and double-stranded molecules, i.e., DNA-DNA, DNA-RNA and RNA-RNA hybrids, as well as “protein nucleic acids” (PNAs) formed by conjugating bases to an amino acid backbone. This also includes nucleic acids containing carbohydrate or lipids. Exemplary DNAs include single-stranded DNA (ssDNA), double-stranded DNA (dsDNA), plasmid DNA (pDNA), genomic DNA (gDNA), complementary DNA (cDNA), antisense DNA, chloroplast DNA (ctDNA or cpDNA), microsatellite DNA, mitochondrial DNA (mtDNA or mDNA), kinetoplast DNA (kDNA), provirus, lysogen, repetitive DNA, satellite DNA, and viral DNA. Exemplary RNAs include single-stranded RNA (ssRNA), double-stranded RNA (dsRNA), small interfering RNA (siRNA), messenger RNA (mRNA), precursor messenger RNA (pre-mRNA), small hairpin RNA or short hairpin RNA (shRNA), microRNA (miRNA), guide RNA (gRNA), transfer RNA (tRNA), antisense RNA (asRNA), heterogeneous nuclear RNA (hnRNA), coding RNA, non-coding RNA (ncRNA), long non-coding RNA (long ncRNA or lncRNA), satellite RNA, viral satellite RNA, signal recognition particle RNA, small cytoplasmic RNA, small nuclear RNA (snRNA), ribosomal RNA (rRNA), Piwi-interacting RNA (piRNA), polyinosinic acid, ribozyme, flexizyme, small nucleolar RNA (snoRNA), spliced leader RNA, viral RNA, and viral satellite RNA.

Polynucleotides described herein may be synthesized by standard methods known in the art, e.g., by use of an automated DNA synthesizer (such as those that are commercially available from Biosearch, Applied Biosystems, etc.). As examples, phosphorothioate oligonucleotides may be synthesized by the method of Stein et al., Nucl. Acids Res., 16, 3209, (1988), methylphosphonate oligonucleotides can be prepared by use of controlled pore glass polymer supports (Sarin et al., Proc. Natl. Acad. Sci. U.S.A. 85, 7448-7451, (1988)). A number of methods have been developed for delivering antisense DNA or RNA to cells, e.g., antisense molecules can be injected directly into the tissue site, or modified antisense molecules, designed to target the desired cells (antisense linked to peptides or antibodies that specifically bind receptors or antigens expressed on the target cell surface) can be administered systemically. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines. However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong promoter. The use of such a construct to transfect target cells in the patient will result in the transcription of sufficient amounts of single stranded RNAs that will form complementary base pairs with the endogenous target gene transcripts and thereby prevent translation of the target gene mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human, cells. Such promoters can be inducible or constitutive. Any type of plasmid, cosmid, yeast artificial chromosome, or viral vector can be used to prepare the recombinant DNA construct that can be introduced directly into the tissue site.

The polynucleotides may be flanked by natural regulatory (expression control) sequences or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The nucleic acids may also be modified by many means known in the art. Non-limiting examples of such modifications include methylation, “caps”, substitution of one or more of the naturally occurring nucleotides with an analog, and internucleotide modifications, such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, isotopes (e.g., radioactive isotopes), biotin, and the like.

“RNA transcript” refers to the product resulting from RNA polymerase-catalyzed transcription of a DNA sequence. When the RNA transcript is a complementary copy of the DNA sequence, it is referred to as the primary transcript, or it may be an RNA sequence derived from post-transcriptional processing of the primary transcript and is referred to as the mature RNA. “Messenger RNA (mRNA)” refers to the RNA that is without introns and can be translated into polypeptides by the cell. “cRNA” refers to complementary RNA, transcribed from a recombinant cDNA template. “cDNA” refers to DNA that is complementary to and derived from an mRNA template. The cDNA can be single-stranded or converted to double-stranded form using, for example, the Klenow fragment of DNA polymerase I.

A sequence “complementary” to a portion of an RNA, refers to a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

The terms “nucleic acid” or “nucleic acid sequence”, “nucleic acid molecule”, “nucleic acid fragment” or “polynucleotide” may be used interchangeably with “gene”, “mRNA encoded by a gene” and “cDNA”.

The term “mRNA” or “mRNA molecule” refers to messenger RNA, or the RNA that serves as a template for protein synthesis in a cell. The sequence of a strand of mRNA is based on the sequence of a complementary strand of DNA comprising a sequence coding for the protein to be synthesized.

The term “siRNA” or “siRNA molecule” refers to small inhibitory RNA duplexes that induce the RNA interference (RNAi) pathway, where the siRNA interferes with the expression of specific genes with a complementary nucleotide sequence. siRNA molecules can vary in length (e.g., between 18-30 or 20-25 basepairs, inclusive) and contain varying degrees of complementarity to their target mRNA in the antisense strand. Some siRNA have unpaired overhanging bases on the S′ or 3′ end of the sense strand and/or the antisense strand. The term siRNA includes duplexes of two separate strands, as well as single strands that can form hairpin structures comprising a duplex region.

The term “microRNAs” or “miRNA” refers to small non-coding RNAs that are transcribed as primary transcripts that are processed first in the nucleus by a first nuclease to liberate the precursor miRNA, and then in the cytoplasm by a second nuclease to produce the mature miRNA. In certain embodiments, the term “microRNAs” or “miRNA” refers to small non-coding RNAs that are transcribed as primary transcripts that are processed first in the nucleus by Drosha to liberate the precursor miRNA, and then in the cytoplasm by Dicer to produce the mature miRNA.

The term “linker” refers to a bond or a divalent chemical moiety that is bonded to (i.e., that connects) two separate monovalent chemical moieties (e.g., B and R in Formula (II)).

The term “RNA binder” refers to a compound or chemical moiety that is capable of binding to RNA (e.g., an RNA target). In certain embodiments, binding interactions between the RNA binder and RNA are based on structure. In certain embodiments, binding interactions between the RNA binder and RNA are based on structure and not the RNA sequence. In certain embodiments, the RNA binder and RNA form a ternary complex. In certain embodiments, the RNA binder is identified using a DNA-encoded library (DEL). In certain embodiments, the RNA binder is identified using Inforna or Inforna 2.0, as described in S. P. Velagapudi et al., Nat. Chem. Biol. 2014, 10 (4): 291-97 and M. D. Disney et al., ACS Chem. Biol. 2016, 11 (6): 1720-28, the contents of which are incorporated herein by reference. In certain embodiments, the RNA binder is identified using two-dimensional combinatorial screening (2DCS), as described in M. D. Disney et al., J. Am. Chem. Soc. 2008, 130 (33): 11185-94 and International Patent Application No. PCT/US2018/000020, the contents of which are incorporated herein by reference. In certain embodiments, the RNA binder is identified using a DNA-encoded library (DEL), as described in R. I. Benhamou, et al., Proc. Natl. Acad. Sci. U.S.A. 2022, 119 (6) e2114971119, the contents of which are incorporated herein by reference. In certain embodiments, the RNA binder is identified using chemical cross-linking and isolation by pull-down (Chem-CLIP), as described in International Patent Application No. PCT/US2020/070189 and B. M. Suresh, et al., Proc. Natl. Acad. Sci. U.S.A. 2020, 117 (52): 33197-203, the contents of which are incorporated herein by reference.

### DETAILED DESCRIPTION OF CERTAIN EMBODIMENTS

The aspects described herein are not limited to specific embodiments, systems, compositions, methods, or configurations, and as such can, of course, vary. The terminology used herein is for the purpose of describing particular aspects only and, unless specifically defined herein, is not intended to be limiting.

### Methods of Transcriptome-Wide Mapping of RNA Binding Sites in a Cell and Making Modified Ribonucleic Acids

In one aspect, the present disclosure provides methods of transcriptome-wide mapping of RNA binding sites in a cell, comprising (a) providing purified total RNA from a cell; (b) combining the purified total RNA with a compound comprising a diazirine moiety and an alkyne moiety to form a mixture; (c) irradiating the mixture; (d) treating the irradiated mixture with a fluorescent dye comprising an azide moiety or an agarose comprising an azide moiety under conditions wherein triazolyl-bound RNA is formed; and (e) evaluating the resulting triazolyl-bound RNA to identify a target RNA. In certain embodiments, the method comprises, sequentially: (a) providing purified total RNA from a cell; (b) combining the purified total RNA with a compound comprising a diazirine moiety and an alkyne moiety to form a mixture; (c) irradiating the mixture; (d) treating the irradiated mixture with a fluorescent dye comprising an azide moiety or an agarose comprising an azide moiety under conditions wherein triazolyl-bound RNA is formed; and (e) evaluating the resulting triazolyl-bound RNA to identify a target RNA.

In another aspect, the present disclosure provides methods of transcriptome-wide mapping of RNA binding sites in a cell, comprising (a) providing a cell; (b) treating the cell with a compound comprising a diazirine moiety and an alkyne moiety to form a mixture; (c) irradiating the mixture; (d) treating the irradiated mixture with a fluorescent dye comprising an azide moiety or an agarose comprising an azide moiety under conditions wherein triazolyl-bound RNA is formed; and (e) evaluating the resulting triazolyl-bound RNA to identify a target RNA. In certain embodiments, the method comprises, sequentially: (a) providing a cell; (b) treating the cell with a compound comprising a diazirine moiety and an alkyne moiety to form a mixture; (c) irradiating the mixture; (d) treating the irradiated mixture with a fluorescent dye comprising an azide moiety or an agarose comprising an azide moiety under conditions wherein triazolyl-bound RNA is formed; and (e) evaluating the resulting triazolyl-bound RNA to identify a target RNA.

In certain embodiments, the method further comprises (f) harvesting from the mixture total RNA comprising the triazolyl-bound RNA; (g) fragmenting the total RNA, (h) performing pull-down of the triazolyl-bound RNA; and (i) identifying a binding site within the target RNA.

In certain embodiments, the method comprises: (a) providing purified total RNA from a cell; (b) combining the purified total RNA with a compound comprising a diazirine moiety and an alkyne moiety to form a mixture; (c) irradiating the mixture; (d) treating the irradiated mixture with a fluorescent dye comprising an azide moiety or an agarose comprising an azide moiety under conditions wherein triazolyl-bound RNA is formed; (e) harvesting from the mixture total RNA comprising the triazolyl-bound RNA; (f) fragmenting the total RNA; (g) performing pull-down of the triazolyl-bound RNA; and (h) evaluating the resulting triazolyl-bound RNA to identify a target RNA and identify a binding site within the target RNA. In certain embodiments, the method comprises, sequentially: (a) providing purified total RNA from a cell; (b) combining the purified total RNA with a compound comprising a diazirine moiety and an alkyne moiety to form a mixture; (c) irradiating the mixture; (d) treating the irradiated mixture with a fluorescent dye comprising an azide moiety or an agarose comprising an azide moiety under conditions wherein triazolyl-bound RNA is formed; (e) harvesting from the mixture total RNA comprising the triazolyl-bound RNA; (f) fragmenting the total RNA; (g) performing pull-down of the triazolyl-bound RNA; and (h) evaluating the resulting triazolyl-bound RNA to identify a target RNA and identify a binding site within the target RNA.

In certain embodiments, the method comprises: (a) providing a cell; (b) treating the cell with a compound comprising a diazirine moiety and an alkyne moiety to form a mixture; (c) irradiating the mixture; (d) treating the irradiated mixture with a fluorescent dye comprising an azide moiety or an agarose comprising an azide moiety under conditions wherein triazolyl-bound RNA is formed; (e) harvesting from the mixture total RNA comprising the triazolyl-bound RNA; (f) fragmenting the total RNA, (g) performing pull-down of the triazolyl-bound RNA; and (h) evaluating the resulting triazolyl-bound RNA to identify a target RNA and identify a binding site within the target RNA. In certain embodiments, the method comprises, sequentially: (a) providing a cell; (b) treating the cell with a compound comprising a diazirine moiety and an alkyne moiety to form a mixture; (c) irradiating the mixture; (d) treating the irradiated mixture with a fluorescent dye comprising an azide moiety or an agarose comprising an azide moiety under conditions wherein triazolyl-bound RNA is formed; (e) harvesting from the mixture total RNA comprising the triazolyl-bound RNA; (f) fragmenting the total RNA; (g) performing pull-down of the triazolyl-bound RNA; and (h) evaluating the resulting triazolyl-bound RNA to identify a target RNA and identify a binding site within the target RNA.

In certain embodiments, the cell is a cancer cell. In certain embodiments, the cell is a breast cancer cell. In certain embodiments, the cell is an MDA-MB-231 triple negative breast cancer cell.

In certain embodiments, the compound is a compound of Formula (I):

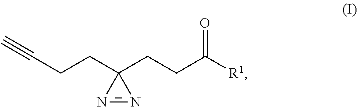

or a pharmaceutically acceptable salt thereof, wherein R¹ is selected from the group consisting of:

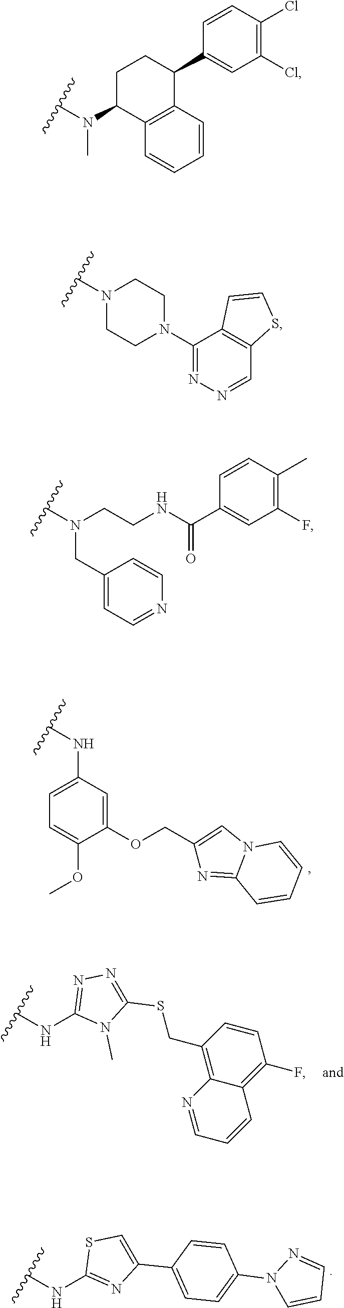 [SMILES: *N(C)[C@H]1CC[C@@H](c2ccc(Cl)c(Cl)c2)c2ccccc21 ; *N1CCN(c2nncc3sccc23)CC1 ; *N(CCNC(=O)c1ccc(C)c(F)c1)Cc1ccncc1 ; *Nc1ccc(OC)c(OCc2cn3ccccc3n2)c1 ; *Nc1nnc(SCc2ccc(F)c3cccnc23)n1C ; *Nc1nc(-c2ccc(-n3cccn3)cc2)cs1]

In certain aspects, the compound is of formula

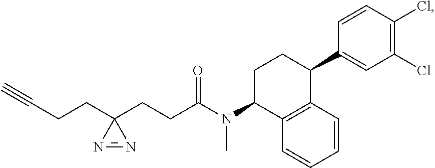 [SMILES: C#CCCC1(CCC(=O)N(C)[C@H]2CC[C@@H](c3ccc(Cl)c(Cl)c3)c3ccccc32)N=N1]

or a pharmaceutically acceptable salt thereof.

In certain embodiments, the mixture is incubated before irradiation. In certain embodiments, the incubation is for at least 10 minutes, at least 20 minutes, at least 30, at least 1 hour, at least 2 hours, at least 4 hours, at least 8 hours, at least 12 hours, at least 16 hours, at least 20 hours, or at least 24 hours. In certain embodiments, the incubation is for at least 30 minutes. In certain embodiments, the incubation is for at least 16 hours.

In certain embodiments, the irradiation is for at least 1 minute, at least 2 minutes, at least 3 minutes, at least 4 minutes, at least 5 minutes, at least 10 minutes, at least 15 minutes, at least 20 minutes, at least 30 minutes, at least 45 minutes, or at least 60 minutes. In certain embodiments, the irradiation is for at least 10 minutes. In certain embodiments, the irradiation is with ultraviolet light. In certain embodiments, treating the irradiated mixture under conditions wherein triazolyl-bound RNA is formed comprises a copper (II) salt and a reducing agent. In certain embodiments, treating the irradiated mixture under conditions wherein triazolyl-bound RNA is formed is for at least 1 hour, at least 2 hours, at least 3 hours, at least 4 hours, at least 5 hours, at least 6 hours, at least 12 hours, at least 16 hours, or at least 24 hours. In certain embodiments, treating the irradiated mixture under conditions wherein triazolyl-bound RNA is formed is for at least 3 hours.

In certain embodiments, evaluating the resulting triazolyl-bound RNA is by gel electrophoresis or fluorescence imaging. In certain embodiments, evaluating the resulting triazolyl-bound RNA is by gel electrophoresis. In certain embodiments, evaluating the resulting triazolyl-bound RNA is by fluorescence imaging. In certain embodiments, evaluating the resulting triazolyl-bound RNA comprises imaging the fluorescence of the fluorescent dye. In certain embodiments, the fluorescent dye is a cyanine, fluorescein, rhodamine, or BODIPY. In certain embodiments, the fluorescent dye is a rhodamine. In certain embodiments, the fluorescent dye is tetramethylrhodamine (TAMRA). In certain embodiments, evaluating the resulting triazolyl-bound RNA comprises imaging tetramethylrhodamine (TAMRA) fluorescence. In certain embodiments, evaluating the resulting triazolyl-bound RNA is by gel electrophoresis. In certain embodiments, the method further comprises treating the gel with an agent capable of staining the gel. In certain embodiments, the gel is stained with SYBR Green and/or Coomassie staining.

In certain embodiments, evaluating the resulting triazolyl-bound RNA comprises detecting a target signal at least 2-fold, at least 3-fold, at least 4-fold, or at least 5-fold above a background signal. In certain embodiments, evaluating the resulting triazolyl-bound RNA comprises detecting a target signal at least 3-fold above a background signal. In certain embodiments, evaluating the resulting triazolyl-bound RNA comprises identifying enrichment of the target RNA. In certain embodiments, the method is selective for enrichment of the target RNA compared to DNA or proteins.

In certain embodiments, the compound comprises a moiety capable of binding RNA. In certain embodiments, evaluating the resulting triazolyl-bound RNA comprises identifying a control target that non-specifically reacts with the diazirine moiety. In certain embodiments, the method further comprises using a control compound comprising a diazirine moiety and an alkyne moiety, wherein the control compound does not comprise a moiety capable of binding RNA, to identify a control target that non-specifically reacts with the diazirine moiety.

In certain embodiments, evaluating the resulting triazolyl-bound RNA comprises evaluating the presence or absence of triazolyl-bound RNA. In certain embodiments, evaluating the resulting triazolyl-bound RNA comprises identifying the resulting triazolyl-bound RNA. In certain embodiments, evaluating the resulting triazolyl-bound RNA further comprises isolating a triazolyl-bound RNA complex. In certain embodiments, evaluating the resulting triazolyl-bound RNA further comprises de-complexing the triazolyl-bound RNA complex to provide an RNA capable of being sequenced. In certain embodiments, evaluating the resulting triazolyl-bound RNA further comprises sequencing the RNA capable of being sequenced.

In certain embodiments, the RNA is OSOX1 mRNA. In certain embodiments, the RNA is QSOX1-a mRNA or QSOX1-b mRNA. In certain embodiments, the RNA is QSOX1-a mRNA.

In certain embodiments, fragmenting the total RNA comprises random fragmentation. In certain embodiments, performing pull-down of triazolyl-bound RNA comprises selectively pulling-down fragmented RNA regions bound by the compound.

In certain embodiments, the concentration of the compound is sufficient for the pull-down of triazolyl-bound RNA to enrich the target RNA. In certain embodiments, the concentration of the compound is at least 1 μM, at least 2 μM, at least 3 μM, at least 4 μM, at least 5 μM, at least 6 μM, at least 7 μM, at least 8 μM, at least 9 μM, at least 10 μM, at least 11 μM, at least 12 μM, at least 13 μM, at least 14 μM, at least 15 μM, at least 16 μM, at least 17 μM, at least 18 μM, at least 19 μM, at least 20 μM, at least 25 μM, at least 30 μM, at least 40 μM, or at least 50 μM. In certain embodiments, the concentration of the compound is at least 5 μM (˜6-fold). In certain embodiments, the concentration of the compound is at least 20 M (˜12-fold). In certain embodiments, the concentration of the compound is at least 5 μM (˜6-fold) or 20 μM (˜12-fold).

In certain embodiments, the pull-down of triazolyl-bound RNA is performed for a time sufficient to enrich the target RNA. In certain embodiments, the pull-down of triazolyl-bound RNA is performed for at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 36 hours, or at least 48 hours. In certain embodiments, the pull-down of triazolyl-bound RNA is performed for at least 8 hours. In certain embodiments, the pull-down of triazolyl-bound RNA is performed for at least 16 hours.

In certain embodiments, the method does not pull-down a protein capable of forming an mRNA-protein complex. In certain embodiments, the method does not pull-down a protein produced from the target RNA.

In another aspect, the present disclosure provides methods of making a modified ribonucleic acid, or a pharmaceutically acceptable salt thereof, comprising reacting a ribonucleic acid with a compound of Formula (I):

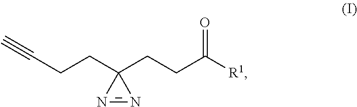

or a pharmaceutically acceptable salt thereof, wherein R¹ is selected from the group consisting of:

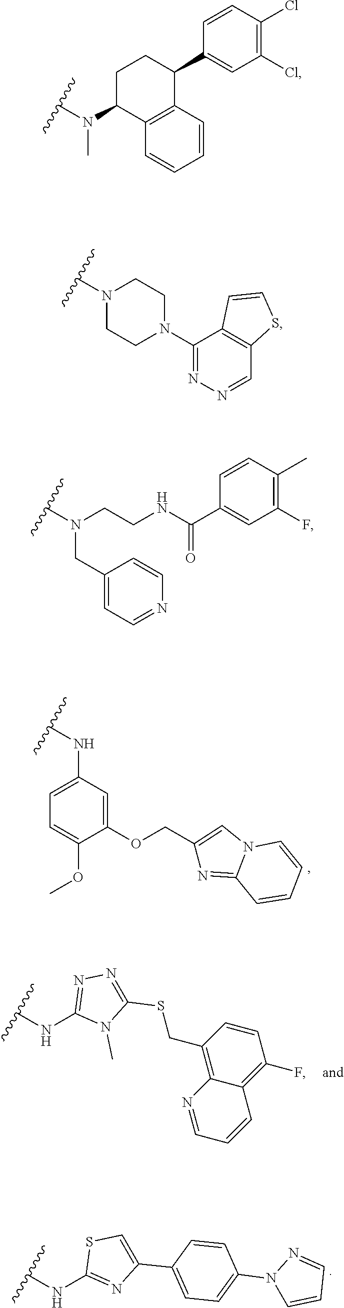 [SMILES: *N(C)[C@H]1CC[C@@H](c2ccc(Cl)c(Cl)c2)c2ccccc21 ; *N1CCN(c2nncc3sccc23)CC1 ; *N(CCNC(=O)c1ccc(C)c(F)c1)Cc1ccncc1 ; *Nc1ccc(OC)c(OCc2cn3ccccc3n2)c1 ; *Nc1nnc(SCc2ccc(F)c3cccnc23)n1C ; *Nc1nc(-c2ccc(-n3cccn3)cc2)cs1]

In another aspect, the present disclosure provides a modified ribonucleic acid, or a pharmaceutically acceptable salt thereof, made by reacting a ribonucleic acid with a compound of Formula (I):

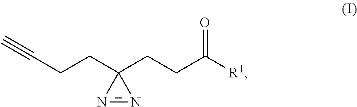

or a pharmaceutically acceptable salt thereof, wherein R¹ is selected from the group consisting of:

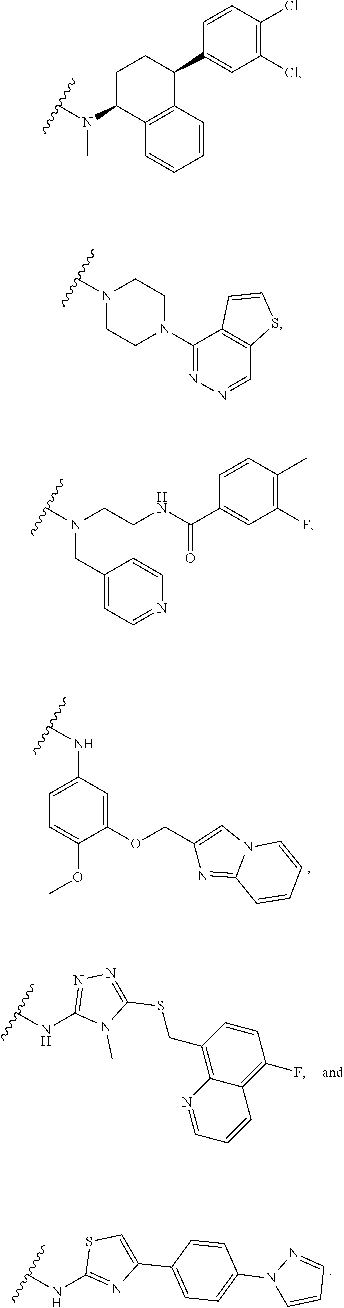 [SMILES: *N(C)[C@H]1CC[C@@H](c2ccc(Cl)c(Cl)c2)c2ccccc21 ; *N1CCN(c2nncc3sccc23)CC1 ; *N(CCNC(=O)c1ccc(C)c(F)c1)Cc1ccncc1 ; *Nc1ccc(OC)c(OCc2cn3ccccc3n2)c1 ; *Nc1nnc(SCc2ccc(F)c3cccnc23)n1C ; *Nc1nc(-c2ccc(-n3cccn3)cc2)cs1]

In another aspect, the present disclosure provides a method of making a fluorescent-tagged ribonucleic acid, comprising reacting a modified ribonucleic acid with a fluorescent dye comprising an azide moiety.

In another aspect, the present disclosure provides a method of making a modified agarose, comprising reacting a modified ribonucleic acid with an agarose comprising an azide moiety.

### Compounds

In one aspect, the present disclosure provides a compound of Formula (I):

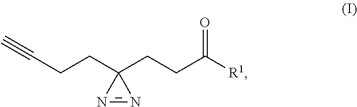

or a pharmaceutically acceptable salt thereof, wherein R¹ is selected from the group consisting of:

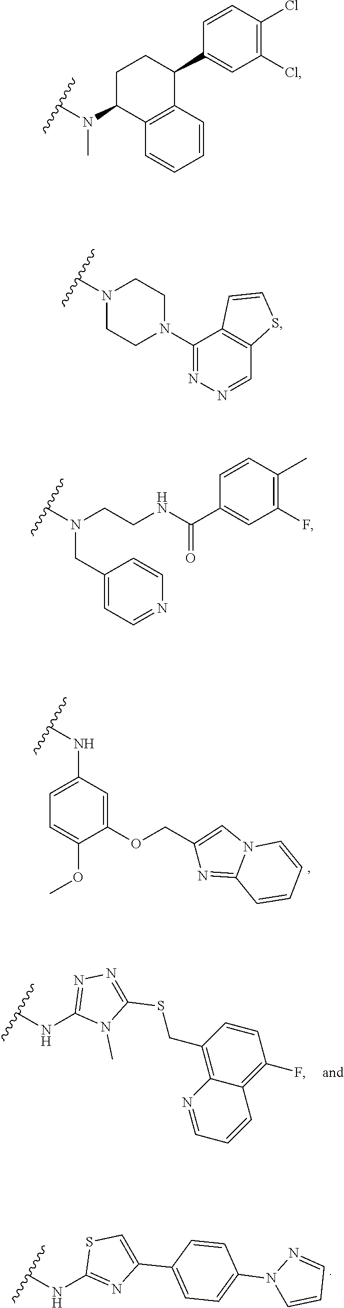 [SMILES: *N(C)[C@H]1CC[C@@H](c2ccc(Cl)c(Cl)c2)c2ccccc21 ; *N1CCN(c2nncc3sccc23)CC1 ; *N(CCNC(=O)c1ccc(C)c(F)c1)Cc1ccncc1 ; *Nc1ccc(OC)c(OCc2cn3ccccc3n2)c1 ; *Nc1nnc(SCc2ccc(F)c3cccnc23)n1C ; *Nc1nc(-c2ccc(-n3cccn3)cc2)cs1]

In certain aspects, the compound of Formula (I) is of formula:

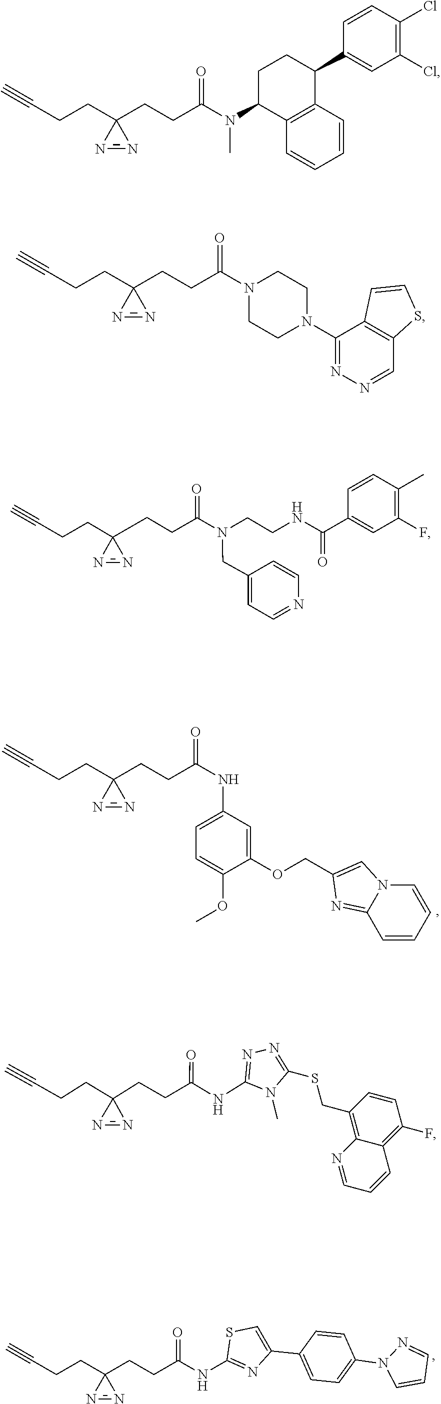 [SMILES: C#CCCC1(CCC(=O)N(C)[C@H]2CC[C@@H](c3ccc(Cl)c(Cl)c3)c3ccccc32)N=N1 ; C#CCCC1(CCC(=O)N2CCN(c3nncc4sccc34)CC2)N=N1 ; C#CCCC1(CCCC(=O)N(CCNC(=O)Cc2ccc(C)c(F)c2)Cc2ccncc2)N=N1 ; C#CCCC1(CCC(=O)Nc2ccc(OC)c(OCc3cn4ccccc4n3)c2)N=N1 ; C#CCCC1(CCC(=O)Nc2nnc(SCc3ccc(F)c4cccnc34)n2C)N=N1 ; C#CCCC1(CCC(=O)Nc2nc(-c3ccc(-n4cccn4)cc3)cs2)N=N1]

or a pharmaceutically acceptable salt thereof.

In certain aspects, the compound of Formula (I) is of formula

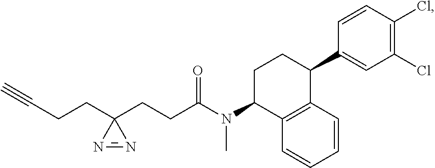 [SMILES: C#CCCC1(CCC(=O)N(C)[C@H]2CC[C@@H](c3ccc(Cl)c(Cl)c3)c3ccccc32)N=N1]

or a pharmaceutically acceptable salt thereof. In certain aspects, the compound of Formula (I) is of formula

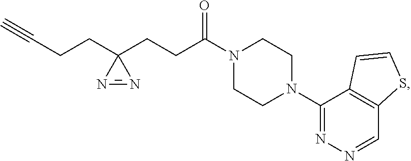 [SMILES: C#CCCC1(CCC(=O)N2CCN(c3nncc4sccc34)CC2)N=N1]

or a pharmaceutically acceptable salt thereof. In certain aspects, the compound of Formula (I) is of formula

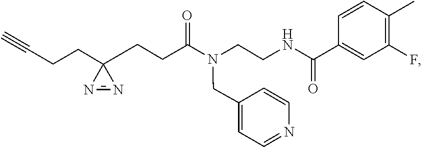 [SMILES: C#CCCC1(CCCC(=O)N(CCNC(=O)Cc2ccc(C)c(F)c2)Cc2ccncc2)N=N1]

or a pharmaceutically acceptable salt thereof. In certain aspects, the compound of Formula (I) is of formula

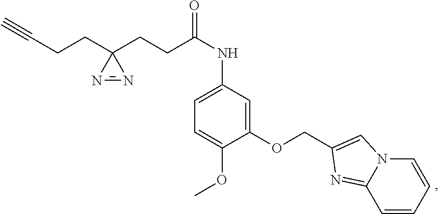 [SMILES: C#CCCC1(CCC(=O)Nc2ccc(OC)c(OCc3cn4ccccc4n3)c2)N=N1]

or a pharmaceutically acceptable salt thereof. In certain aspects, the compound of Formula (I) is of formula

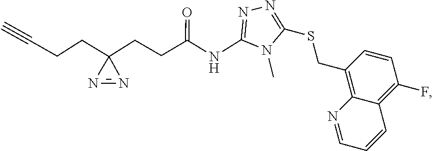 [SMILES: C#CCCC1(CCC(=O)Nc2nnc(SCc3ccc(F)c4cccnc34)n2C)N=N1]

or a pharmaceutically acceptable salt thereof. In certain aspects, the compound of Formula (I) is of formula

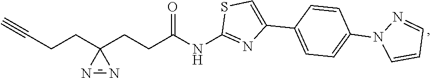 [SMILES: C#CCCC1(CCC(=O)Nc2nc(-c3ccc(-n4cccn4)cc3)cs2)N=N1]

or a pharmaceutically acceptable salt thereof.

In another aspect, the present disclosure provides a compound of Formula (II):

B-L-R (II),

or a pharmaceutically acceptable salt thereof, wherein:
B is an RNA binder of formula:

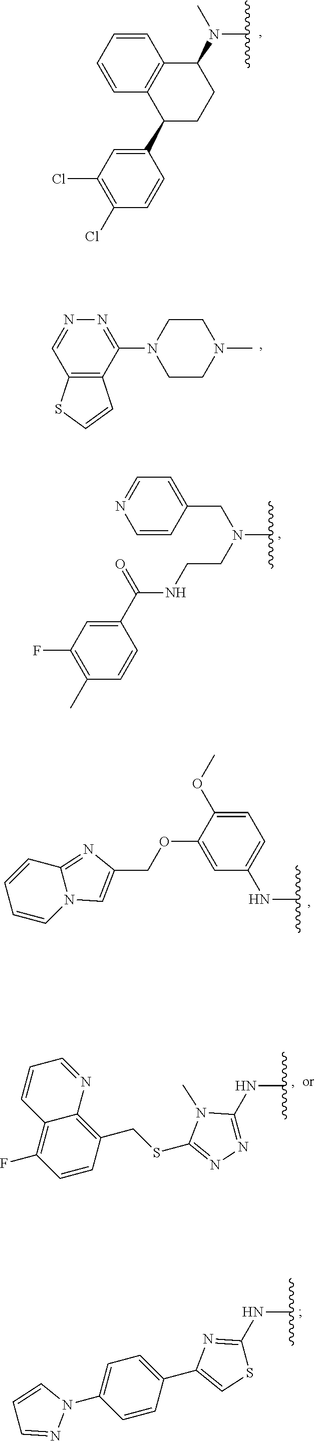 [SMILES: *N(C)[C@H]1CC[C@@H](c2ccc(Cl)c(Cl)c2)c2ccccc21 ; CN1CCN(c2nncc3sccc23)CC1 ; *N(CCNC(=O)c1ccc(C)c(F)c1)Cc1ccncc1 ; *Nc1ccc(OC)c(OCc2cn3ccccc3n2)c1 ; *Nc1nnc(SCc2ccc(F)c3cccnc23)n1C ; *Nc1nc(-c2ccc(-n3cccn3)cc2)cs1]

L is a linker, and
R is an RNase L recruiter.

In certain embodiments, B is an RNA binder of formula

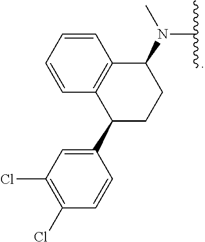

In certain embodiments, B is an RNA binder of formula

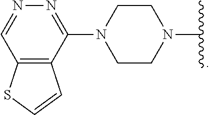 [SMILES: *N1CCN(c2nncc3sccc23)CC1]

In certain embodiments, B is an RNA binder of formula

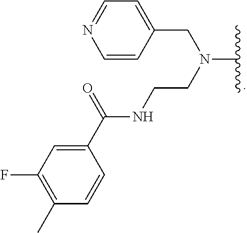 [SMILES: *N(CCNC(=O)c1ccc(C)c(F)c1)Cc1ccncc1]

In certain embodiments, B is an RNA binder of formula

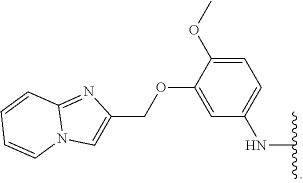 [SMILES: *Nc1ccc(OC)c(OCc2cn3ccccc3n2)c1]

In certain embodiments, B is an RNA binder of formula

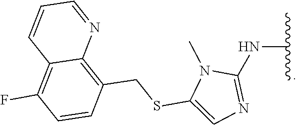 [SMILES: *Nc1ncc(SCc2ccc(F)c3cccnc23)n1C]

In certain embodiments, B is an RNA binder of formula

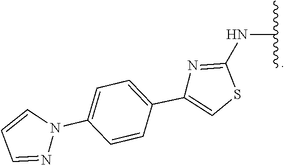 [SMILES: *Nc1nc(-c2ccc(-n3cccn3)cc2)cs1]

In certain embodiments, L is a linker. In certain embodiments, L is a bond, optionally substituted alkylene, optionally substituted alkenylene, optionally substituted alkynylene, optionally substituted heteroalkylene, optionally substituted heteroalkenylene, optionally substituted heteroalkynylene, optionally substituted heterocyclylene, optionally substituted carbocyclylene, optionally substituted arylene, optionally substituted heteroarylene, or a combination thereof. In certain embodiments, L is a bond. In certain embodiments, L is optionally substituted alkylene. In certain embodiments, L is optionally substituted alkenylene. In certain embodiments, L is optionally substituted alkynylene. In certain embodiments, L is optionally substituted heteroalkylene. In certain embodiments, L is optionally substituted heteroalkenylene. In certain embodiments, L is optionally substituted heteroalkynylene. In certain embodiments, L is optionally substituted heterocyclylene. In certain embodiments, L is optionally substituted carbocyclylene. In certain embodiments, L is optionally substituted arylene. In certain embodiments, L is optionally substituted heteroarylene. In certain embodiments, L is optionally substituted alkylene, optionally substituted heteroalkylene, or a combination thereof.

In certain embodiments, L is of Formula (III):

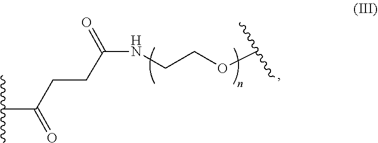

wherein n is 0, 1, 2, 3, 4, 5, 6, 7, 8, 9, or 10. In certain embodiments, n is 0. In certain embodiments, n is 1. In certain embodiments, n is 2. In certain embodiments, n is 3. In certain embodiments, n is 4. In certain embodiments, n is 5. In certain embodiments, n is 6. In certain embodiments, n is 7. In certain embodiments, n is 8. In certain embodiments, n is 9. In certain embodiments, n is 10.

In certain embodiments, L is of formula

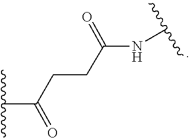 [SMILES: *NC(=O)CCC(*)=O]

In certain embodiments, L is of formula

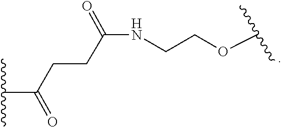 [SMILES: *OCCNC(=O)CCC(*)=O]

In certain embodiments, L is of formula

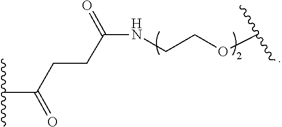

In certain embodiments, L is of formula

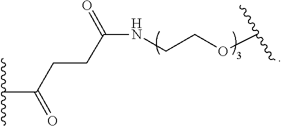

In certain embodiments, L is of formula

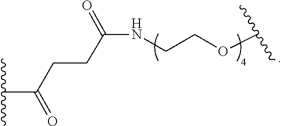

In certain embodiments, L is of formula

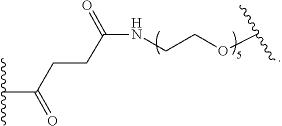

In certain embodiments, L is of formula

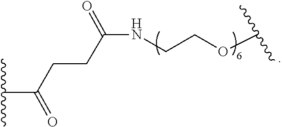

In certain embodiments, L is of formula

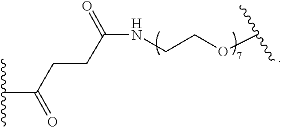

In certain embodiments, L is of formula

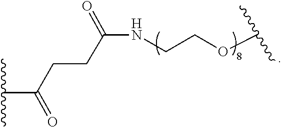

In certain embodiments, L is of formula

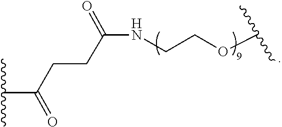

In certain embodiments, L is of formula

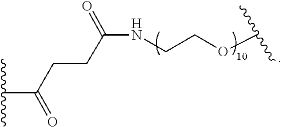

In certain embodiments. R is an RNAse L recruiter of Formulae (IV-a) or (IV-b):

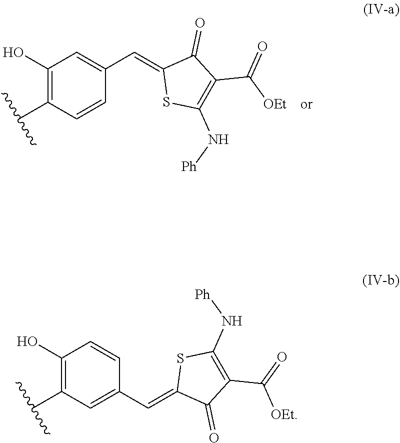 [SMILES: *c1ccc(/C=C2\SC(Nc3ccccc3)=C(C(=O)OCC)C2=O)cc1O ; *c1cc(/C=C2\SC(Nc3ccccc3)=C(C(=O)OCC)C2=O)ccc1O]

In certain embodiments, R is an RNAse L recruiter of Formula (IV-a):

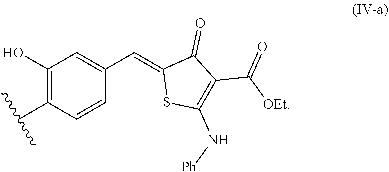 [SMILES: *c1ccc(/C=C2\SC(Nc3ccccc3)=C(C(=O)OCC)C2=O)cc1O]

In certain embodiments, R is an RNAse L recruiter of Formula (IV-b):

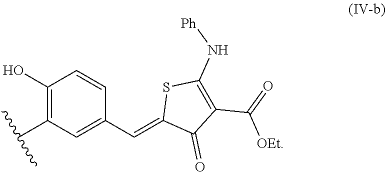 [SMILES: *c1cc(/C=C2\SC(Nc3ccccc3)=C(C(=O)OCC)C2=O)ccc1O]

In certain embodiments, the compound of Formula (II) is of Formula (II-a):

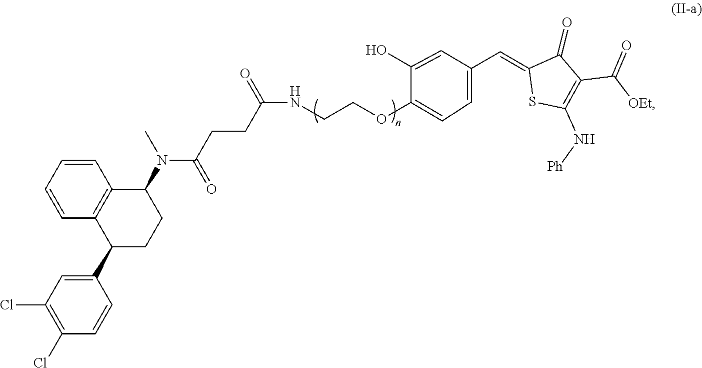

or a pharmaceutically acceptable salt thereof, wherein n is 0, 1, 2, 3, 4, 5, 6, 7, 8, 9, or 10. In certain embodiments, n is 0. In certain embodiments, n is 1. In certain embodiments, n is 2. In certain embodiments, n is 3. In certain embodiments, n is 4. In certain embodiments, n is 5. In certain embodiments, n is 6. In certain embodiments, n is 7. In certain embodiments, n is 8. In certain embodiments, n is 9. In certain embodiments, n is 10.

In certain embodiments, the compound of Formula (II) is of formula:

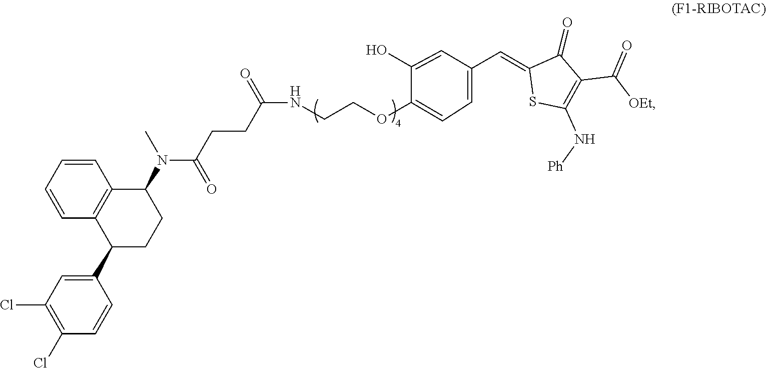

or a pharmaceutically acceptable salt prodrug thereof.

In certain embodiments, the compound of Formula (II) is of Formula (II-b):

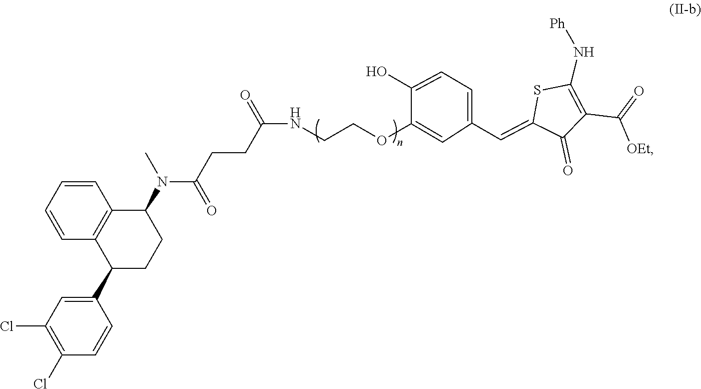

or a pharmaceutically acceptable salt thereof, wherein n is 0, 1, 2, 3, 4, 5, 6, 7, 8, 9, or 10. In certain embodiments, n is 0. In certain embodiments, n is 1. In certain embodiments, n is 2. In certain embodiments, n is 3. In certain embodiments, n is 4. In certain embodiments, n is 5. In certain embodiments, n is 6. In certain embodiments, n is 7. In certain embodiments, n is 8. In certain embodiments, n is 9. In certain embodiments, n is 10.

In certain embodiments, the compound of Formula (II) is of formula:

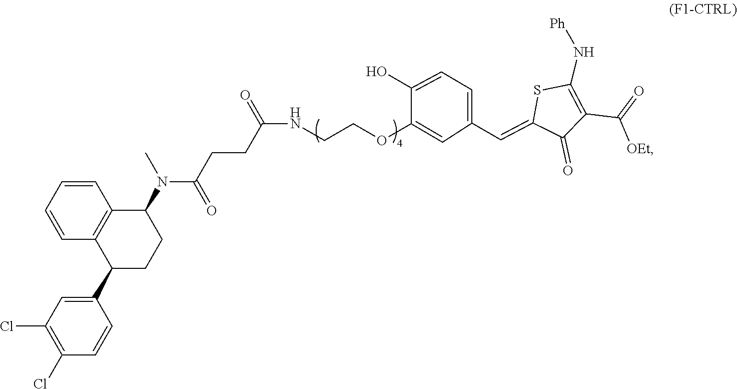

or a pharmaceutically acceptable salt thereof.

In certain embodiments, the compound of Formula (II) is of Formula (II-c):

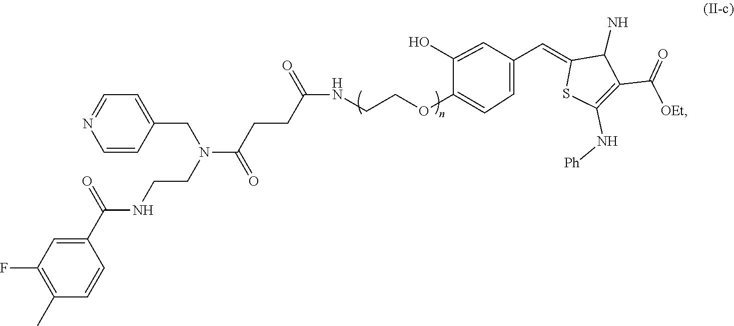

or a pharmaceutically acceptable salt thereof, wherein n is 0, 1, 2, 3, 4, 5, 6, 7, 8, 9, or 10. In certain embodiments, n is 0. In certain embodiments, n is 1. In certain embodiments, n is 2. In certain embodiments, n is 3. In certain embodiments, n is 4. In certain embodiments, n is 5. In certain embodiments, n is 6. In certain embodiments, n is 7. In certain embodiments, n is 8. In certain embodiments, n is 9. In certain embodiments, n is 10.

In certain embodiments, the compound of Formula (II) is of formula:

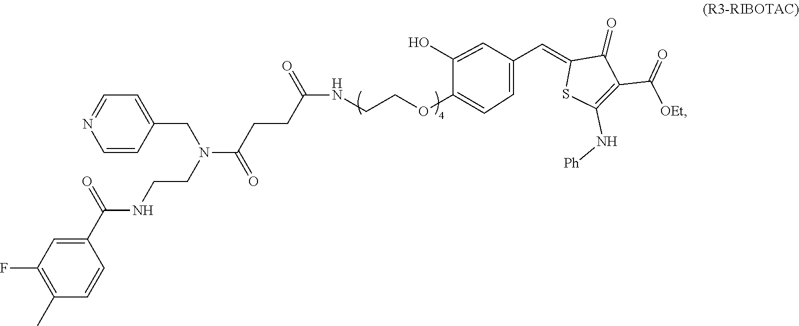

or a pharmaceutically acceptable salt thereof.

In certain embodiments, the compound of Formula (II) is of Formula (II-d):

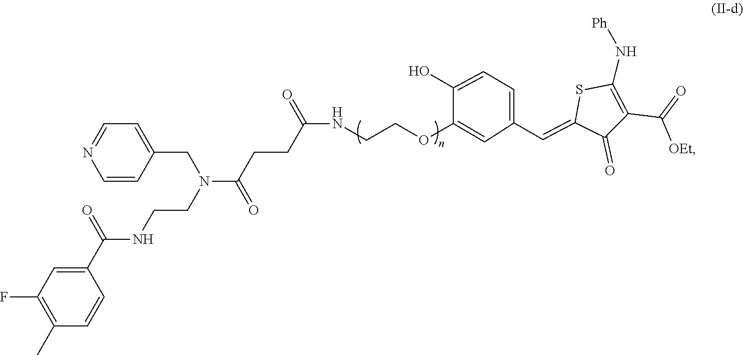

or a pharmaceutically acceptable salt thereof, wherein n is 0, 1, 2, 3, 4, 5, 6, 7, 8, 9, or 10. In certain embodiments, n is 0. In certain embodiments, n is 1. In certain embodiments, n is 2. In certain embodiments, n is 3. In certain embodiments, n is 4. In certain embodiments, n is 5. In certain embodiments, n is 6. In certain embodiments, n is 7. In certain embodiments, n is 8. In certain embodiments, n is 9. In certain embodiments, n is 10.

In certain embodiments, the compound of Formula (II) is of formula:

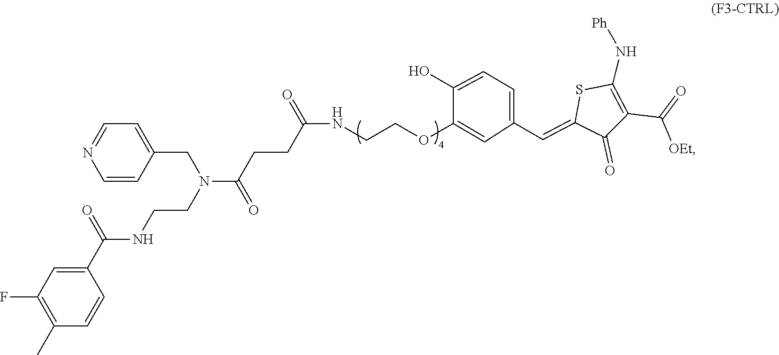

or a pharmaceutically acceptable salt thereof.

In certain embodiments, a provided compound (a compound described herein, a compound of the present disclosure) is a compound of any of the formulae herein (e.g., Formulae (I) or (II)), or pharmaceutically acceptable salt thereof. In certain embodiments, a provided compound is a compound of any of the formulae herein (e.g., Formulae (I) or (II)), or a pharmaceutically acceptable salt or tautomer thereof. In certain embodiments, a provided compound is a compound of any of the formulae herein (e.g., Formulae (I) or (II)), or a pharmaceutically acceptable salt thereof. In certain embodiments, a provided compound is a compound of any of the formulae herein (e.g., Formulae (I) or (II)), or a salt thereof.

### Pharmaceutical Compositions and Kits

In one aspect, the present disclosure provides pharmaceutical compositions comprising a provided compound. In some embodiments, the pharmaceutical composition comprises one or more excipients. In certain embodiments, the pharmaceutical compositions described herein comprise a provided compound and an excipient.

In certain embodiments, the pharmaceutical composition comprises an effective amount of the provided compound. In certain embodiments, the effective amount is a therapeutically effective amount. In certain embodiments, the effective amount is a prophylactically effective amount. In certain embodiments, the effective amount is an amount effective for binding RNase L in a subject in need thereof or in a cell, tissue, or biological sample. In certain embodiments, the effective amount is an amount effective for modulating QSOX1 mRNA in a subject in need thereof or in a cell, tissue, or biological sample. In certain embodiments, the QSOX1 mRNA is QSOX1-a mRNA. In certain embodiments, the effective amount is an amount effective for degrading a QSOX1 mRNA isoform in a subject in need thereof or in a cell, tissue, or biological sample. In certain embodiments, the QSOX1 mRNA isoform is QSOX1-a. In certain embodiments, the effective amount is an amount effective for inhibiting cell proliferation or promoting apoptosis in a subject in need thereof or in a cell, tissue, or biological sample. In certain embodiments, the effective amount is an amount effective for treating a disease or disorder associated with QSOX1 mRNA (e.g., breast cancer (e.g., triple negative breast cancer)) in a subject in need thereof. In certain embodiments, the effective amount is an amount effective for preventing a disease or disorder associated with OSOX1 mRNA (e.g., breast cancer (e.g., triple negative breast cancer)) in a subject in need thereof. In certain embodiments, the effective amount is an amount effective for reducing the risk of developing a disease or disorder associated with QSOX1 mRNA (e.g., breast cancer (e.g., triple negative breast cancer)) in a subject in need thereof.

In certain embodiments, the subject is an animal. In certain embodiments, the subject is a human. In certain embodiments, the subject is a human aged 18 years or older. In certain embodiments, the subject is a human aged 12-18 years, exclusive. In certain embodiments, the subject is a human aged 2-12 years, inclusive. In certain embodiments, the subject is a human younger than 2 years. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In certain embodiments, the effective amount is an amount effective for degrading a QSOX1 mRNA isoform in a subject in need thereof or in a cell, tissue, or biological sample (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%). In certain embodiments, the effective amount is an amount effective for degrading a QSOX1 mRNA isoform by a range between a percentage described in this paragraph and another percentage described in this paragraph, inclusive.

In certain embodiments, the pharmaceutical composition is for use in treating a disease or disorder associated with OSOX1 mRNA (e.g., breast cancer (e.g., triple negative breast cancer)) in a subject in need thereof. In certain embodiments, the pharmaceutical composition is for use in preventing a disease or disorder associated with QSOX1 mRNA (e.g., breast cancer (e.g., triple negative breast cancer)) in a subject in need thereof.

A provided compound or pharmaceutical composition, as described herein, can be administered in combination with one or more additional pharmaceutical agents (e.g., therapeutically and/or prophylactically active agents). The provided compounds or pharmaceutical compositions can be administered in combination with additional pharmaceutical agents that improve their activity (e.g., activity (e.g., potency and/or efficacy) in treating a disease or disorder associated with QSOX1 mRNA (e.g., breast cancer (e.g., triple negative breast cancer)) in a subject in need thereof, in preventing a disease or disorder associated with QSOX1 mRNA (e.g., breast cancer (e.g., triple negative breast cancer)) in a subject in need thereof, and/or in reducing the risk of developing a disease or disorder associated with QSOX1 mRNA (e.g., breast cancer (e.g., triple negative breast cancer)) in a subject in need thereof, improve bioavailability, improve safety, reduce drug resistance, reduce and/or modify metabolism, inhibit excretion, and/or modify distribution in a subject or cell. It will also be appreciated that the additional pharmaceutical agents employed may achieve a desired effect for the same disorder, and/or it may achieve different effects. In certain embodiments, a pharmaceutical composition described herein including a provided compound described herein and an additional pharmaceutical agent exhibit a synergistic effect that is absent in a pharmaceutical composition including one of the provided compounds and the additional pharmaceutical agent, but not both. In some embodiments, the additional pharmaceutical agent achieves a desired effect for the same disorder. In some embodiments, the additional pharmaceutical agent achieves different effects.

The provided compound or pharmaceutical composition can be administered concurrently with, prior to, or subsequent to one or more additional pharmaceutical agents, which are different from the compound or pharmaceutical composition and may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides, synthetic proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease or disorder associated with QSOX1 mRNA (e.g., breast cancer (e.g., triple negative breast cancer)).

Each additional pharmaceutical agent may be administered at a dose and/or on a time schedule determined for that pharmaceutical agent. The additional pharmaceutical agents may also be administered together with each other and/or with the compound or pharmaceutical composition described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, anti-angiogenesis agents, steroidal or non-steroidal anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, anesthetics, anti-coagulants, inhibitors of an enzyme, steroidal agents, steroidal or antihistamine, antigens, vaccines, antibodies, decongestant, sedatives, opioids, analgesics, anti-pyretics, hormones, and prostaglandins.

In certain embodiments, the provided compound or pharmaceutical composition is a solid. In certain embodiments, the provided compound or pharmaceutical composition is a powder. In certain embodiments, the provided compound or pharmaceutical composition can be dissolved in a liquid to make a solution. In certain embodiments, the provided compound or pharmaceutical composition is dissolved in water to make an aqueous solution. In certain embodiments, the pharmaceutical composition is a liquid for parental injection. In certain embodiments, the pharmaceutical composition is a liquid for oral administration (e.g., ingestion). In certain embodiments, the pharmaceutical composition is a liquid (e.g., aqueous solution) for intravenous injection. In certain embodiments, the pharmaceutical composition is a liquid (e.g., aqueous solution) for subcutaneous injection.

Pharmaceutical compositions described herein can be prepared by any method known in the art of pharmacology. In general, such preparatory methods include the steps of bringing the composition comprising a provided compound (i.e., the “active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the provided compound, pharmaceutically acceptable excipient, agent, and/or any additional ingredients in a pharmaceutical composition described herein will vary, depending upon the identity, size, and/or condition of the subject treated and further depending upon the route by which the pharmaceutical composition is to be administered. The pharmaceutical composition may comprise between 0.1% and 100% (w/w) agent, inclusive.

Pharmaceutically acceptable excipients used in manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients and accessory ingredients, such as cocoa butter and suppository waxes, coloring agents, coating agents, sweetening, flavoring, and perfuming agents, may also be present in the pharmaceutical composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween 20), polyoxyethylene sorbitan monostearate (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthom, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral pharmaceutical compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid pharmaceutical compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Pharmaceutical compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

Suitable devices for use in delivering injectable pharmaceutical compositions described herein include short needle devices. Injectable pharmaceutical compositions can be administered by devices which limit the effective penetration length of a needle into the skin. Alternatively or additionally, conventional syringes can be used in the classical mantoux method of administration. Jet injection devices which deliver liquid formulations via a liquid jet injector and/or via a needle. Ballistic powder/particle delivery devices which use compressed gas to accelerate the compound in powder form are suitable.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such pharmaceutical compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder pharmaceutical compositions may include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the pharmaceutical composition, and the active ingredient may constitute 0.1 to 20% (w/w) of the pharmaceutical composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such pharmaceutical compositions are generally suitable for administration to animals of all sorts. Modification of pharmaceutical compositions suitable for administration to humans in order to render the pharmaceutical compositions suitable for administration to various animals is well understood, and the ordinarily skilled veterinary pharmacologist can design and/or perform such modification with ordinary experimentation.

Provided compounds are typically formulated in dosage unit form for ease of administration and uniformity of dosage. It will be understood, however, that the total daily usage of the pharmaceutical compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The provided compounds and pharmaceutical compositions provided herein can be administered by any route, including enteral (e.g., oral), parenteral, intravenous, intramuscular, intraarticular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically, contemplated routes are intraarticular administration, oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration).

The exact amount of a provided compound required to achieve an effective amount will vary from subject to subject, depending, for example, on species, age, and general condition of a subject, severity of the side effects or disorder, identity of the particular compound of the disclosure, mode of administration, and the like. An effective amount may be included in a single dose (e.g., single oral dose) or multiple doses (e.g., multiple oral doses). In certain embodiments, when multiple doses are administered to a subject or applied to a biological sample, tissue, or cell, any two doses of the multiple doses include different or substantially the same amounts of an agent described herein.

In certain embodiments, a pharmaceutical composition comprising a provided compound is administered, orally or parenterally, at dosage levels of each pharmaceutical composition sufficient to deliver from about 0.001 mg/kg to about 200 mg/kg in one or more dose administrations for one or several days (depending on the mode of administration). In certain embodiments, the effective amount per dose varies from about 0.001 mg/kg to about 200 mg/kg, about 0.001 mg/kg to about 100 mg/kg, about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, of subject body weight per day, one or more times a day, to obtain the desired therapeutic and/or prophylactic effect. In certain embodiments, the compounds described herein may be at dosage levels sufficient to deliver from about 0.001 mg/kg to about 200 mg/kg, from about 0.001 mg/kg to about 100 mg/kg, from about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic and/or prophylactic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). In certain embodiments, the pharmaceutical composition described herein is administered at a dose that is below the dose at which the agent causes non-specific effects.

In certain embodiments, the pharmaceutical composition is administered at a dose of about 0.001 mg to about 1000 mg per unit dose. In certain embodiments, the pharmaceutical composition is administered at a dose of about 0.01 mg to about 200 mg per unit dose. In certain embodiments, the pharmaceutical composition is administered at a dose of about 0.01 mg to about 100 mg per unit dose. In certain embodiments, pharmaceutical composition is administered at a dose of about 0.01 mg to about 50 mg per unit dose. In certain embodiments, the pharmaceutical composition is administered at a dose of about 0.01 mg to about 10 mg per unit dose. In certain embodiments, the pharmaceutical composition is administered at a dose of about 0.1 mg to about 10 mg per unit dose.

Dose ranges as described herein provide guidance for the administration of provided compounds or pharmaceutical compositions to an adult. The amount to be administered to, for example, a child or an adolescent can be determined by a medical practitioner or person skilled in the art and can be lower or the same as that administered to an adult. In certain embodiments, a dose described herein is a dose to an adult human whose body weight is 70 kg.

In certain embodiments, when multiple doses are administered to a subject or applied to a tissue or cell, the frequency of administering the multiple doses to the subject or applying the multiple doses to the tissue or cell may be, in non-limiting examples, three doses a day, two doses a day, one dose a day, one dose every other day, one dose every third day, one dose every week, one dose every two weeks, one dose every three weeks, or one dose every four weeks, or even slow dose controlled delivery over a selected period of time using a drug delivery device. In certain embodiments, when multiple doses are administered to a subject or applied to a biological sample, tissue, or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell.

Also encompassed by the present disclosure are kits (e.g., pharmaceutical packs). In certain embodiments, the kit comprises a provided compound or pharmaceutical composition described herein, and instructions for using the compound or pharmaceutical composition. In certain embodiments, the kit comprises a first container, wherein the first container includes the compound or pharmaceutical composition. In some embodiments, the kit further comprises a second container. In certain embodiments, the second container includes an excipient (e.g., an excipient for dilution or suspension of the compound or pharmaceutical composition). In certain embodiments, the second container includes an additional pharmaceutical agent. In some embodiments, the kit further comprises a third container. In certain embodiments, the third container includes an additional pharmaceutical agent. In some embodiments, the provided compound or pharmaceutical composition included in the first container and the excipient or additional pharmaceutical agent included in the second container are combined to form one unit dosage form. In some embodiments, the provided compound or pharmaceutical composition included in the first container, the excipient included in the second container, and the additional pharmaceutical agent included in the third container are combined to form one unit dosage form. In certain embodiments, each of the first, second, and third containers is independently a vial, ampule, bottle, syringe, dispenser package, tube, or inhaler.

In certain embodiments, the instructions are for administering the provided compound or pharmaceutical composition to a subject (e.g., a subject in need of treatment or prevention of a disease described herein). In certain embodiments, the instructions are for contacting a biological sample or cell with the provided compound or pharmaceutical composition. In certain embodiments, the instructions comprise information required by a regulatory agency, such as the U.S. Food and Drug Administration (FDA) or the European Agency for the Evaluation of Medicinal Products (EMA). In certain embodiments, the instructions comprise prescribing information.

In certain embodiments, the kits and instructions provide for treating a disease or disorder associated with QSOX1 mRNA (e.g., breast cancer (e.g., triple negative breast cancer)) in a subject in need thereof. In certain embodiments, the kits and instructions provide for preventing a disease or disorder associated with QSOX1 mRNA (e.g., breast cancer (e.g., triple negative breast cancer)) in a subject in need thereof. In certain embodiments, the kits and instructions provide for reducing the risk of developing a disease or disorder associated with QSOX1 mRNA (e.g., breast cancer (e.g., triple negative breast cancer)) in a subject in need thereof.

A kit described herein may include one or more additional pharmaceutical agents described herein as a separate pharmaceutical composition.

Another object of the present disclosure is the use of a compound as described herein in the manufacture of a medicament for use in the treatment of a disorder or disease described herein. Another object of the present disclosure is the use of a compound as described herein for use in the treatment of a disorder or disease described herein.

Methods of Binding RNase L, Modulating QSOX1 mRNA, Degrading QSOX1 mRNA Isoforms, and Inhibiting Cell Proliferation or Promoting Apoptosis

In another aspect, the present disclosure provides methods of binding RNase L in a subject in need thereof or in a cell, tissue, or biological sample, comprising administering to the subject in need thereof or contacting the cell, tissue, or biological sample with an effective amount of a provided compound or composition. In certain embodiments, the present disclosure provides methods of binding RNase L in a subject in need thereof, comprising administering to the subject in need thereof an effective amount of a provided compound or composition. In certain embodiments, the present disclosure provides methods of binding RNase L in a cell, tissue, or biological sample, comprising contacting the cell, tissue, or biological sample with an effective amount of a provided compound or composition. In certain embodiments, the present disclosure provides a provided compound or composition for use in binding RNase L in a subject in need thereof or in a cell, tissue, or biological sample. In certain embodiments, the present disclosure provides a provided compound or composition for use in the manufacture of a medicament for binding RNase L in a subject in need thereof or in a cell, tissue, or biological sample. In certain embodiments, binding RNase L comprises activating RNase L. In certain embodiments, binding RNase L comprises inducing RNase L dimerization. In certain embodiments, the method further comprises modulating QSOX1-a mRNA. In certain embodiments, the method further comprises degrading QSOX1-a mRNA.

In another aspect, the present disclosure provides methods of modulating QSOX1 mRNA in a subject in need thereof or in a cell, tissue, or biological sample, comprising administering to the subject in need thereof or contacting the cell, tissue, or biological sample with an effective amount of a provided compound or composition. In certain embodiments, the present disclosure provides methods of modulating QSOX1 mRNA in a subject in need thereof, comprising administering to the subject in need thereof an effective amount of a provided compound or composition. In certain embodiments, the present disclosure provides methods of modulating OSOX1 mRNA in a cell, tissue, or biological sample, comprising contacting the cell, tissue, or biological sample with an effective amount of a provided compound or composition. In certain embodiments, the present disclosure provides a provided compound or composition for use in modulating QSOX1 mRNA in a subject in need thereof or in a cell, tissue, or biological sample. In certain embodiments, the present disclosure provides a provided compound or composition for use in the manufacture of a medicament for modulating QSOX1 mRNA in a subject in need thereof or in a cell, tissue, or biological sample. In certain embodiments, the QSOX1 mRNA is QSOX1-a mRNA.

In another aspect, the present disclosure provides methods of degrading a QSOX1 mRNA isoform in a subject in need thereof or in a cell, tissue, or biological sample, comprising administering to the subject in need thereof or contacting the cell, tissue, or biological sample with an effective amount of a provided compound or composition. In certain embodiments, the present disclosure provides methods of degrading a QSOX1 mRNA isoform in a subject in need thereof, comprising administering to the subject in need thereof an effective amount of a provided compound or composition. In certain embodiments, the present disclosure provides methods of degrading a QSOX1 mRNA isoform in a cell, tissue, or biological sample, comprising contacting the cell, tissue, or biological sample with an effective amount of a provided compound or composition. In certain embodiments, the present disclosure provides a provided compound or composition for use in degrading a QSOX1 mRNA isoform in a subject in need thereof or in a cell, tissue, or biological sample. In certain embodiments, the present disclosure provides a provided compound or composition for use in the manufacture of a medicament for degrading a QSOX1 mRNA isoform in a subject in need thereof or in a cell, tissue, or biological sample. In certain embodiments, the QSOX1 mRNA isoform is QSOX1-a.

In another aspect, the present disclosure provides methods of inhibiting cell proliferation or promoting apoptosis in a subject in need thereof or in a cell, tissue, or biological sample, comprising administering to the subject in need thereof or contacting the cell, tissue, or biological sample with an effective amount of a provided compound or composition. In certain embodiments, the present disclosure provides methods of inhibiting cell proliferation or promoting apoptosis in a subject in need thereof, comprising administering to the subject in need thereof an effective amount of a provided compound or composition. In certain embodiments, the present disclosure provides methods of inhibiting cell proliferation or promoting apoptosis in a cell, tissue, or biological sample, comprising contacting the cell, tissue, or biological sample with an effective amount of a provided compound or composition. In certain embodiments, the present disclosure provides a provided compound or composition for use in inhibiting cell proliferation or promoting apoptosis in a subject in need thereof or in a cell, tissue, or biological sample. In certain embodiments, the present disclosure provides a provided compound or composition for use in the manufacture of a medicament for inhibiting cell proliferation or promoting apoptosis in a subject in need thereof or in a cell, tissue, or biological sample.

In certain embodiments, the method further comprises reducing an amount of QSOX1 protein. In certain embodiments, the QSOX1 protein is QSOX1-a. In certain embodiments, the QSOX1 protein is QSOX1-b.

In certain embodiments, the cell, tissue, or biological sample is in vivo. In certain embodiments, the cell, tissue, or biological sample is in vitro.

### Methods of Treatment and Prevention

In another aspect, the present disclosure provides methods of treating or preventing a disease in a subject in need thereof, comprising administering to the subject in need thereof a provided compound or pharmaceutical composition. In certain embodiments, the present disclosure provides methods of treating a disease in a subject in need thereof, comprising administering to the subject in need thereof a provided compound or pharmaceutical composition. In certain embodiments, the present disclosure provides methods of preventing a disease in a subject in need thereof, comprising administering to the subject in need thereof a provided compound or pharmaceutical composition.

In another aspect, the present disclosure provides a provided compound or pharmaceutical composition for use in treating or preventing a disease in a subject in need thereof. In another aspect, the present disclosure provides a provided compound or pharmaceutical composition for use in treating a disease in a subject in need thereof. In another aspect, the present disclosure provides a provided compound or pharmaceutical composition for use in preventing a disease in a subject in need thereof.

In another aspect, the present disclosure provides a provided compound or pharmaceutical composition for use in the manufacture of a medicament for treatment or prevention of a disease in a subject in need thereof. In another aspect, the present disclosure provides a provided compound or pharmaceutical composition for use in the manufacture of a medicament for treatment of a disease in a subject in need thereof. In another aspect, the present disclosure provides a provided compound or pharmaceutical composition for use in the manufacture of a medicament for prevention of a disease in a subject in need thereof.

In certain embodiments, the disease is associated with QSOX1 mRNA (e.g., breast cancer (e.g., triple negative breast cancer)). In certain embodiments, the disease is a proliferative disease. In certain embodiments, the proliferative disease is cancer. In certain embodiments, the cancer is breast cancer (e.g., triple negative breast cancer). In certain embodiments, the cancer is triple negative breast cancer.

### Methods of Preparation

In another aspect, the present disclosure provides methods of preparing a compound of Formulae (II-a) or (II-b):

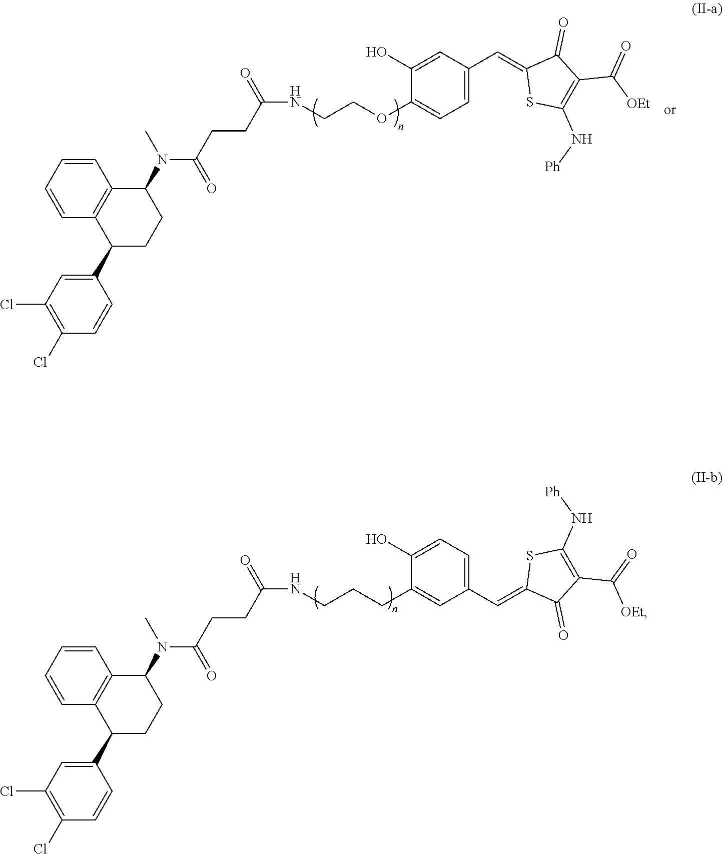

or a pharmaceutically acceptable salt thereof, comprising reacting a compound of Formulae (V-a) or (V-b):

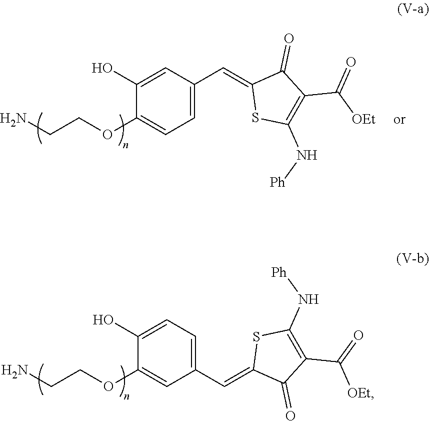

or a salt thereof, with a compound of Formula (VI):

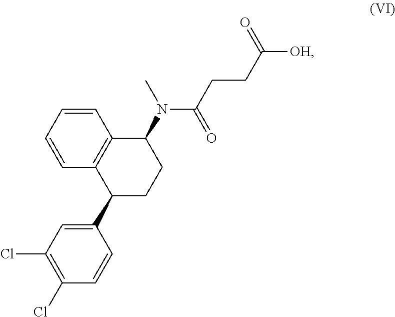 [SMILES: CN(C(=O)CCC(=O)O)[C@H]1CC[C@@H](c2ccc(Cl)c(Cl)c2)c2ccccc21]

or a salt thereof, wherein n is 0, 1, 2, 3, 4, 5, 6, 7, 8, 9, or 10. In certain embodiments, the method comprises reacting a compound of Formula (V-a), or a salt thereof, with a compound of Formula (VI), or a salt thereof, to prepare a compound of Formula (II-a). In certain embodiments, the method comprises reacting a compound of Formula (V-b), or a salt thereof, with a compound of Formula (VI), or a salt thereof, to prepare a compound of Formula (II-b).

In certain embodiments, the method further comprises alkylating a compound of Formula (VII):

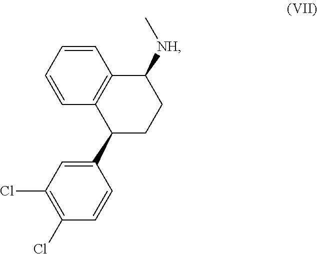 [SMILES: CN[C@H]1CC[C@@H](c2ccc(Cl)c(Cl)c2)c2ccccc21]

or a salt thereof, to provide the compound of Formula (VI), or salt thereof.

In certain embodiments, the method further comprises alkylating a compound of Formulae (VIII-a) or (VIII-b):

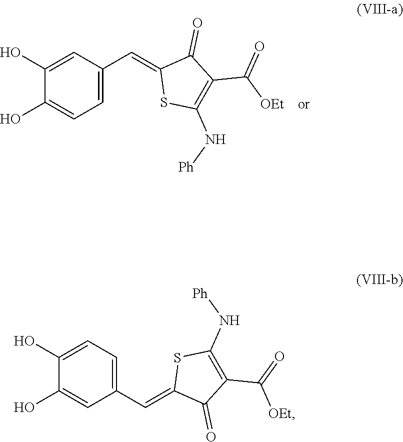 [SMILES: CCOC(=O)C1=C(Nc2ccccc2)S/C(=C\c2ccc(O)c(O)c2)C1=O ; CCOC(=O)C1=C(Nc2ccccc2)S/C(=C\c2ccc(O)c(O)c2)C1=O]

or a salt thereof, to provide the compound of Formulae (V-a) or (V-b):

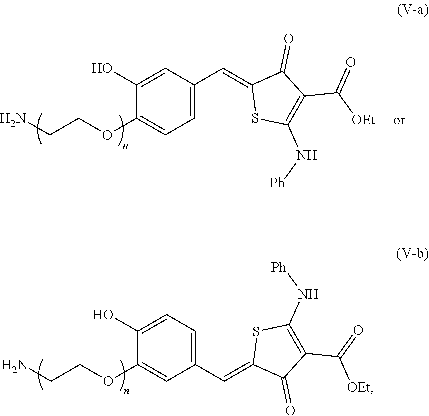

or a salt thereof. In certain embodiments, the method comprises alkylating a compound of Formula (VIII-a), or a salt thereof, to provide the compound of Formula (V-a). In certain embodiments, the method comprises alkylating a compound of Formula (VIII-b), or a salt thereof, to provide the compound of Formula (V-b).

As can be appreciated by the skilled artisan, methods of synthesizing the compounds of the formulae herein will be evident to those of ordinary skill in the art, including in the schemes and examples herein. Additionally, the various synthetic steps may be performed in an alternate sequence or order to give the desired compounds. In addition, the solvents, temperatures, reaction durations, etc. delineated herein are for purposes of illustration only and one of ordinary skill in the art will recognize that variation of the reaction conditions can produce the desired compounds of the present disclosure.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting in their scope.

### Example 1: Mapping Small Molecule Binding Sites Across the Human Transcriptome

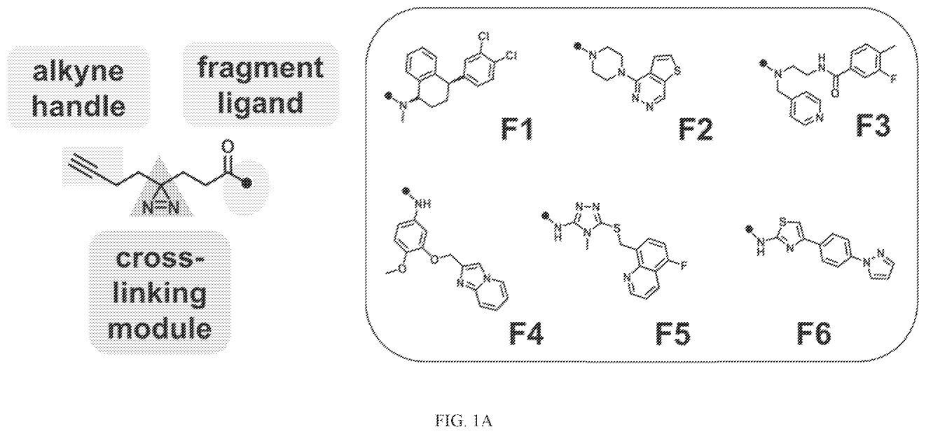

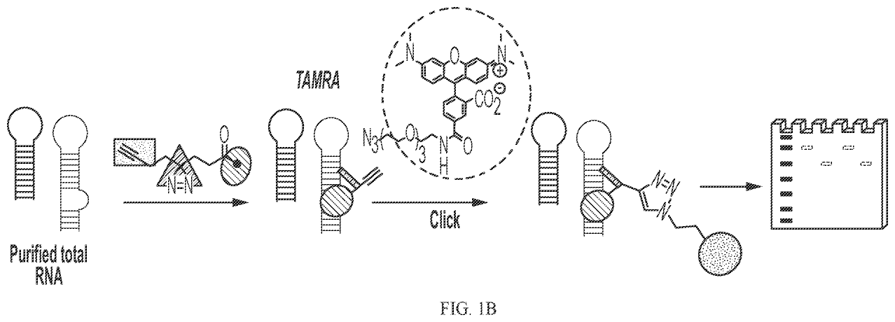

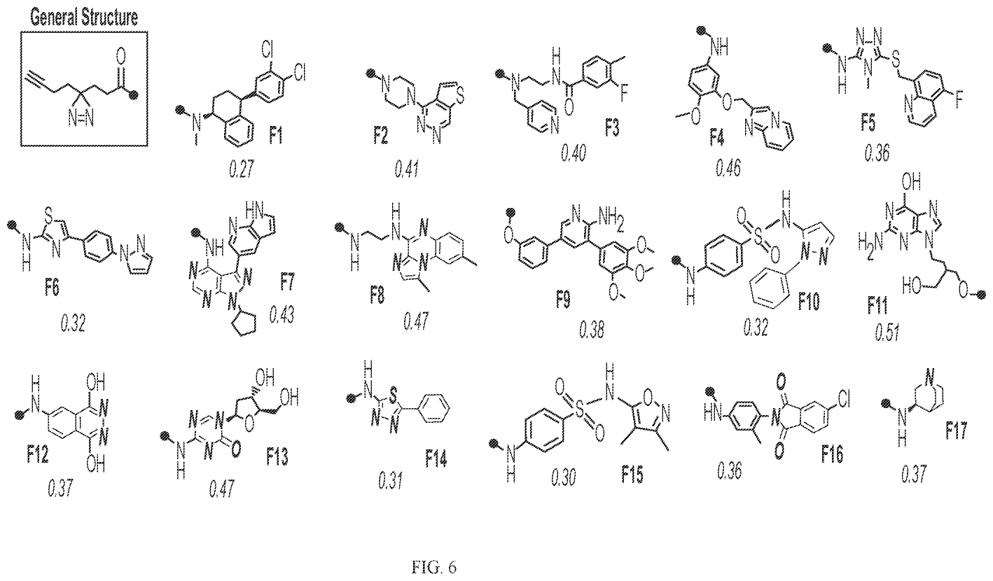

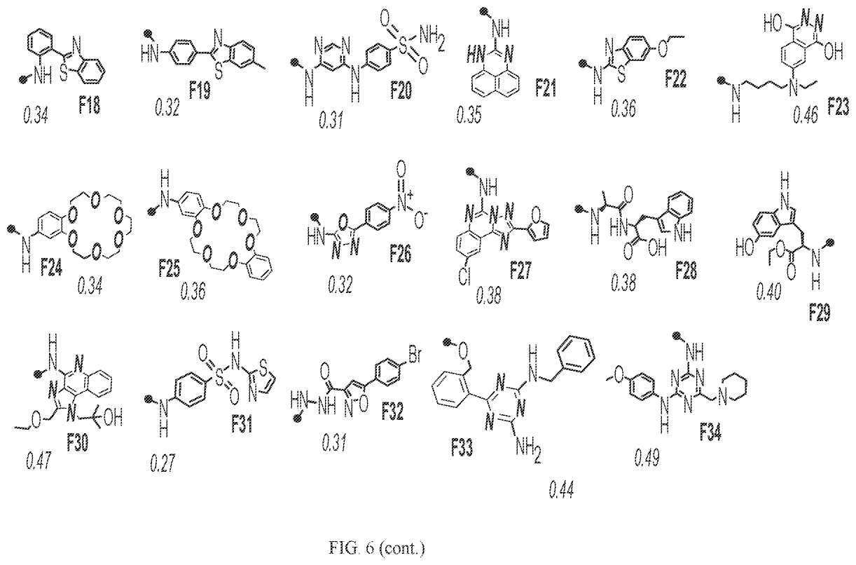

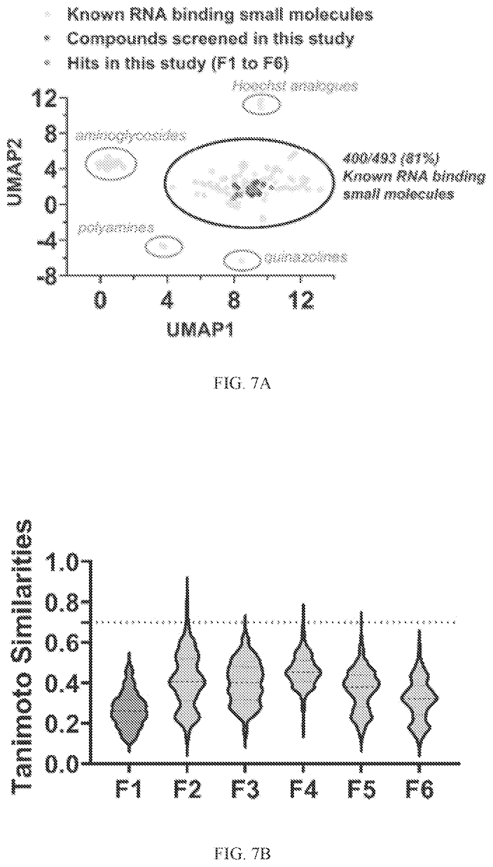

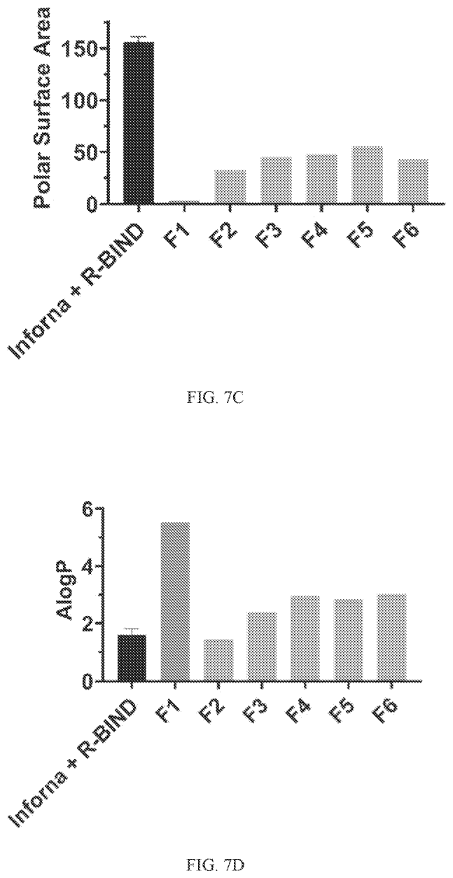

A panel of 34 structurally diverse compounds (FIG. 6) was appended with a diazirine moiety, which reacts with RNA upon photoactivation, and an alkyne tag (FIG. 1A). The alkyne tag can be clicked to an azide-functionalized fluorescent dye for imaging (FIG. 1B) or to azide-functionalized agarose beads for pull-down. A subset of the molecules were selected for their similarities to known RNA binders, as revealed by a two-dimensional Uniform Manifold Approximation and Projection (UMAP) analysis (FIG. 7A).¹⁴ The others were selected to include novel chemotypes not known to bind RNAs, including thienopyridazines and phenylpyrazoles (FIGS. 6 & 7A-7D).

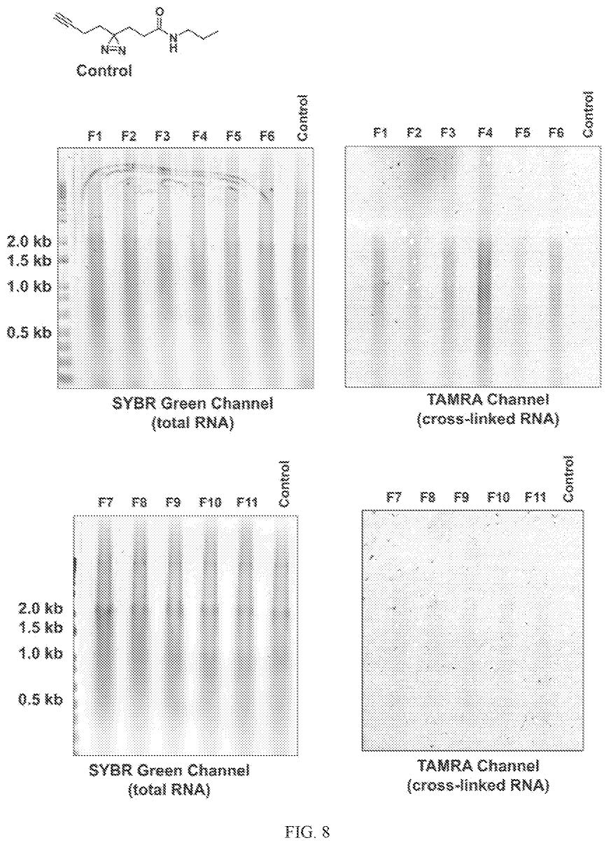

Compounds were first evaluated in vitro with total RNA harvested from MDA-MB-231 triple negative breast cancer (TNBC) cells. Following irradiation, the cross-linked RNAs, which then contained an alkyne handle, were clicked to the fluorescent dye tetramethylrhodamine (TAMRA) azide (FIG. 1B). Analysis of the samples by gel electrophoresis and fluorescence imaging identified six compounds (F1-F6; FIG. 1B) with reproducible cross-linking to the RNA targets, as defined by signal at least 3-fold above background. Based on the size of the bands, many of the cross-linked targets included mRNAs (FIG. 8).

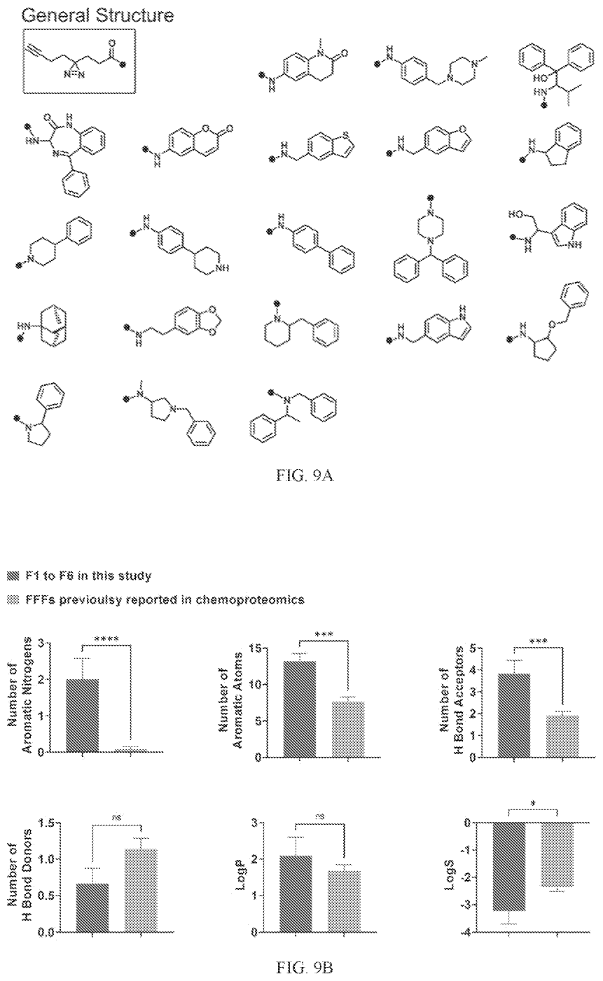

All six compounds contain aromatic rings, including nitrogen-rich aromatic heterocycles, such as pyridine, triazole, and N-methyl piperazine that are known to bind RNA. 15-17 F1, however, is unique as it has no aromatic nitrogen. When compared to the known RNA-binding molecules,^{18, 19} F1 is the most dissimilar to known RNA-binding compounds, with an average Tanimoto coefficient of 0.27±0.09 (FIG. 7B). The average Tanimoto coefficient for the other molecules indicates that they, too, are chemically dissimilar (0.32-0.46), although the range of four compounds include coefficients³ 0.7. F1 also has the lowest topological polar surface area of the compounds, all of which are significantly lower than known RNA binders (FIGS. 7B-7C). In contrast, when comparing atomic log P, only F1 is significantly different than known RNA binders (FIG. 7D). When compared to molecules previously reported to target protein in chemoproteomics studies,^{10, 20} RNA-targeting compounds of the present disclosure have a higher degree of aromaticity and have more H-bond acceptors (FIGS. 9A-9B).

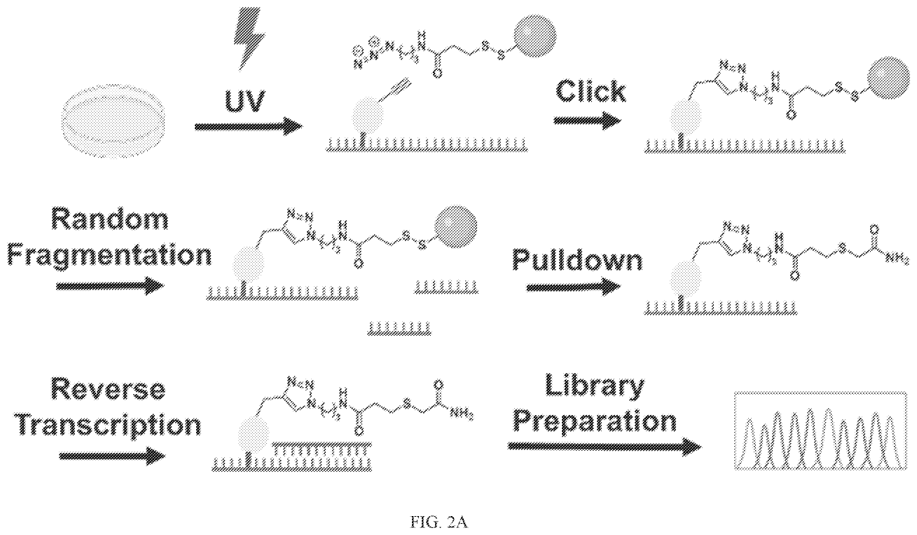

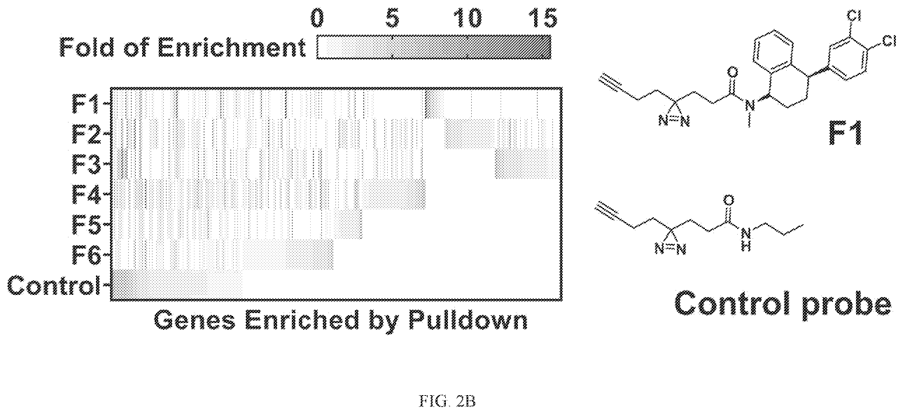

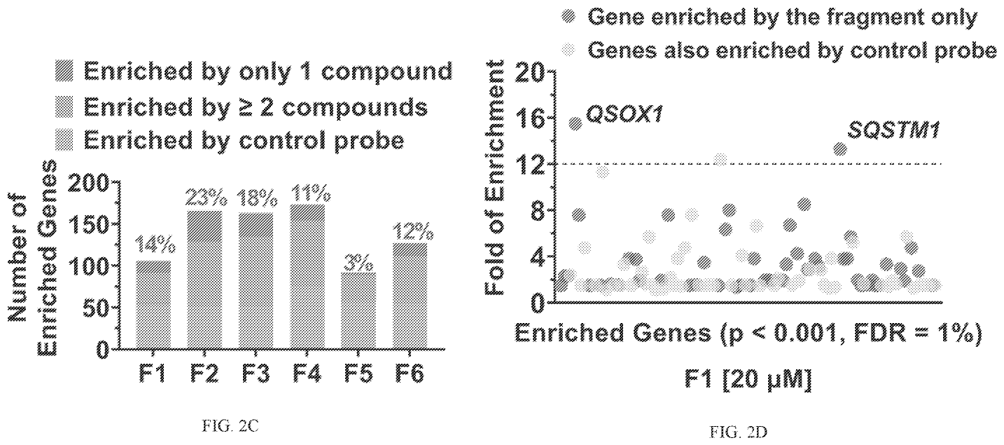

Additional studies were performed in live MDA-MB-231 cells to profile the cellular targets as well as the small molecule binding site within them, via Chem-CLIP-Map-Seq,²¹ which had not previously been applied transcriptome-wide. After treatment (20 μM; 16 h) and cross-linking, total RNA was harvested and fragmented prior to pull-down (FIG. 2A). Random fragmentation was completed first to: 1) reduce sequence bias in library preparation, 2) reduce background signal in the RNA-seq analysis; and 3) allow for only the regions bound by the small molecules to be pulled down rather than the entire transcript, identifying both the target and the binding site within it. In parallel, a control diazirine probe that lacks an RNA-binding functionality (FIG. 2B) was used to identify targets that non-specifically react with the cross-linking moiety, where cross-linking is not driven by the RNA-binding elements.

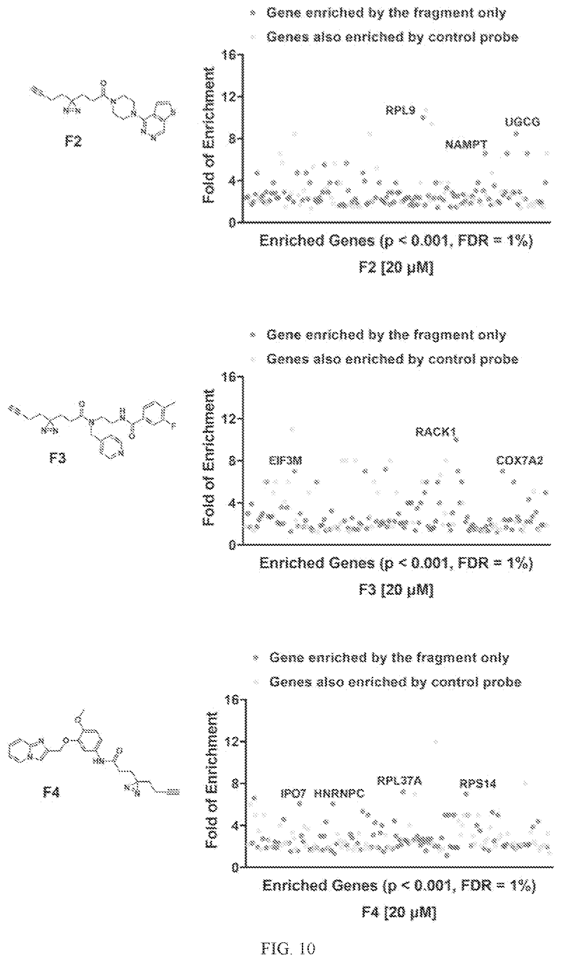

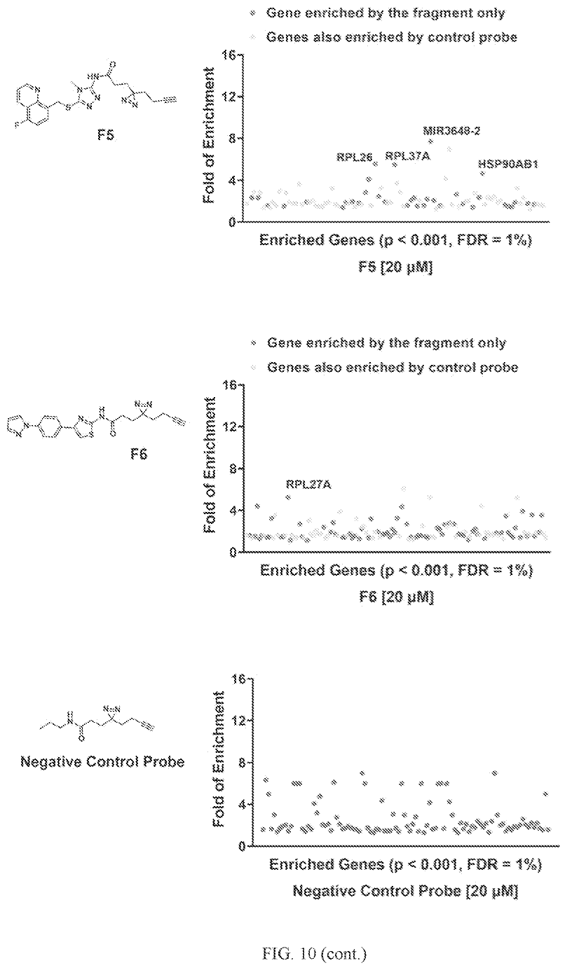

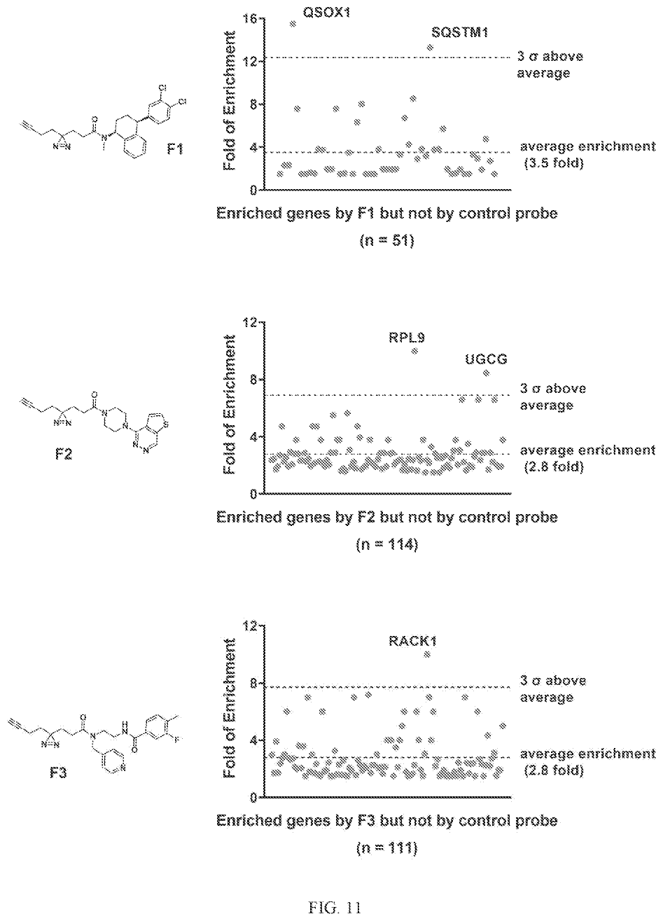

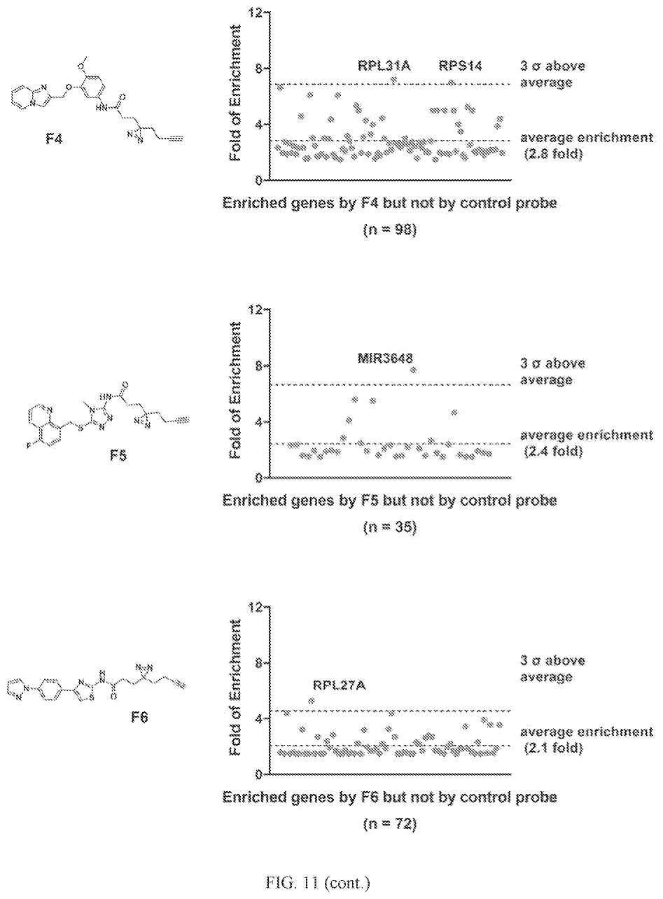

Significantly enriched regions across the human transcriptome (p<0.001) were identified by using a publicly available package, Genrich²² (FIGS. 2B, 10 & 11). Genrich uses a null model with a log-normal distribution to calculate p values by comparing sequencing runs of the same RNA sample before and after pull-down. Additional filters were applied to these significantly enriched regions, requiring a minimum read count of 5 and a minimum enrichment of 1.5, thereby eliminating low-confidence peaks. The distribution of the enrichment of RNAs bound by each molecule and the control probe is shown in FIGS. 2B, 9A-9B & 10.

Distinct patterns of target enrichment were observed for each compound when compared to each other and to the control probe (FIG. 2B). Unique transcripts were identified for each small molecule [n=3 (F5) to 38 (F2)] (FIG. 2C). After eliminating transcripts that were also bound by the control diazirine fold, such only bona fide targets remain, the number of transcripts enriched by F1 (n=51) and F5 (n=35) shows superior selectivity compared to other compounds (n>70) (FIG. 10). The largest enrichments were observed for F1, with two transcripts enriched >12-fold (FIG. 2D). The compounds also appear to have different distributions across transcript regions, i.e., 5′ and 3′ untranslated regions (UTR), coding regions (CDS), or introns, as well as noncoding RNAs (FIG. 12).

Next, the exact binding site within each transcript was mapped for all six molecules. Small molecules cross-linked to RNA can inhibit reverse transcriptase (RT) from proceeding near the cross-linked site, inducing an “RT stop” and resulting in a truncated cDNA strand.²¹ Thus, the termini of enriched sequences were used to map the binding site of compounds to each RNA target Some enriched regions have no discernable “RT stop” sites, suggesting that these regions may form flexible structures and lack a defined binding site.²³ Although mutations have also been observed in RNA-protein cross-linking studies,^{24, 25} no significant enrichment of mutations were observed in the present disclosure. This is likely due to the bulky size of cross-linked compounds compared to that of an amino acid residue after proteolysis in RNA-protein cross-linking studies.

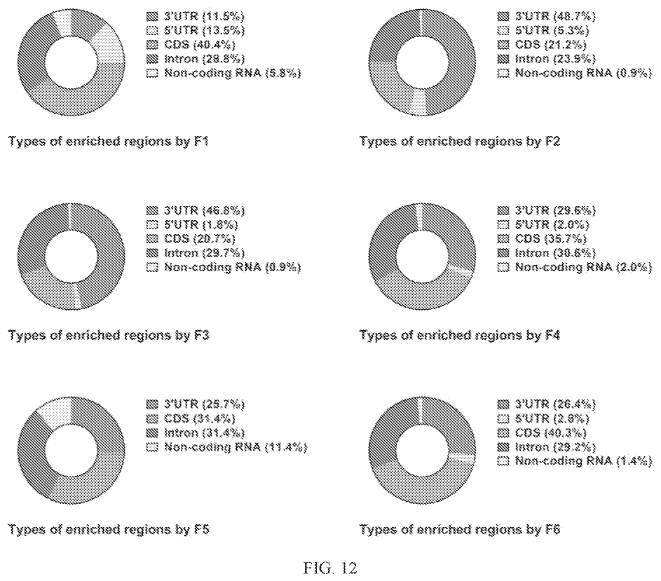

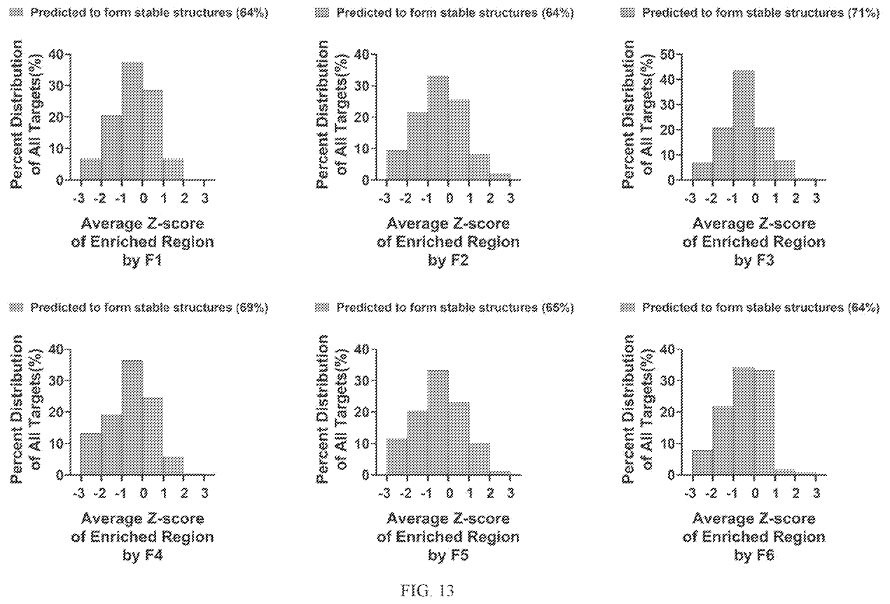

Identifying the sites occupied by a small molecule could allow direct experimental inference of RNA structure in cells. The structure of each transcript was therefore modeled at the small molecule binding site by using the state-of-the-art program ScanFold, which calculates the minimum free energy of the native sequence and compares it to that of random sequences to identify unusually stable structures within an RNA.²⁶ Over 60% of enriched regions are predicted to form stable, ordered structures (FIG. 13).

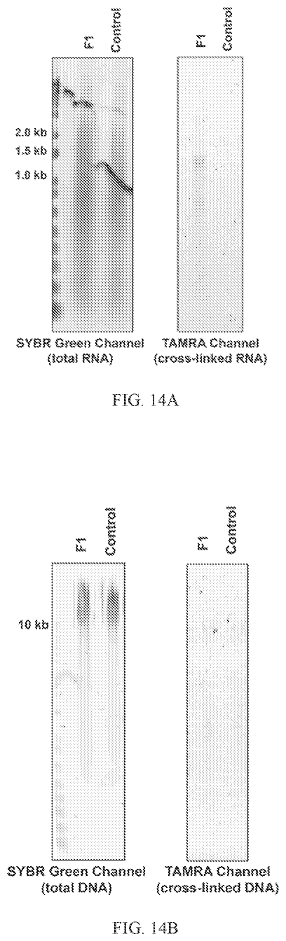

F1 was further investigated, as it is a novel RNA-binding chemotype, binds the second fewest number of transcripts, and has the highest fold of enrichment of a transcript-12-fold for two targets that are also unique to F1, QSOX1 and SQSTM1 (Sequestosome 1) (FIGS. 2D & 11). It was first determined whether other biomolecules were pulled down by F1, as the diazirine could also cross-link to DNA or protein. No significant enrichment of protein or DNA targets was observed for F1 when compared to the control probe, suggesting that it preferentially engages RNAs in cells (FIGS. 14A-14D). In agreement with these data, full proteomics analysis of proteins cross-linked to F1 showed that only 79 proteins of the 3,158 proteins detectable before pull-down were selectively pulled-down by F1, i.e., they were not pulled-down by the control probe. None of these proteins are known to form mRNA-protein complexes, nor were QSOX1 or SQSTM1 pulled down.

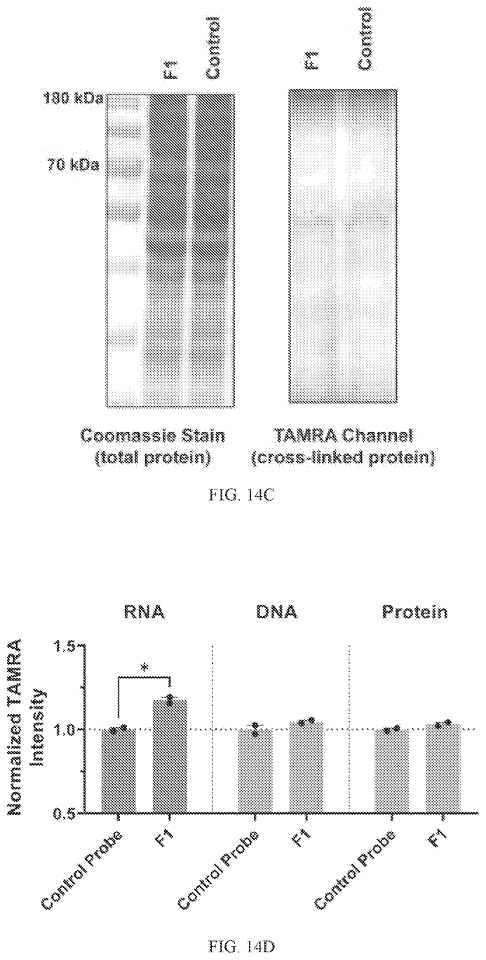

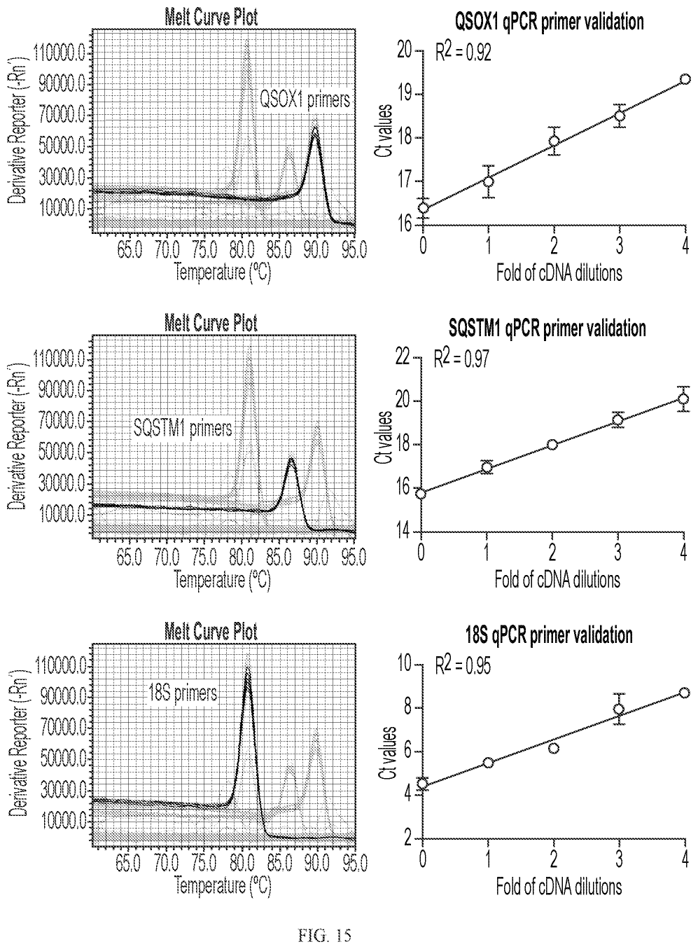

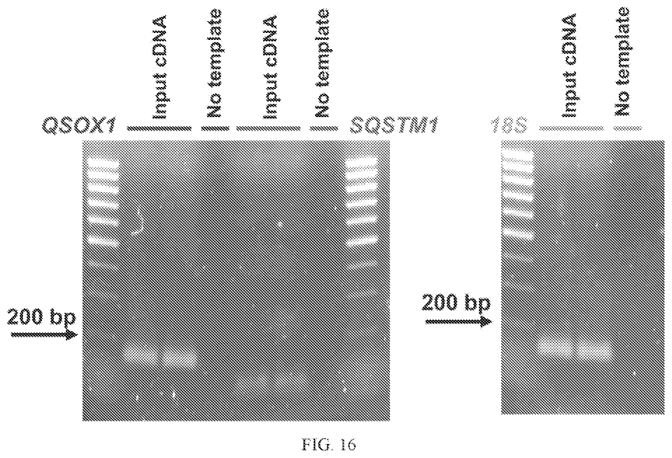

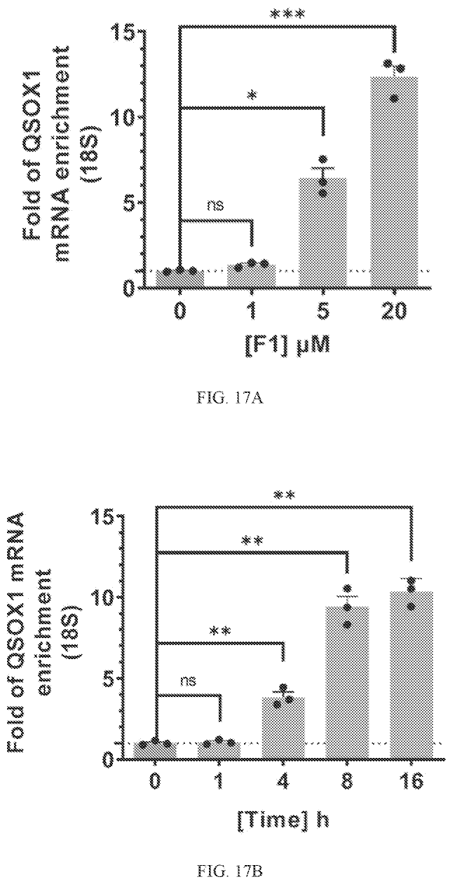

The time and dose dependence of the pull-down of QSOX1 mRNA by F1 were also studied by using RT-qPCR (see FIGS. 15-16 for primer validation). Compound F1 dose-dependently enriched QSOX1 mRNA, at 5 μM (˜6-fold) and 20 μM (˜12-fold)(16 h incubation; FIG. 17A). The pull-down of OSOX1 mRNA was also time dependent. After a 1 h incubation, no enrichment of the QSOX1 was observed. A ˜4-fold enrichment was observed after 4 h, with enrichment plateauing at 8 h and 16 h time points (FIG. 17B). The time required to engage and cross-link to the target is likely due to a composite of factors and could include dissociation of proteins bound to the RNA, the association rate of the small molecule, the rate of the RNA's folding, and/or structural dynamics.

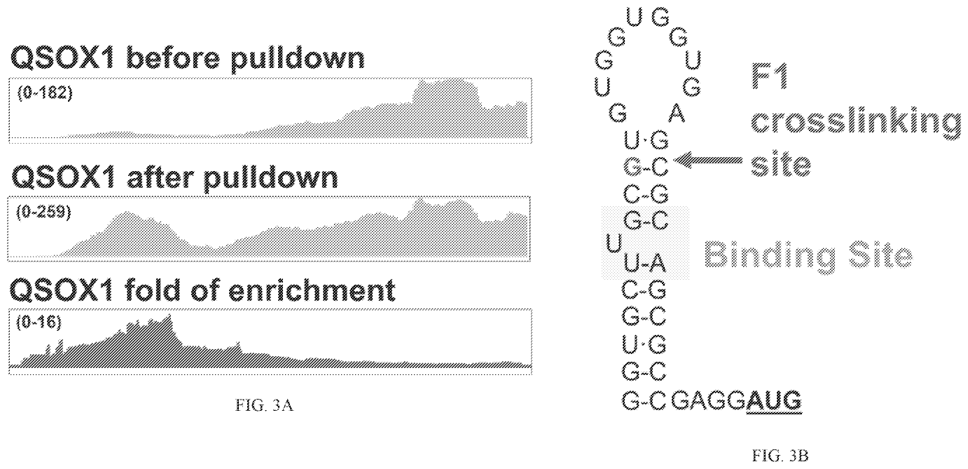

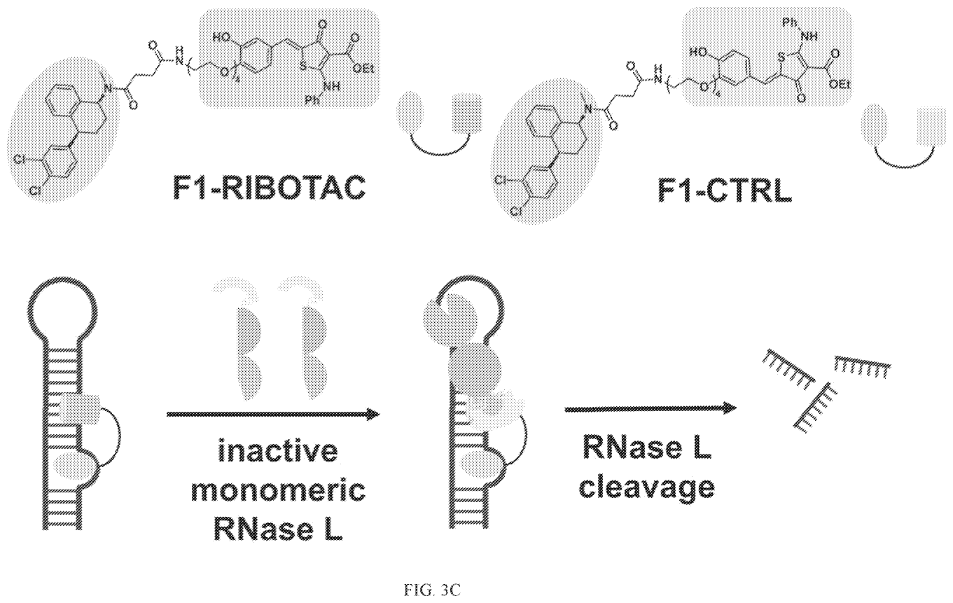

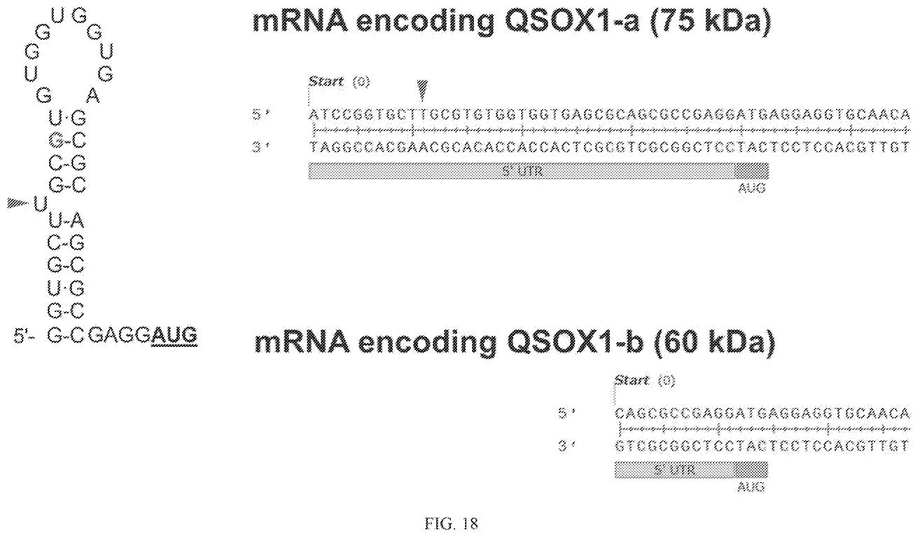

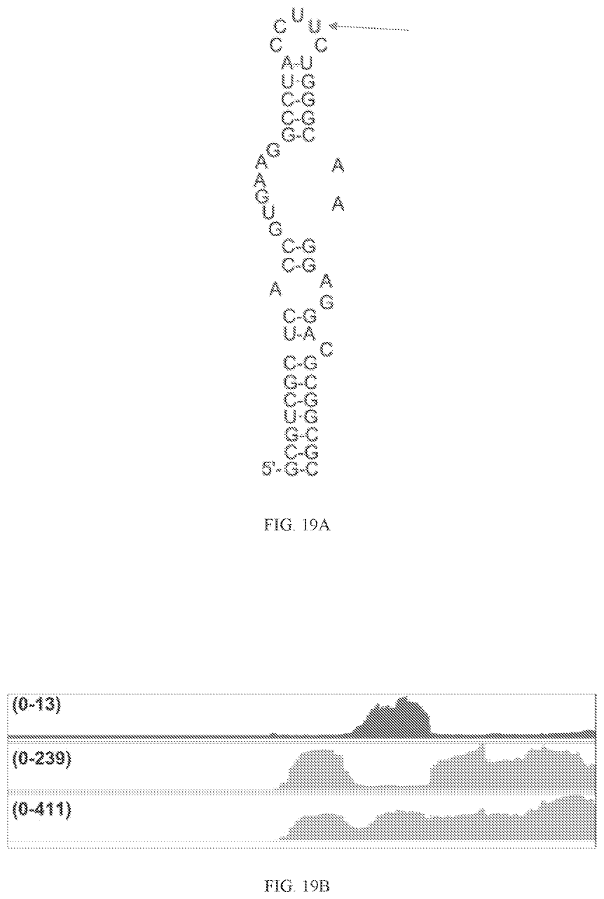

Analysis of the RNA-seq data coupled with ScanFold structure prediction enabled mapping of the F1 binding site within the QSOX1 and SOSTM1, the 5′ UTR of the former and the coding region of SQSTM1. The putative binding site in QSOX1 is predicted to form a stable hairpin structure containing a single bulged uridine (FIGS. 3A-3B), the site of an “RT Stop” in the RNA-seq data. The sequence that forms this structure is present only in the QSOX1-a isoform and not in the QSOX1-b isoform (FIG. 18). The SQSTM binding site also forms a structure, a hairpin with single bulged cytosine and two asymmetric internal loops. In SOSTM, the “RT Stop” occurs at a uridine residue in the hairpin loop (FIGS. 19A-19B).

Since it is unknown whether these targetable structures in QSOX1-a or SOSTM1 are functional, the regions surrounding the binding sites were examined for potential sites sensitive to RNase L cleavage to enact a targeted degradation strategy. This strategy, which uses ribonuclease-targeting chimeras (RIBOTACs) comprising an RNA-binding module and an RNase L-recruiting module, has been advantageous for other RNA targets, as RIBOTACs are catalytic, sub-stoichiometric, more selective, and more potent.²⁷ RNase L, mainly localized in cytoplasm, preferentially cleaves RNA with UNN pattern (unpaired uridines).²⁸⁻³⁰ Three such sites are embedded in a hairpin adjacent to the F1 binding site in the QSOX1-a mRNA (FIG. 3B). Only one such site is nearby the SQSTM binding site and at greater than twice the distance (6 vs. 13 nucleotides; FIGS. 19A-19B). It was therefore hypothesized that functional activity and selectivity could be engineered into F1 towards QSOX1 (vs. SQSTM1) by converting it into a RIBOTAC.

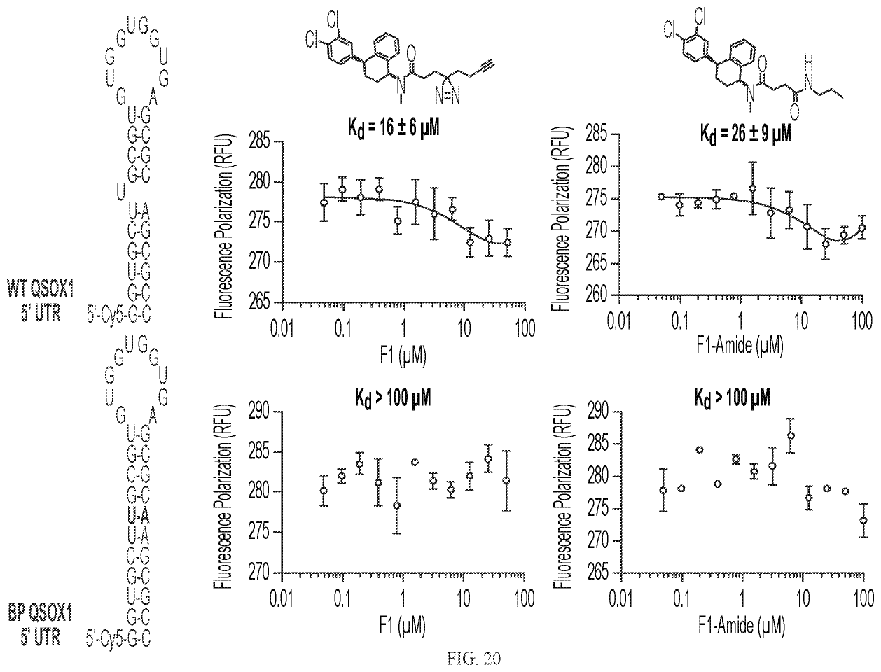

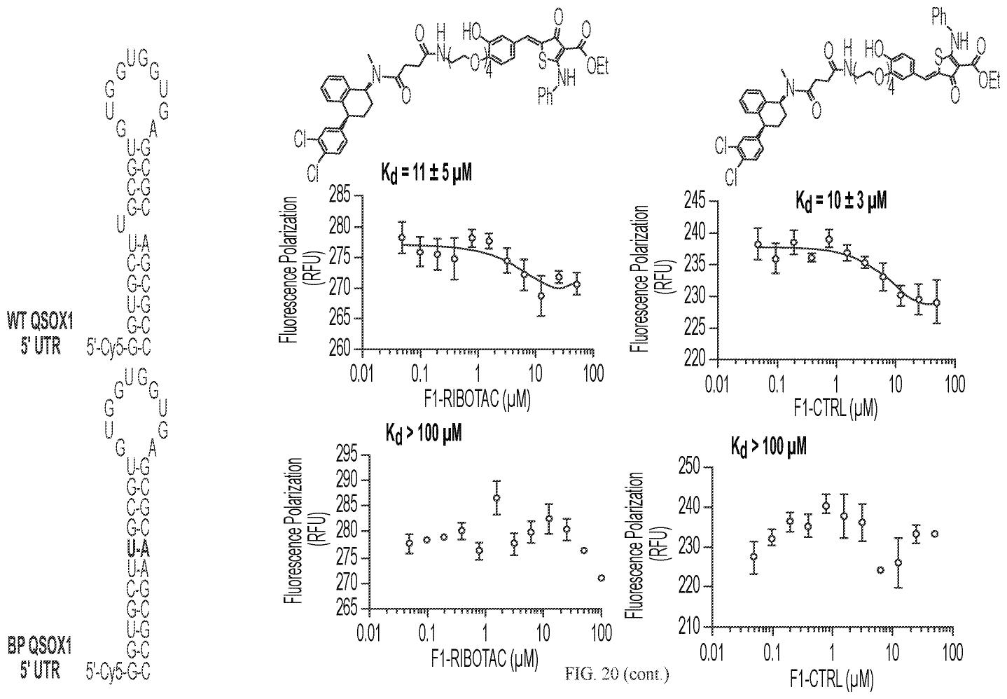

The binding affinity of four molecules to a model of the QSOX1-a binding site was studied by fluorescence polarization with Cy5-labeled RNA: F1, F1-Amide, F1-RIBOTAC, and F1-CTRL (FIGS. 3C & 20). F1-Amide mimics the conjugation of the RNase L recruiting module while F1-CTRL is a control RIBOTAC that uses an RNase L recruiting module with >20-fold reduced activity.³¹ That is, F1-Amide and F1-CTRL are expected to bind with similar affinity, and have similar cellular activity and the same mode of action—binding—as F1 does. F1 binds to the bulged U nucleotide in the QSOX1 mRNA with a K_{d} of 16±6 mM; however, no detectable binding to a fully paired RNA in which the U bulge was converted to an AU base pair was observed (FIG. 20). F1-Amide (K_{d}=26±9 UM), F1-RIBOTAC (K_{d}=11±5 μM), and F1-CTRL (K_{d}=10±3 μM) bind with similar affinity as F1 (FIG. 20). Additionally, no measurable binding was observed for all three compounds to a mutant RNA lacking U-bulge (K_{d}>100 μM) (FIG. 20).

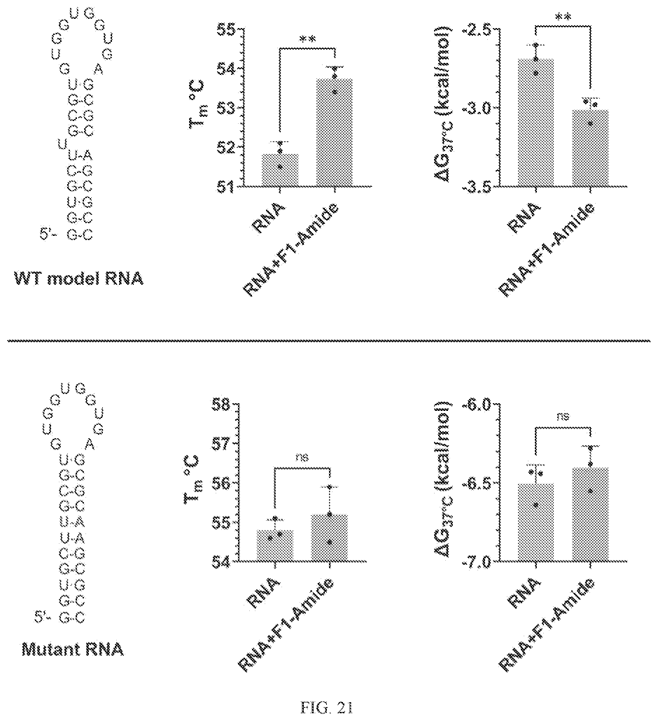

Two additional experiments were used to verify these binding data. Optical melting was used to study whether the binding of F1-Amide stabilized the folding of the RNA. The melting temperature (Tₘ) increased by 1.9° C. and the DG^{o}₃₇ decreased by 0.3 kcal/mol upon addition of small molecule (FIG. 21). No changes in the Tₘ or DG^{o}₃₇ were observed when the mutant RNA was melted in the presence or absence of F1 (FIG. 21). Binding was also studied by using in vitro Chem-CLIP, where F1 selectively cross-linked to a model of the QSOX1-a 5′ UTR hairpin. Cross-linking of F1 was ablated by mutating the U-bulge into an A/U base pair, supporting that F1 binds to the U-bulge that is three nucleotides upstream of the mapped cross-linking site (FIGS. 3B & 22A). Competitive Chem-CLIP further confirmed the binding of F1-Amide and F1-RIBOTAC on the same site with F1, as they dose-dependently competed off the cross-linking of F1 to the QSOX1-a target site in vitro (FIG. 22B).

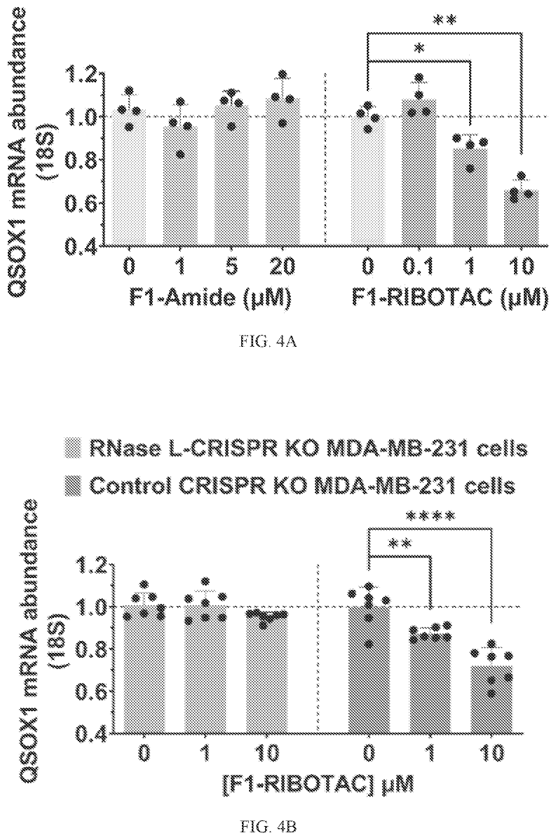

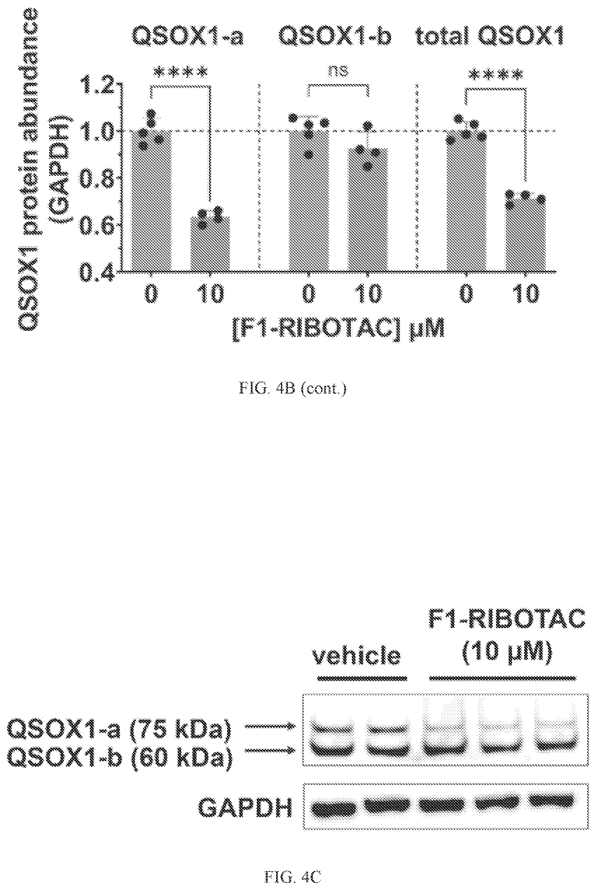

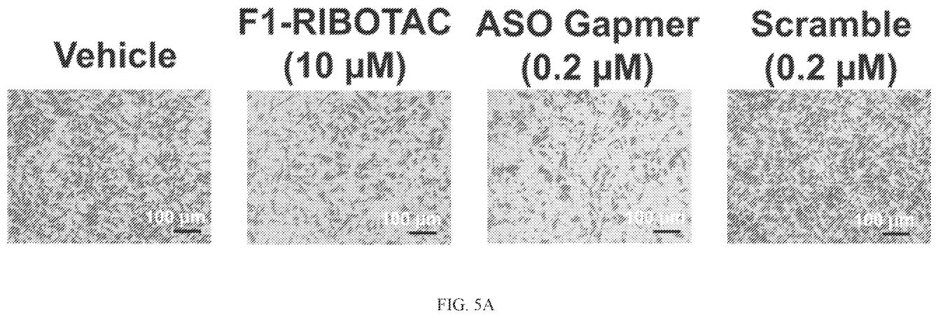

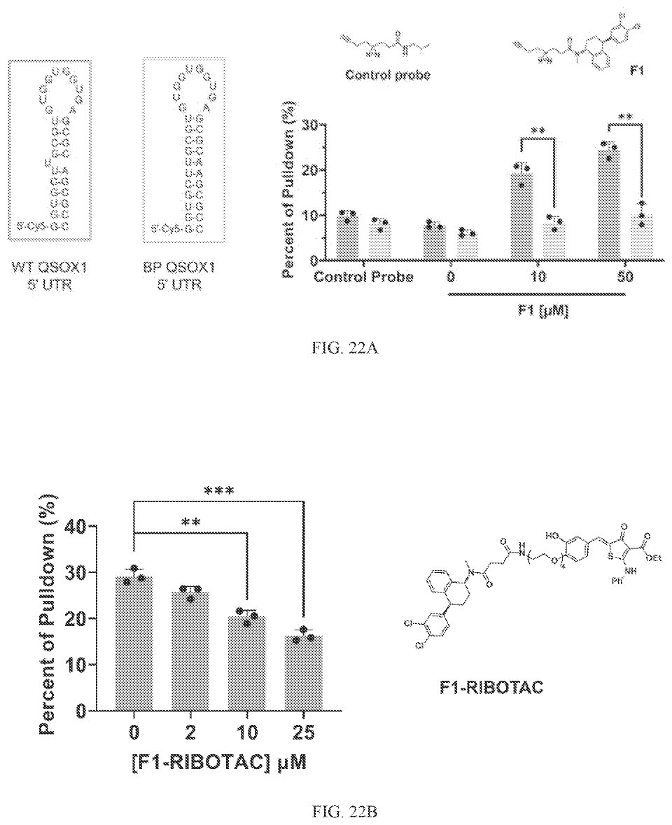

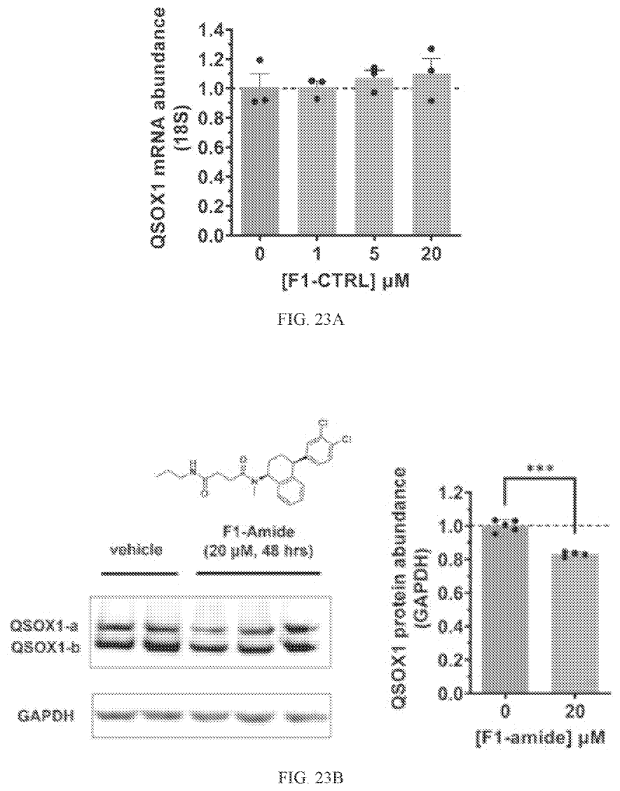

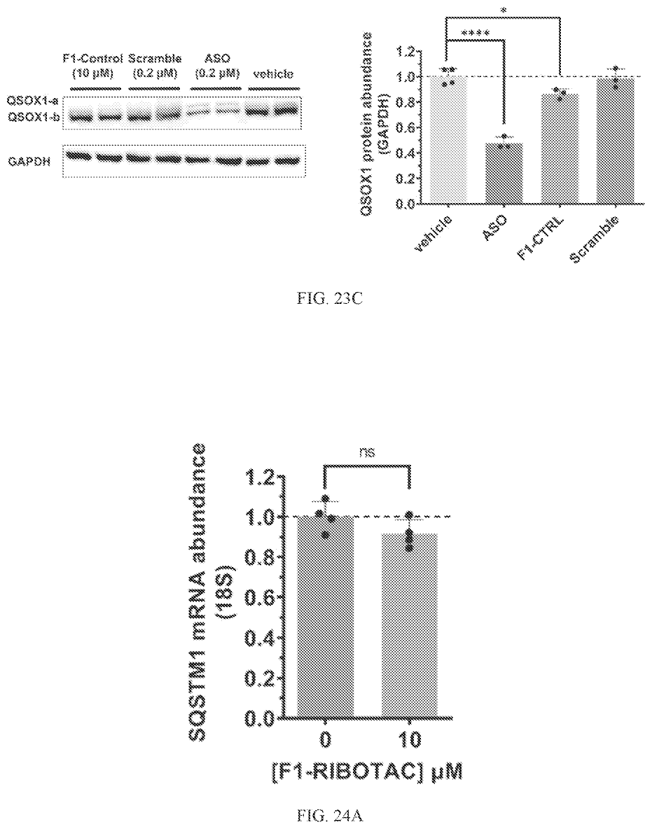

QSOX1 is an oncogene that promotes invasion and proliferation of breast cancer cells.^{32, 33} In MDA-MB-231 TBNC cells, F1-RIBOTAC dose-dependently decreased QSOX1-a mRNA levels up to ˜35% at the 10 mM, the highest dose with no effect on viability (FIG. 4A). Neither F1-Amide nor F1-CTRL affected QSOX1-a mRNA levels (FIG. 23A), suggesting F1-RIBOTAC cleaves target RNA by recruiting and activating RNase L. This mode of action was further validated by treating F1-RIBOTAC in CRISPR-edited cells with RNase L knockdown. The effect of F1-RIBOTAC on QSOX1-a mRNA was ablated in RNase L-knockdown cells but not in control cells, confirming that F1-RIBOTAC decreases QSOX1 mRNA in an RNase L-dependent manner (FIG. 4B).

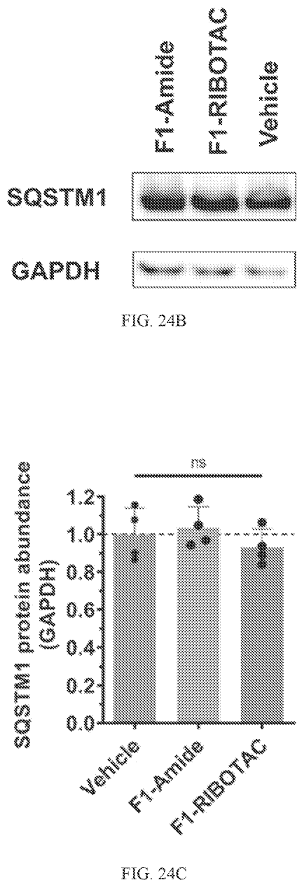

No significant effect was observed on SOSTM1 mRNA levels upon treatment with F1-RIBOTAC (FIG. 24A). As F1 enriched QSOX1 and SOSTM1 similarly, this difference in the RIBOTAC's activity is likely due to differences in the structure and sequence around the binding site, i.e., a single distal unpaired U in SOSTM1, rendering it a poor substrate for RNase L.

At the protein level, only QSOX1-a abundance was reduced by F1 RIBOTAC treatment, by ˜35% at 10 μM (FIG. 4C), and no effect was observed on QSOX-1b levels, indicating that F1 RIBOTAC is isoform-specific. Previously, a small molecule was developed that targets both QSOX1 protein isoforms.³³ Therefore, F1-RIBOTAC could be useful to study the individual biological roles of each isoform by exploiting the structural differences in their encoding mRNAs.

In contrast to the results described above for F1 RIBOTAC, only ˜15% reduction of QSOX1-a protein was observed by treatment with 20 μM of F1-Amide or F1-CTRL (FIGS. 23B-23C). These results in conjunction with the lack of effect on mRNA abundance, suggest that: (i) F1-Amide and F1-CTRL bind the QSOX1-a 5′ UTR and impede translation by presumably blocking ribosomal assembly or processivity, as has been described for other compounds targeting RNA;³⁴ and (ii) the QSOX1-a binding site is indeed functional. Neither F1-Amide nor F1-RIBOTAC had any effect on SQSTM1 protein levels (FIGS. 24B-24C), the former suggesting that F1's binding within SQSTM1 is biologically silent.

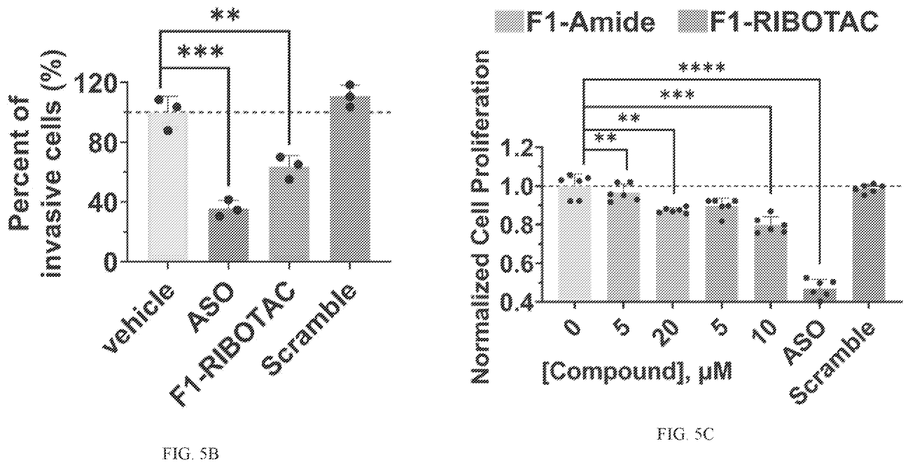

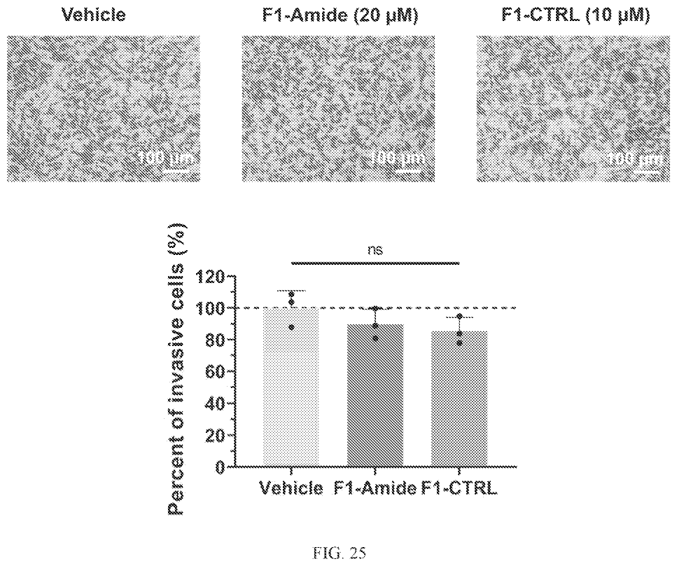

It was next assessed whether the isoform-specific reduction of QSOX1-a protein by F1-RIBOTAC was sufficient to inhibit QSOX1-mediated phenotypes. 10 μM of F1-RIBOTAC significantly reduced the number of invasive MDA-MB-231 cells, by ˜40% (FIGS. 5A-5B). In contrast, no significant effect was observed by F1-Amide or F1-CTRL (FIG. 25). In a proliferation assay, both F1-Amide and F1-RIBOTAC dose-dependently reduced proliferation of MDA-MB-231 cells, with F1-RIBOTAC showing enhanced activity compared to F1-Amide delivered at twice the dose (FIG. 5C). Collectively, these results suggest that converting F1-Amide to F1-RIBOTAC improved its potency in reverting disease-related phenotypes.

The methods of the present disclosure map small molecule binding sites across the human transcriptome in live cells. These methods can be used to study the molecular recognition patterns between RNA and ligands in an unbiased way in intact biological systems. Additionally, they can also be applied to inferring RNA structures mediated by RNA alone or by RNA-protein complexes that would be otherwise difficult to detect.

### REFERENCES

1. Ding, Y.; Tang, Y.; Kwok, C. K., Zhang, Y., Bevilacqua, P. C.; Assmann, S. M., In vivo genome-wide profiling of RNA secondary structure reveals novel regulatory features. Nature 2014, 505 (7485), 696-700
2. Wan, Y.; Kertesz, M.; Spitale, R. C.; Segal, E.; Chang, H. Y., Understanding the transcriptome through RNA structure. Nat. Rev. Genet. 2011, 12 (9), 641-655.
3. Strobel, E. J.; Yu, A. M.; Lucks, J. B., High-throughput determination of RNA structures. Nat. Rev. Genet. 2018, 19 (10), 615-634.
4. Spitale, R. C.; Flynn, R. A.; Zhang, Q. C.; Crisalli, P.; Lee, B.; Jung, J. W.; Kuchelmeister, H. Y.; Batista, P. J.; Torre, E. A.; Kool, E. T.; Chang, H. Y., Structural imprints in vivo decode RNA regulatory mechanisms. Nature 2015, 519 (7544), 486-90.
5. Ratni, H.; Ebeling, M.; Baird, J.; Bendels, S.; Bylund, J.; Chen, K. S.; Denk, N.; Feng, Z.; Green, L.; Guerard, M.; Jablonski, P.; Jacobsen, B.; Khwaja, O.; Kletzl, H.; Ko, C. P.; Kustermann, S.; Marquet, A.; Metzger, F.; Mueller, B.; Naryshkin, N. A.; Paushkin, S. V.; Pinard, E.; Poirier, A.; Reutlinger, M.; Weetall, M.; Zeller, A., Zhao, X.; Mueller, L., Discovery of Risdiplam, a selective survival of motor neuron-2 (SMN2) gene splicing modifier for the treatment of spinal muscular atrophy (SMA). J. Med. Chem. 2018, 61(15), 6501-6517.
6. Disney, M. D., Targeting RNA with small molecules to capture opportunities at the intersection of chemistry, biology, and medicine. J. Am. Chem. Soc. 2019, 141(17), 6776-6790.
7. Guan, L.; Disney, M. D., Covalent small molecule-RNA complex formation enables cellular profiling of small molecule-RNA interactions. Angew. Chem. Int. Ed. Engl. 2013, 52 (38), 10010-3.
8. Balaratnam, S.; Rhodes, C.; Bume, D. D.; Connelly, C.; Lai, C. C.; Kelley, J. A.; Yazdani, K., Homan, P. J., Incarnato, D.; Numata, T.; Schneekloth, J. S., Jr., A chemical probe based on the PreQ1 metabolite enables transcriptome-wide mapping of binding sites. Nat. Commun. 2021, 12(1), 5856
9. Wang, J.; Schultz, P. G.; Johnson, K. A., Mechanistic studies of a small-molecule modulator of SMN2 splicing. Proc. Natl. Acad. Sci. U.S.A 2018, 115(20), E4604-e4612.
10. Parker, C. G.; Galmozzi, A., Wang, Y.; Correia, B. E.; Sasaki, K.; Joslyn, C. M.; Kim, A. S.; Cavallaro, C. L.; Lawrence, R. M.; Johnson, S. R.; Narvaiza, I.; Saez, E.; Cravatt, B. F., Ligand and target discovery by fragment-based screening in human cells. Cell 2017, 168 (3), 527-541.e29.
11. Spradlin, J. N.; Zhang, E.; Nomura, D. K., Reimagining druggability using chemoproteomic platforms. Acc. Chem. Res. 2021, 54(7), 1801-1813.
12. Zhang, X.; Spiegel, J.; Martinez Cuesta, S.; Adhikari, S.; Balasubramanian, S., Chemical profiling of DNA G-quadruplex-interacting proteins in live cells. Nat. Chem. 2021, 13 (7), 626-633.
13. Di Antonio, M.; McLuckie, K. I.; Balasubramanian, S., Reprogramming the mechanism of action of chlorambucil by coupling to a G-quadruplex ligand. J. Am. Chem. Soc. 2014, 136 (16), 5860-3.
14. McInnes, L.; Healy, J.; Melville, J., Umap: Uniform manifold approximation and projection for dimension reduction. Submitted on 2018 Feb. 9. ar Xiv preprint: 1802.03426. https://doi.org/10.48550/arXiv.1802.03426. (accessed 2022 Jan. 9).
15. Carnevali, M.; Parsons, J.; Wyles, D. L.; Hermann, T., A modular approach to synthetic RNA binders of the hepatitis C virus internal ribosome entry site. Chembiochem 2010, 11 (10), 1364-7.
16. Hilimire, T. A.; Chamberlain, J. M.; Anokhina, V.; Bennett, R. P.; Swart, O.; Myers, J. R.; Ashton, J. M.; Stewart, R. A., Featherston, A. L.; Gates, K.; Helms, E. D.; Smith, H. C.; Dewhurst, S.; Miller, B. L., HIV-1 frameshift RNA-targeted triazoles inhibit propagation of replication-competent and multi-drug-resistant HIV in human cells. ACS. Chem. Biol. 2017, 12 (6), 1674-1682.
17. Di Giorgio, A.; Tran, T. P.; Duca, M., Small-molecule approaches toward the targeting of oncogenic miRNAs: roadmap for the discovery of RNA modulators. Future Med. Chem. 2016, 8 (7), 803-16.
18. Disney, M. D.; Winkelsas, A. M.; Velagapudi, S. P.; Southern, M.; Fallahi, M.; Childs-Disney, J. L., Inforna 2.0: A platform for the sequence-based design of small molecules targeting structured RNAs. ACS. Chem. Biol. 2016, 11 (6), 1720-8.
19. Morgan, B. S.; Sanaba, B. G.; Donlic, A., Karloff, D. B.; Forte, J. E.; Zhang, Y.; Hargrove, A. E., R-BIND: an interactive database for exploring and developing RNA-targeted chemical probes. ACS. Chem. Biol. 2019, 14 (12), 2691-2700.
20. Wang, Y. J.; Dix, M. M.; Bianco, G.; Remsberg, J. R.; Lec, H. Y.; Kalocsay, M.; Gygi, S. P.; Forli, S., Vite, G.; Lawrence, R. M.; Parker, C. G.; Cravatt, B. F., Expedited mapping of the ligandable proteome using fully functionalized enantiomeric probe pairs. Nat. Chem. 2019, 11 (12), 1113-1123.
21. Velagapudi, S. P.; Li, Y.; Disney, M. D., A cross-linking approach to map small molecule-RNA binding sites in cells. Bioorg. Med. Chem. Lett. 2019, 29 (12), 1532-1536.
22. https://github.com/jsh58/Genrich [acesssed 2022 Jan. 9]
23. Mukherjee, H.; Blain, J. C.; Vandivier, L. E.; Chin, D. N.; Friedman, J. E.; Liu, F.; Maillet, A.; Fang, C., Kaplan, J. B.; Li, J., Chenoweth, D. M.; Christensen, A. B., Petersen, L. K.; Hansen, N. J. V.; Barrera, L.; Kubica, N.; Kumaravel, G.; Petter, J. C., PEARL-seq: a photoaffinity platform for the analysis of small molecule-RNA interactions. ACS. Chem. Biol. 2020, 15 (9), 2374-2381.
24. Linder, B.; Grozhik, A. V.; Olarerin-George, A. O.; Meydan, C.; Mason, C. E.; Jaffrey, S. R., Single-nucleotide-resolution mapping of m6A and m6Am throughout the transcriptome. Nat. Methods 2015, 12 (8), 767-72.
25. Hafner, M.; Katsantoni, M.; Köster, T.; Marks, J.; Mukherjee, J.; Staiger, D.; Ule, J.; Zavolan, M., CLIP and complementary methods. Nat. Rev. Dis. Primers 2021, 1(1), 20.
26. Andrews, R. J.; Roche, J.; Moss, W. N., ScanFold: an approach for genome-wide discovery of local RNA structural elements-applications to Zika virus and HIV. PeerJ 2018, 6, e6136.
27. Zhang, P.; Liu, X.; Abegg, D.; Tanaka, T.; Tong, Y.; Benhamou, R. I.; Baisden, J.; Crynen, G.; Meyer, S. M.; Cameron, M. D.; Chatterjee, A. K.; Adibekian, A.; Childs-Disney, J. L.; Disney, M. D., Reprogramming of protein-targeted small-molecule medicines to RNA by ribonuclease recruitment. J. Am. Chem. Soc. 2021, 143 (33), 13044-13055.
28. Han, Y.; Donovan, J.; Rath, S.; Whitney, G.; Chitrakar, A.; Korennykh, A., Structure of human RNase L reveals the basis for regulated RNA decay in the IFN response. Science 2014, 343 (6176), 1244-8.
29. Wreschner, D. H.; McCauley, J. W.; Skehel, J. J.; Kerr, I. M., Interferon action—sequence specificity of the ppp(A2′p)nA-dependent ribonuclease. Nature 1981, 289 (5796), 414-7.
30. Floyd-Smith, G.; Slattery, E.; Lengyel, P., Interferon action: RNA cleavage pattern of a (2′-5′) oligoadenylate—dependent endonuclease. Science 1981, 212 (4498), 1030-2.
31. Costales, M. G.; Aikawa, H.; Li, Y.; Childs-Disney, J. L., Abegg, D.; Hoch, D. G.; Pradeep Velagapudi, S.; Nakai, Y.; Khan, T.; Wang, K. W.; Yildirim, I.; Adibekian, A.; Wang, E. T.; Disney, M. D., Small-molecule targeted recruitment of a nuclease to cleave an oncogenic RNA in a mouse model of metastatic cancer. Proc. Natl. Acad. Sei. U.S.A. 2020, 117 (5), 2406-2411
32. Baek, J. A.; Song, P. H.; Ko, Y.; Gu, M. J., High expression of QSOX1 is associated with tumor invasiveness and high grades groups in prostate cancer. Pathol. Res. Pract. 2018, 214 (7), 964-967.
33. Fifield, A. L.; Hanavan, P. D.; Faigel, D. O.; Sergienko, E.; Bobkov, A.; Meurice, N.; Petit, J. L.; Polito, A.; Caulfield, T. R.; Castle, E. P., Copland, J. A.; Mukhopadhyay, D.; Pal, K.; Dutta, S. K.; Luo, H.; Ho, T. H.; Lake, D. F., Molecular inhibitor of QSOX1 suppresses tumor growth in vivo. Mol. Cancer. Ther. 2020, 19 (1), 112-122.
34. Zhang, P. Y.; Park, H. J.; Zhang, J.; Junn, E.; Andrews, R. J.; Velagapudi, S. P.; Abegg, D.; Vishnu, K., Costales, M. G.; Childs-Disney, J L., Adibekian, A, Moss, W. N.; Mouradian, M. M.; Disney, M. D., Translation of the intrinsically disordered protein alpha-synuclein is inhibited by a small molecule targeting its structured mRNA. Proc. Natl. Acad. Sci. U.S.A. 2020, 117 (3), 1457-1467.

### Example 2: Materials and Methods

General Methods. All DNA and RNA oligonucleotides were obtained from Integrated DNA Technologies (IDT, Inc.) and Dharmacon, respectively. DNA oligonucleotides were used directly. Fluorescently labeled RNAs were deprotected according to the manufacturer's protocol, desalted on PD-10 Desalting Columns (GE Healthcare Life Sciences) per the manufacturer's instructions, and quantified by UV/Vis spectroscopy on a DU800 UV/Vis spectrometer (Beckman Coulter) by measuring absorbance at 260 nm (90° C.) and the corresponding extinction coefficient.

In Vitro Screening of Functionalized Compounds with Clickable TAMRA Azide. MDA-MB-231 cells were grown in 100 mm dishes. Upon reaching confluency, total RNA was harvested using a Quick-RNA Miniprep Kit (Zymo; R1054) per manufacturer's protocol. Total RNA (2 μg) was folded by heating to 95° C. in 1× Folding Buffer (8 mM Na₂HPO₄, pH 7.0, 185 mM NaCl, and 1 mM EDTA) for 2 min followed by cooling on ice. This solution was then mixed with the compound of interest at a final concentration of 100 μM, and the sample was incubated at room temperature for 30 min. After incubation, samples were irradiated with UV light (365 nm) for 15 min and then added to a “click reaction mix”, composed of TAMRA azide (1 μL, 10 mM; Cat #AZ109, Click Chemistry Tools), CuSO₄ (1 μL, 10 mM), THPTA (1 μL, 50 mM; Cat #1010, Click Chemistry Tools) and sodium ascorbate (1 μL, 250 mM). The samples were incubated at 37° C. for 3 h, and the RNA was purified by ethanol precipitation. The precipitated RNA was dissolved in Nanopure water, and 2 μg RNA was loaded on a 1% (w/v) agarose gel. The agarose gel was then imaged using a Typhoon FLA 9500 variable mode imager (GE Healthcare Life Sciences). Following imaging of TAMRA fluorescence, the agarose gel was stained with SYBR green and imaged to visualize total RNA.

In Vitro Chemical-Crosslinking and Isolation by Pull-down (Chem-CLIP) with Fluorescently Labeled RNA. Cy5-labeled RNA (100 μL, 2 μM) was folded by heating at 95° C. for 2 min in 1× Folding Buffer and cooled on ice. The RNA was then mixed with F1 at the indicated concentrations. The sample was irradiated with UV light (365 nm) for 15 min and then added to 100 μL of azide-disulfide agarose beads (Click Chemistry Tools, 1238-2) pre-washed with 200 μL of 25 mM HEPES, pH 7.0. A click reaction solution was prepared by mixing 30 μL of 250 mM sodium ascorbate, 30 μL of 10 mM CuSO₄, and 30 μL of 50 mM THPTA, which was added to the sample. The sample was incubated while rotating at 37° C. for 2 h, briefly centrifuged, and then the supernatant was decanted. The beads were washed six times with 1× Washing Buffer (10 mM Tris-HCl, pH 7, 4 M NaCl, 1 mM EDTA, and 0.05% (v/v) Tween-20) following by incubation with 1× Releasing Buffer (50 mM TCEP and 100 mM K₂CO₃) at 37° C. for 30 min. An equivalent of iodoacetamide (200 mM) was added to the sample, which was incubated at 37° C. for an additional 30 min. The sample was briefly centrifuged and supernatant containing RNA was carefully transferred to a clean tube. Cy-5 fluorescence in the supernatant was measured with a Molecular Devices SpectraMax M5 plate reader with an excitation wavelength of 640 nm and an emission wavelength of 680 nm. For competitive Chem-CLIP, F1-RIBOTAC was added to the folded the RNA and incubated at room temperature for 15 min prior to the addition of F1. Pull-down was then completed as described above.

In Vitro Binding by Measuring Changes in Fluorescence Polarization. Cy5-labeled RNA was folded by heating at 95° C. for 2 min in 1× Folding Buffer and cooled on ice. Serial dilutions of F1 (200 to 0.4 μM, 1:1 dilutions) were prepared in 1× Folding Buffer with 4% (w/v) bovine serum albumin (BSA) with constant DMSO concentration (2% v/v). The compound solutions (10 μL) were added to a well of non-binding 384-well plate (Corning). An equal volume of folded RNA was adding to each well with a final concentration of 50 nM. The samples were incubated at room temperature for 30 min, and then fluorescence polarization was measured with a Molecular Devices SpectraMax M5 plate reader with an excitation wavelength of 640 nm and an emission wavelength of 680 nm.

Optical Melting Experiments. The thermal stability of WT and mutant model RNAs in the presence and absence of F1-Amide was measured by optical melting. The RNA (1 mM) was prepared in 1× Melting Buffer (8 mM Na₂HPO₄, pH 7, 10 mM NaCl, and 1 mM EDTA) and heated to 95° C. followed by cooling on ice. F1-Amide was added to the final concentration of 2 mM; an equal volume of DMSO was added to the vehicle sample. The absorbance of the solution at 260 nm was measured by a Beckman Coulter DU800 spectrophotometer as a function of temperature, from 12° C. to 85° C. at a rate of 1° C./min. The background absorbance of buffer with DMSO or compound was subtracted before fitting the curve with MeltWin (v3.5)⁷ to determine the melting temperature (Tₘ) and the Gibbs free energy (ΔG_{37° C.}).

Cell Culture. Cellular experiments were carried out in MDA-MB-231 cells (ATCC, HTB-26), cultured at 37° C. with 5% CO₂ in 1′ DMEM (Dulbecco's Modified Eagles Medium; Sigma Aldrich) supplemented with 10% (v/v) fetal bovine serum (FBS; Sigma Aldrich), 2 mM L-alanyl-L-glutamine (Glutagro; Corning), I′ Penicillin/Streptomycin (Corning). Cells were used at a maximum passage number of 20 and checked for mycoplasma contamination (PromoKine, PK-CA91-1024) before performing experiments. Unless stated otherwise, compound treatment was performed by replacing the growth medium with fresh medium that includes compounds at the treatment concentration with 0.1% DMSO(v/v). Antisense oligonucleotides (ASO) were transfected by using RNAiMax (Thermo Fisher) per manufacturer's protocol.

### Construction of RNase L Kock Out (KO) MDA-MB-231 Cells

The CRISPR-edited MDA-MB-231 cell lines used in this study were the same as those previously reported and characterized.⁸ Briefly, lentiviral constructs containing Cas9 and gRNA targeting RNase L mRNA (or a scramble control) were purchased (Transomic Tech) and transfected in HEK293T cells (ATCC CRL-11268) to harvest virus. Transduction to MDA-MB-231 cells were performed in the presence of 6 mg/mL polybrene (Millipore, Cat #TR-1003-G) followed by selection with puromycin.

Cross-linking to RNA, DNA, and Proteins in Live Cells with Clickable TAMRA Azide. MDA-MB-231 cells were seeded in 100 mm dishes and allowed to reach ˜80% confluency. The cells were then treated with 20 mM of compound in growth medium and incubated for 16 h. Cells were washed with 1×DPBS and irradiated with UV light for 10 min. Total RNA was harvested using a Zymo Quick-RNA Miniprep Kit per the manufacturer's protocol with DNase and proteinase treatment. Total DNA was harvested using a Zymo Quick-DNA Miniprep Kit per the manufacturer's protocol with RNase and proteinase treatment. Total protein was harvested by using Mammalian Protein Extraction Reagent (M-PER, Thermo Scientific) per the manufacturer's protocol with RNase and DNase treatment. Each sample (4 μg for RNA or DNA, 25 μg for proteins) was then incubated in a “click reaction mixture” with TAMRA azide as described in In Vitro Screening of Functionalized Compounds with Clickable TAMRA Azide. DNA and RNA samples were separated by using a 1.0% (w/v) and 1.5% (w/v) agarose gel in TBE buffer, respectively. Protein samples were loaded to 10% SDS-polyacrylamide gel. All samples were first imaged with TAMRA channel by using a Typhoon FLA 9500 variable mode imager (GE Healthcare Life Sciences). RNA and DNA were then visualized by SYBR green staining, and total proteins were visualized by a Coomassie Brilliant Blue staining (Bio-Rad) per manufacturer's protocols.

Chemical-Cross-linking and Isolation by Pull-down (Chem-CLIP) in Live Cells. MDA-MB-231 cells were seeded in 60 mm dishes and allowed to reach ˜80% confluency. The cells were then treated with 20 μM of compound in growth medium and incubated for 16 h. Cells were washed with 1×DPBS and irradiated with UV light for 10 min. Total RNA was harvested using a Zymo Quick-RNA Miniprep Kit per the manufacturer's protocol with DNase treatment. Total RNA was chemically fragmented by using an NEBNext Magnesium RNA Fragmentation Module (E6150S) per manufacturer's protocol to achieve final lengths between 100-150 nucleotides Pull-down of cross-linked RNAs was performed by adding 10 μg of total RNA to 100 μL of azide-disulfide agarose beads (Click Chemistry Tools, 1238-2) pre-washed with 25 mM HEPES, pH 7.0. The click reaction and pull-down were completed as described in “In Vitro Chemical-Crosslinking and Isolation by Pull-down (Chem-CLIP) with Fluorescently Labeled RNA”. The pulled down RNA was purified by RNA CleanXP beads (Beckman, A66514) per manufacturer's protocol.

For dose response studies, cells were treated with varying concentration of F1 (1, 5, 20 μM) as described above for 16 h. For time course studies, cells were treated with 20 μM of for 1, 4, 8, or 16 h. With the exception of compound concentration or time at which total RNA was harvested, the remaining steps of the protocol were completed as described above for both concentration- and time-dependent studies.

RNA-seq Library Preparation and Data Analysis. The quality of the pulled down RNA was analyzed by using an Agilent 2100 Bioanalyzer RNA nanochip as well as to confirm fragment lengths. RNA concentration was quantified by Qubit 2.0 Fluorometer (Invitrogen). The input RNA (200 ng) was depleted of ribosomal RNA with NEBNext rRNA Depletion Module (E6310) according to manufacturer's recommendations. Library preparation was performed with NEBNext Ultra II Directional RNA kit (E7760) per manufacturer's protocols. Briefly, the rRNA-depleted samples were reverse transcribed with random hexamer primers to generate first strand cDNA, followed by second strand synthesis with dUTP instead of dTTP. The cDNA was end repaired and adenylated at their 3′ ends, followed by adaptor ligation. The strand information of the RNA was preserved by using USER enzyme (Uracil-specific excision reagent) to degrade the second strand. The cDNA was then PCR amplified with barcoded Illumina-compatible primers to generate the final libraries. These libraries were loaded into a NextSeq 500 v2.5 flow cell and sequenced with 2×40 bp paired-end chemistry. All fastq files were aligned to the human genome by STAR.⁹ The output bam files were processed by Genrich (v0.6.1, available at https://github.com/jsh58/Genrich) for peak calling to identify regions of enrichment (p<0.001 and False Discovery Rate=1%). Fold enrichment was calculated by Deeptool¹⁰ and visualized by IGV browser.¹¹ A minimum read count of 5 and a minimum fold enrichment of 1.5 were applied filter to filter out low-confidence peaks.

Proteomic Sample Preparation and Analysis. MDA-MB-231 cells were seeded in 60 mm dishes and allowed to reach ˜80% confluency. The cells were then treated with 20 mM of compound in growth medium and incubated for 16 h. Cells were washed with 1×DPBS and irradiated with UV light for 10 min. For each condition, two biological replicates were prepared, and each biological replicate was split into three technical replicates. Cells were then homogenized by sonication and resuspended in 1×DPBS (0.35 mL), followed by incubation with biotin azide (50 mM), tris(2-carboxyethyl) phosphine hydrochloride (TCEP) (1 mM), tris[(1-benzyl-1H-1,2,3-triazol-4-yl)methyl]amine (TBTA) (100 mM), and copper (II) sulfate (1 mM) at room temperature for 1 h. Proteins were isolated by adding 1.4 mL of MeOH, 0.35 mL of chloroform, and 1.05 mL of water, followed by centrifugation at 14,000×g at 4° C. for 5 min. Total proteins were resuspended in 2% (w/v) SDS in 1×PBS via sonication, followed by centrifugation at 4,700×g at 4° C. for 5 min. The supernatant was transferred to a new tube and diluted with 1×PBS to afford a final SDS concentration as 0.2% (w/v). Streptavidin agarose beads (ProteoChem) were added for pull-down of cross-linked proteins, and the sample was incubated at room temperature for 4 h, followed by washing with 1% SDS in 1×PBS (1×10 mL), 1×PBS (3×10 mL), and finally water (3×10 mL).

Pulled-down proteins were released from beads by resuspending in 6 M urea/1×PBS (0.5 mL) supplemented with 10 mM of TCEP at room temperature for 30 min. Todoacetamide (25 mM final concentration) was added to the mixture, followed by incubation at room temperature for another 30 min in the dark. The beads were then diluted with 0.7 mL of 1×PBS, pelleted by centrifugation (1,400×g, 2 mins), and resuspended in 150 ml of digestion solution (2 M urea, 1 mM CaCl₂), 3 mg/mL trypsin, pH=8) overnight at 37° C. To the samples was added 1 volume of isopropanol containing 1% (v/v) trifluoroacetic acid, followed by a styrenedivinylbenzene reverse-phase sulfonate (SDB-RPS) StageTip 12 Peptides were resuspended in 0.1% (v/v) formic acid (FA) in water and analyzed using Proxeon EASY-nLC 1200 nano-UHPLC coupled to QExactive HF-X Quadrupole-Orbitrap mass spectrometer (Thermo Scientific). The chromatography column consisted of a 40 cm long, 75 μm microcapillary capped by a 5 μm tip and packed with ReproSil-Pur 120 C18-AQ 2.4 μm beads (Dr. Maisch GmbH). Peptides were eluted into the mass spectrometer at a flow rate of 300 nL/min with 0.1% FA in H2O (Buffer A) and 0.1% FA in MeCN (Buffer B) over a 90-minute linear gradient (5-35% Buffer B) at 50° C. Data were acquired in data-dependent mode (top-20, NCE 28, R=15,000) after full MS scan (R=60,000, m/z 400-1,300). Dynamic exclusion was set to 30 s and isotope exclusion was enabled.

The MS data were analyzed with MaxQuant13 (V1.6.1.0) and searched against the human proteome (Uniprot) and a common list of contaminants included in MaxQuant. The first peptide search tolerance was set at 20 ppm, and 10 ppm was used for the main peptide search. The fragment mass tolerance was set to 0.02 Da. The false discovery rate for peptides, proteins, and sites identification was set to 1%. The minimum peptide length was set to six amino acids and peptide re-quantification was enabled with the minimal number of peptides per protein set to 2. Methionine oxidation and protein N-terminal acetylation were searched as variable modifications, and carbamidomethylation of cysteines was searched as a fixed modification.

Analysis of mRNA Abundance by RT-qPCR. Unless stated otherwise, all RT-qPCR experiments were performed to measure nascent RNA levels by using Click-it Nascent RNA Capture Kit (Cat #C₁₀₃₆₅, Thermo Fisher) per the manufacturer's protocols. Briefly, cells were treated with 0.2 mM of ethynyl uridine (EU) in growth medium for 16 h, after which the medium was replaced with fresh growth medium lacking EU. Compound or ASO treatment was performed at the same time of initial EU treatment, and compounds were replenished after replacing the medium to remove EU. Total RNA was extracted by using a Zymo Quick-RNA Miniprep Kit as per manufacturer's protocol with DNase treatment. The click reaction and pull-down of nascent RNA were performed as described in “In Vitro Chemical-Crosslinking and Isolation by Pull-down (Chem-CLIP) with Fluorescently Labeled RNA”. Reverse transcription (RT) was performed by using 300 ng total RNA and a QScript cDNA synthesis kit (QuantaBio) per manufacturer's protocols qPCR was performed in 384-well plates by using Power SYBR Green Master Mix (Life Technologies) on a QuantStudioS™ Cycler (Applied Biosystems) using the “Comparative Ct with Melt” method.

Measuring Protein Abundance by Western Blotting. MDA-MB-231 cells were seeded in 6-well plates at ˜50% confluency and treated with vehicle (DMSO) or compound as described above but without EU treatment. After 16 h, the compound-containing growth medium was removed and replaced with fresh medium containing compound (re-dosing). After an additional 32 h incubation, total protein was harvested (48 h total treatment time). The cells were then washed twice with cold 1×DPBS, and total protein was harvested by using Mammalian Protein Extraction Reagent (M-PER, Thermo Scientific) per the manufacturer's protocol. Protein concentration was quantified by using BCA Protein Assay Kit (Pierce), and the proteins were separated by using 10% SDS-polyacrylamide gel (20 μg protein per lane). After gel electrophoresis in 1× Running Buffer (50 mM MOPS, 50 mM Tris-base, pH 7.5, 1 mM EDTA, and 0.1% (w/v) SDS) at 120 V for 1 h, the proteins were transferred to a PVDF membrane (0.45 μm, Cytiva) at 300 mA for 1 h in 1× Transfer Buffer (25 mM Bicine, 25 mM Tris-base, pH 7.5, 1:4 MeOH:H₂O). The membrane was blocked with 5% (w/v) non-fat dry milk in TBST (1×TBS containing 0.1% (v/v) Tween-20) for 1 h at room temperature. The primary antibody for QSOX1 protein (Cat #10092-932, Proteintech) or SQSTM1 protein (Cat #ab 109012, Abcam) was incubated with 1:1000 dilutions at 4° C. for 16 h. The membrane was then washed by TBST four times (5 min each) and then incubated with the secondary antibody (Cat #7074S, Cell Signaling) with 1:5000 dilution at room temperature for 2 h. The membrane was washed with TBST four times (5 min each) and imaged by using a SuperSignal West Pico Chemiluminescent Substrate (Pierce) per manufacturer's protocol. The blot was stripped by washing the membrane in 1× Stripping Buffer (200 mM glycine, pH 2.2, 4 mM SDS, 1% (v/v) Tween 20) at room temperature for 30 min, followed by blocking again as described above. The antibody for GAPDH protein (Cat #97166, Cell Signaling) was then applied with 1:2000 dilutions followed by the same procedure to image as described above. Expression levels of proteins were quantified based on band intensity by using ImageJ. QSOX1 and SQSTM1 signals were normalized to GAPDH signal for each sample.

Invasion Assay. MDA-MB-231 cells were seeded in 6 mm dishes and treated with compound or transfected by ASO for 48 h as described in Cell Culture. After treatment for 48 h, the medium was replaced with fresh medium that contains same concentration of compound but lacking FBS for 8 h (serum starvation). ThinCert (GBO) 24-well inserts with 8 μm pores were coated with 100 μL of 0.5 mg/mL Matrigel (Fisher Scientific: CB40234) per well, and then cells were added into the insert (50,000 cells per well). Fresh growth medium with FBS was added to a 24-well plate (600 μL each well), and the inserts containing cells were placed on the top, allowing medium to cover the bottom of the insert. After 16 h, all medium was removed, and cells were washed by 1×DPBS twice. Cells were then fixed with 3% (w/v) paraformaldehyde (PFA) in 1×DPBS at room temperature for 30 min, followed by staining with Crystal Violet (10 mg/mL; 4:1H₂O:MeOH) at room temperature for 20 min. A cotton swab was used to gently remove non-invasive cells on sitting on top of the Matrigel. The inserts were then imaged under a regular optical microscope (3 views per well), and the number of invasive cells were counted manually.

Proliferation Assay. MDA-MB-231 cells were seeded in 96-well plates (Corning) at ˜40% confluency and treated with compound or transfected with ASO as described in Cell Culture. Compound was replenished every 16 h by replacing all the growth medium with freshly prepared medium containing same concentration of compounds. After 48 h total treatment period, proliferation was measured by using CellTiter 96 AQueous One Solution Cell Proliferation Assay (Cat #G3582, Promega) per manufacturer's protocol.

TABLE 1
Sequences of oligonucleotides used in these studies.

| Oligonucleotide | Sequence 5′ ® 3′ | Experiment | Supplier |
|---|---|---|---|
| QSOX1-F | GTG CTT GCG TGT GGT GGT GAG C | qPCR | IDT |
| QSOX1-R | CAG CGG GTC GGA AGG CGA ATA | qPCR | IDT |
| | GAG | | |
| SQSTM1-F | TAT GGC GTC GCT CAC CGT GAA GG | qPCR | IDT |
| SQSTM1-R | CAG CCT CGG CTT CCG CCT CAG | qPCR | IDT |
| 18S-F | GTA ACC CGT TGA ACC CCA TT | qPCR | IDT |
| 18S-R | CCA TCC AAT CGG TAG TAG CG | qPCR | IDT |
| QSOX1-ASO^{a} | UGU TGG TCT CCT CAG C | Cellular assays | IDT |
| Scrambled-ASO^{a} | GUG AGG GUC A | Cellular assays | IDT |
| Cy5-QSOX1- | Cy-5 GGU GCU UGC GUG UGG UGG | In vitro assays | Dharmacon |
| WT^{b} | UGA GCG CAG CGC C | | |
| Cy5-QSOX1- | Cy-5 GGU GCU UGC GUG UGG UGG | In vitro assays | Dharmacon |
| BP^{b} | UGA GCG CAA GCG CC | | |

^{a}Linkages of all nucleotides are phosphorothioate.
^{b}2′-O-Methoxyelthyl (MOE) nucleotides are indicated in bold.

### Example 3: Synthetic Methods and Characterization

Abbreviations: DCM: Dichloromethane; DIPEA: Diisopropyl ethyl amine; DMF: N, N-Dimethylformamide; DMSO: Dimethyl sulfoxide; EtOAc: Ethyl acetate; HATU: Hexafluorophosphate azabenzotriazole tetramethyl uranium; HOAt, 3-hydroxytriazolo[4,5-b]pyridine; HPLC: High-performance liquid chromatography; MeOH: Methanol; TFA: Trifluoro acetic acid; POCl₃: Phosphoryl chloride.

General Synthetic Methods. Amine derivatives were purchased from Enamine. 3-(3-(but-3-yn-1-yl)-3H-diazirin-3-yl) propanoic acid was purchased from Sigma Aldrich. N, N-dimethylformamide (DMF, anhydrous) was purchased from EMD and used without further purification. Claricep S-series pre-packed silica columns were purchased from Agela-Technologies. Flash chromatography was carried out on the Biotage Isolera One automated flash chromatography system with pre-packed Claricep S-series (40-60 μm) normal-phase flash columns of various sizes. ¹H NMR spectra were collected on a Bruker 400 MHz NMR spectrometer; ¹³C NMR spectra were collected on either Bruker 400 MHz NMR spectrometer or Bruker 600 MHz NMR spectrometer.

Preparative HPLC was performed using a Waters 1525 Binary HPLC pump equipped with a Waters 2487 dual absorbance detector system and a Waters Sunfire C18 OBD 5 μm, 19×150 mm S-14 column. Absorbance was monitored at 254 and 345 nm. A linear gradient with a flowrate of 5 mL/min from 0-100% methanol in water with 0.1% (v/v) TFA over 100 min was used for small molecule purification. Purity was assessed by analytical HPLC using a Waters Symmetry C_{18 5} μm, 4.6×150 mm column with a flow rate of 1 mL/min and a linear gradient from 0-100% methanol in water with 0.1% (v/v) TFA over 60 min. Absorbance was monitored at 254 and 345 nm. Mass spectra were recorded on an Applied Biosystems 4800 plus MALDI-TOF/TOF analyzer using an α-cyano-4-hydroxycinnamic acid matrix.

General Procedure for Diazirine Probe Synthesis and Purification: The general synthesis scheme of diazirine probes was adapted from the literature.⁵ The following were added to a vial containing 1 mL DMF: 3-(3-(but-3-yn-1-yl)-3H-diazirin-3-yl) propanoic acid (1.5 equiv.), HATU (2 equiv.), HOAt (2 equiv.) and DIPEA (3 equiv.), and the reaction was stirred for 10 min at room temperature. After 10 min, an amine (1.0 equiv.) was added, and the sample was stirred at temperature for 12 h. Compounds were purified by flash chromatography or by HPLC as described in General Synthetic Methods.

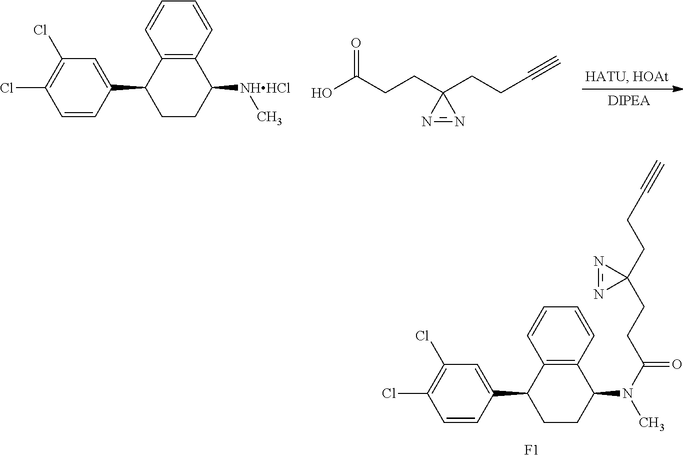

Scheme 1: Compound F1 was synthesized as described in General Procedure for Diazirine Probe Synthesis and Purification. The starting amine was purchased from Enamine (EN300-122632) and validated by 1H NMR (600 MHz, DMSO d6) δ (ppm) 9.62 (d, J=17.5 Hz, 1H), 7.73 (d, J=7.4 Hz, 1H), 7.67 (d, J=1.9 Hz, 1H), 7.59 (d, J=8.3 Hz, 1H), 7.38 (dd, J=8.3, 1.9 Hz, 1H), 7.34-7.23 (m, 1H), 6.76 (d, J=7.5 Hz, 1H), 4.42 (s, 1H), 4.16 (dd, J=9.8, 5.6 Hz, 1H), 2.64 (s, 2H), 2.33-2.24 (m, 1H), 2.22-2.13 (m, 1H), 2.09-1.94 (m, 1H). 13C NMR (600 MHz, DMSO d6) δ (ppm) 146.19, 139.37, 130.51, 130.43, 130.38, 129.93, 129.79, 129.18, 129.13, 128.55, 128.47, 125.93, 54.36, 43.55, 29.70, 25.93, 22.24. The optical rotation was measured to be +45.2° at 21° C., 589 nm with 1 g/100 mL EtOH. All characterizations fully match with the reported results from the supplier's certificate of analysis.

Characterization of F1. F1 was obtained in 78% yield (15.5 mg, 34.1 μmol). ¹H NMR, CDCl₃ (400 MHZ) δ (ppm): 1.68-1.73 (t, 4H, CH₂), 1.95-2.0 (m, 3H, CH, CH₂), 2.05-2.08 (m, 3H, CH₂), 2.11-2.24 (m, 3H, CH₂), 2.70 (s, 3H, CH₃), 4.20-4.24 (m, 1H, CH), 5.91-5.94 (m, 1H, CH), 6.77-6.85 (m, 1H, ArH), 6.96-7.01 (m, 1H. ArH), 7.08-7.14 (m, 2H, ArH), 7.17-7.20 (t, 1H, ArH), 7.22-7.27 (m, 1H, ArH), 7.33-7.34 (m, 1H, ArH), ¹³C NMR, CDCl₃ (600 MHz) δ (ppm)=13.39, 21.53, 22.66, 27.64, 27.81, 28.09, 30.17, 30.90, 32.74, 42.76, 43.01, 52.72, 69.34, 82.82, 126.64, 127.23, 127.41, 127.54, 128.01, 130.11, 130.61, 130.87, 131.15, 132.30, 135.98, 138.29, 147.03, 171.95. HR-MS: Calculated for [C₂₅H₂₆Cl₂N₃O]⁺, 454.1453; found 454.1450.

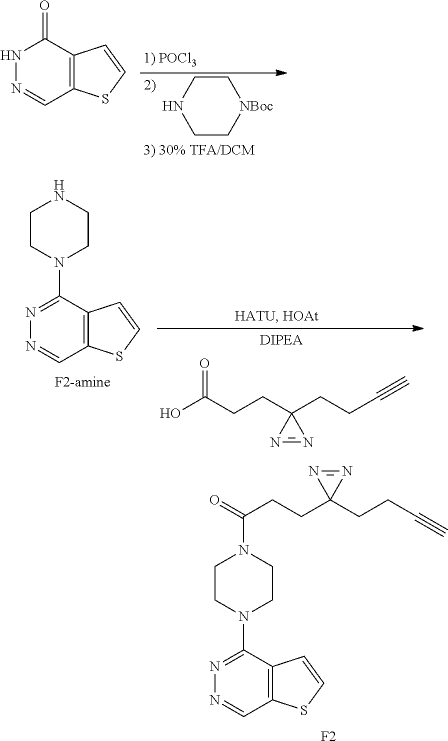

Scheme 2: Scheme for the synthesis of F2.

Synthesis of F2. Thieno[2,3-d]pyridazin-4 (5H)-one 11 (30 mg, 197 μmol) was refluxed in POCl₃ (1 mL) for 12 h. The solvent was evaporated and carefully quenched by slowly adding it to warm water with constant stirring. The compound was then dried under vacuum for 12 h and resuspended in isopropanol (1 mL), to which tert-butyl piperazine-1-carboxylate was added. The reaction was heated at 120° C. for 2 h and purified by column chromatography as described in General Synthetic Methods. The Boc protecting group was then removed by treatment with 30% (v/v) TFA in DCM and stirring the reaction at room temperature for 2 h. The resulting compound, F2-amine, was purified by HPLC as described in General Synthetic Methods. F2 was then synthesized from F2-amine as described in General Procedure for Diazirine Probe Synthesis and Purification. F2 was obtained in 40% yield (6.6 mg, 17.9 μmol). 1H NMR, CDCl₃ (400 MHZ) δ (ppm): 1.68-1.72 (t, 2H, J=7.4 Hz, CH₂), 1.90-1.94 (t, 2H, J=7.5 Hz, CH₂), 2.00-2.01 (t, 1H, J=2.6 Hz, CH), 2.03-2.08 (td, 2H, J=7.4, 2.5 Hz, CH₂), 2.12-2.16 (s, 2H, J=7.5 Hz, CH₂), 3.70-3.73 (m, 2H, CH₂), 3.86-3.92 (m, 6H, CH₂), 7.71-7.73 (d, 1H, J=5.4 Hz, ArH), 8.21-8.23 (d, 1H, J=5.4 Hz, ArH), 9.67 (s, 1H, ArH). ¹³C NMR, CDCl₃ (400 MHZ) δ (ppm)=13.38, 17.46, 18.66, 26.87, 27.94, 32.80, 41.36, 44.84, 45.70, 48.37, 49.71, 53.43, 69.15, 82.75, 122.01, 129.27, 132.92, 140.22, 140.93, 157.45, 169.99. HR-MS: Calculated for [C₁₈H₂₁N₆OS]⁺, 369.1498; found 369.1492.

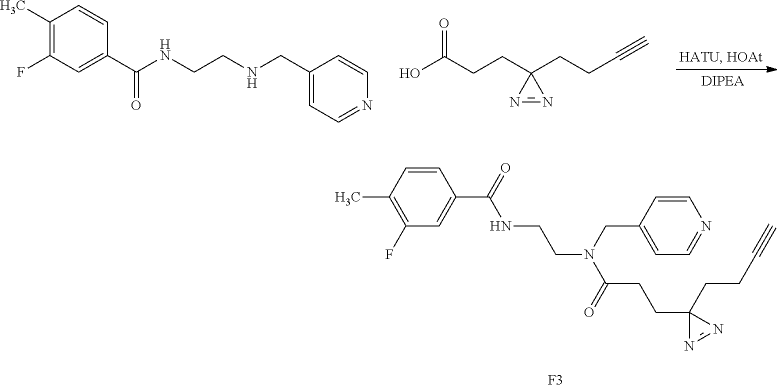 [SMILES: Cc1ccc(C(=O)NCCNCc2ccncc2)cc1F ; C#CCCC1(CCC(=O)O)N=N1 ; C#CCCC1(CCC(=O)N(CCNC(=O)c2ccc(C)c(F)c2)Cc2ccncc2)N=N1 ; "F3"]

Scheme 3: Compound F3 was synthesized as described in General Procedure for Diazirine Probe Synthesis and Purification.

Characterization of F3 was obtained in 44.5% yield (8.1 mg, 18.6 μmol). 1H NMR, MeOD (400 MHZ) δ (ppm): 1.50-1.57 (m, 2H,), 1.73-1.78 (m, 4H), 1.93-1.97 (m, 2H), 2.07-2.11 (m, 2H), 2.25-2.27 (m, 2H), 2.32-2.37 (m, 1H), 3.57-3.62 (m, 4H), 4.76 (m, 2H), 7.34-7.37 (t, 1H), 7.45-7.55 (m, 2H), 7.80-7.89 (d, 2H), 8.71-8.73 (d, 2H). ¹³C NMR, CDCl₂ (600 MHz) δ (ppm)=12.42, 13.08, 26.30, 27.73, 32.21, 37.87, 68.97, 82.12, 113.58, 122.54, 129.15, 131.60, 133.32, 146.11, 148.15, 160.32, 162.08, 167.67, 173.24. HR-MS: Calculated for [C₂₄H₂₇FN₅O₂]⁺, 436.2149; found 436.2151.

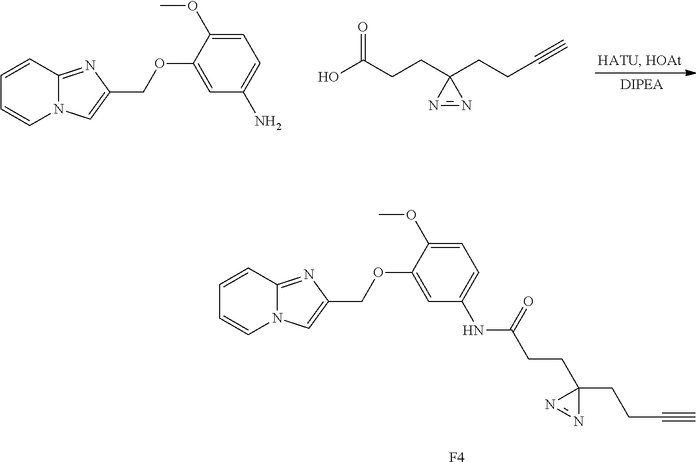

Scheme 4: Compound F4 was synthesized as described in General Procedure for Diazirine Probe Synthesis and Purification.

Characterization of F4 was obtained in 96% yield (17.8 mg, 42.6 μmol). ¹H NMR, CDCl₃ (600 MHz) δ (ppm): 1.63-1.66 (t, 2H, J=7.4 Hz, CH₂), 1.85-1.88 (t, 2H, CH₂), 1.97-2.02 (m, 3H, CH₂, CH), 2.08-2.011 (t, 2H, CH₂), 3.83 (s, 3H, CH₃), 5.29 (s, 2H, CH₂), 6.79-6.80 (d, 1H, J=8.7 Hz, ArH), 6.84-6.86 (t, 1H, J=6.7 Hz, ArH), 7.13 (d, 1H, J=2.2 Hz, ArH), 7.24-7.27 (m, 2H, ArH), 7.60-7.62 (d, 1H, J=9.1 Hz, ArH), 7.69 (s, 1H, ArH), 8.10-8.11 (d, 2H, J=6.9 Hz, ArH), ¹³C NMR, CDCl₃ (600 MHz) δ (ppm): 13.41, 27.96, 28.29, 31.02, 32.34, 41.04, 56.21, 64.86, 69.22, 82.83, 106.74, 111.43, 111.88, 113.08, 113.27, 116.71, 126.12, 131.59, 141.44, 144.20, 146.22, 147.50, 169.56. HR-MS: Calculated for [C₂₃H₂₄N₅O₃]⁺, 418.1879; found 418.1872.

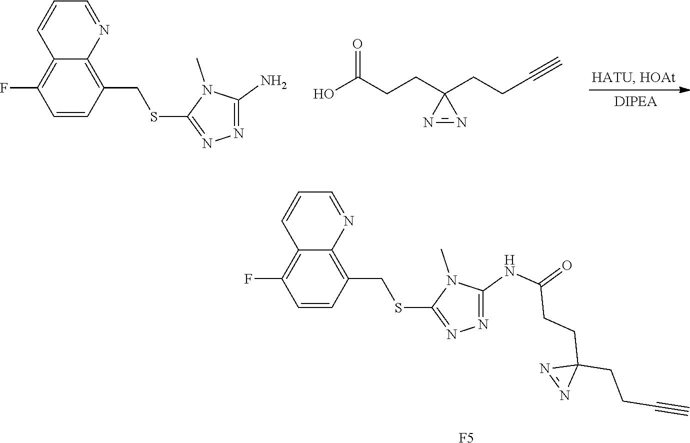 [SMILES: Cn1c(N)nnc1SCc1ccc(F)c2cccnc12 ; C#CCCC1(CCC(=O)O)N=N1 ; C#CCCC1(CCC(=O)Nc2nnc(SCc3ccc(F)c4cccnc34)n2C)N=N1 ; "F5"]

Scheme F5: Compound F5 was synthesized as described in General Procedure for Diazirine Probe Synthesis and Purification.

Characterization of F5 was obtained in 49% yield (9.6 mg, 22 μmol). ¹H NMR, CDCl₃ (600 MHz) δ: 1.66-1.69 (t, 2H, J=7.5 Hz, CH₂), 1.88-1.90 (t, 2H, J=7.4 Hz, CH₂), 1.97-1.98 (t, 1H, J=2.6 Hz, CH), 2.01-2.04 (td, 2H, J=7.5, 2.6 Hz, CH₂), 2.33-2.36 (t, 2H, J=7.4 Hz, CH₂), 3.33 (s, 3H, CH₃), 4.95 (s, 2H, CH₂), 7.14-7.17 (dd, 1H, J=9.4, 8.0 Hz, ArH), 7.48-7.50 (dd, 1H, J=8.4, 4.2 Hz, ArH), 7.68-7.70 (dd, 1H, J=7.9, 6.0 Hz, ArH), 8.43-8.44 (dd, 1H, J=8.4, 1.7 Hz, ArH), 8.96-8.97 (dd, 1H, J=4.2, 1.7 Hz, ArH). ¹³C NMR, CDCl₃ (600 MHz) δ (ppm): 13.26, 27.9, 30.74, 31.16, 32.24, 33.07, 40.94, 69.25, 82.71, 109.90, 119.32, 121.47, 129.72, 130.99, 146.50, 150.22, 150.61, 156.85, 158.55. HR-MS: Calculated for [C₂₁H₂₁FN₇OS]⁺, 438.1512; found 438.1509.

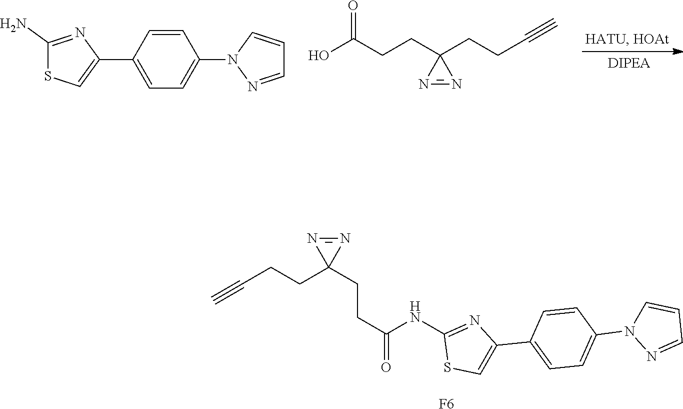

Scheme 6: Compound F6 was synthesized as described in General Procedure for Diazirine Probe Synthesis and Purification.

Characterization of F6 was obtained in 39% yield (6.9 mg, 17.7 μmol). ¹H NMR, CDCl₃ (400 MHz) δ (ppm): 1.70-1.74 (t, 2H, CH₂), 2.01-2.03 (m, 2H, CH₂), 2.05-2.09 (m, 3H, CH, CH₂), 2.35-2.39 (t, 2H, CH₂), 7.19 (s, 1H, ArH), 7.38 (s, 1H, ArH), 7.46-7.48 (d, 2H, ArH), 7.93-7.95 (d, 2H, ArH), 8.44 (s, 1H, ArH). ¹³C NMR (600 MHz, DMSO-d6) δ (ppm): 174.86, 171.03, 158.43, 150.69, 139.60, 135.02, 128.55, 127.44, 122.26, 121.00, 109.29, 82.40, 69.11, 54.52, 42.52, 32.04, 27.75, 17.43, 15.99, 11.89. HR-MS: Calculated for [C₂₀H₁₉N₆OS]³⁰ , 391.1341; found 391.1337.

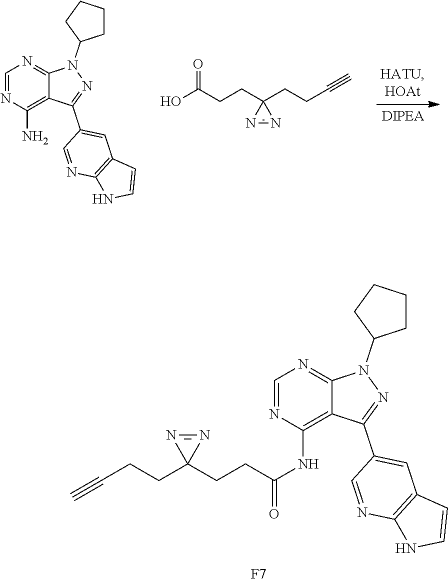 [SMILES: Nc1ncnc2c1c(-c1cnc3[nH]ccc3c1)nn2C1CCCC1 ; C#CCCC1(CCC(=O)O)N=N1 ; C#CCCC1(CCC(=O)Nc2ncnc3c2c(-c2cnc4[nH]ccc4c2)nn3C2CCCC2)N=N1 ; "F7"]

Scheme 7: Compound F7 was synthesized as described in General Procedure for Diazirine Probe Synthesis and Purification.

Characterization of F7. F7 was obtained in 53% yield (3.7 mg, 7.9 μmol). 1H NMR (400 MHz, CD3OD): δ=8.74 (s, 1H), 8.65 (s, 1H), 8.58 (s, 1H), 7.64 (d, J=4 Hz, 1H), 6.79 (d, J=4 Hz, 1H), 5.48 (quint, J=7 Hz, 1H), 2.30-2.15 (m, 5H), 2.12 (m, 2H), 2.08-1.97 (m, 2H), 1.88 (td, J=8-2 Hz, 2H), 1.85-1.78 (m, 2H), 1.40 (t, J=7 Hz, 2H), 1.30 (t, J=8 Hz, 2H). ¹³C NMR (600 MHz, DMSO-d6): δ=173.36, 158.13, 152.84, 148.56, 148.09, 142.33, 128.1, 127.79, 127.43, 127.07, 119.76, 100.70, 100.31, 83, 29, 71.85, 71.81, 32.1, 31.95, 31.92, 31.47, 27.98, 27.45, 24.37, 24.34, 12.68. HR-MS: Calculated for [C₂₅H₂₆N₉O]⁺, 468.2262; found 468.2259.

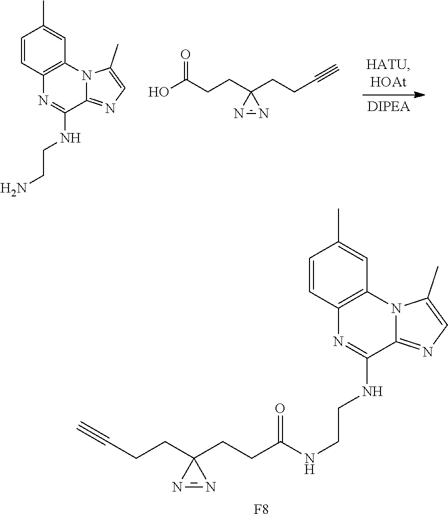

Scheme 8: Compound F8 was synthesized as described in General Procedure for Diazirine Probe Synthesis and Purification.

Characterization of F8. F8 was obtained in 95% yield (8.0 mg, 19.8 μmol). 1H NMR (400 MHZ, DMSO-d6): δ=8.00 (s, 1H), 7.59 (d, J=8 Hz, 1H), 7.30-7.22 (m, 2H), 3.77 (m, 2H), 3.52 (m, 2H), 2.88 (s, 3H), 2.48 (s, 3H), 2.21 (t, J=3 Hz, 1H), 1.98 (m, 2H), 1.91 (td, J=8-3 Hz, 2H), 1.66 (m, 2H), 1.50 (t, J=8 Hz, 2H); ¹³C NMR (125 MHz, DMSO-d6). ¹³C NMR (600 MHz, DMSO-d6): δ=171.45, 158.04, 146.20, 132.50, 132.47, 131.79, 131.72, 129.77, 127.49, 125.54, 115.74, 83.28, 71.83, 53.68, 40.04, 31.44, 29.55, 28.29, 28.12, 21.09, 12.69, 8.72. HR-MS: Calculated for [C₂₂H₂₆N₇O]⁺, 404.2199; found 404.2194.

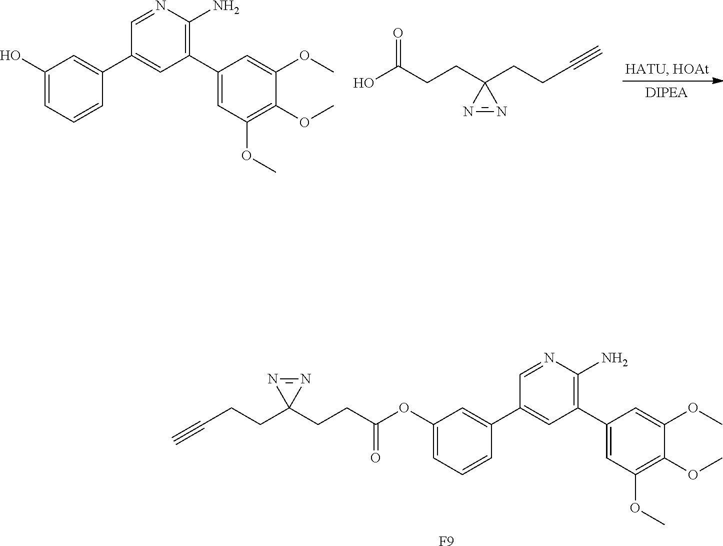

Scheme 9: F9 was synthesized as described in General Procedure for Diazirine Probe Synthesis and Purification.

Characterization of F9. F9 was obtained in 52% yield (2.6 mg, 5.2 μmol). 1H NMR (600 MHZ, CD3OD): δ=8.21 (br, 1H), 7.71 (m, 1H), 7.46 (m, 2H), 7.36 (m, 1H), 7.07 (m, 1H), 6.79 (s, 2H), 3.89 (s, 6H), 3.81 (s, 3H), 2.47 (t, J=7, 2H), 2.27 (t, J=3 Hz, 1H), 2.06 (td, J=7-3 Hz, 2H), 1.91 (t, J=7 Hz, 2H), 1.68 (t, J=7 Hz, 2H). ¹³C NMR (600 MHz, DMSO-d6): δ=170.77, 166.98, 153.28, 131.09, 137.59, 131.77, 131.68, 130.24, 128.78, 124.14, 123.51, 122.11, 120.92, 119.29, 106.29, 83.28, 71.91, 60.13, 56.01, 53.66, 31.30, 29.74, 28.42, 27.47, 13.97. HR-MS: Calculated for [C₂₈H₂₉N₄O₅]⁺, 501.2138; found 501.2135.

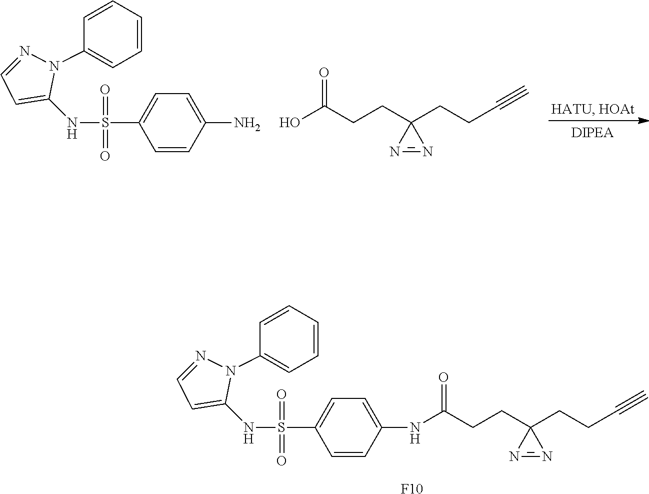

Scheme 10: Compound F10 was synthesized as described in General Procedure for Diazirine Probe Synthesis and Purification.

Characterization of F10. F10 was obtained in 95% yield (3.6 mg, 7.8 μmol). 1H NMR (400 MHz, CD3OD): δ=7.63-7.59 (m, 2H), 7.54-7.49 (m, 3H), 7.40-7.34 (m, 3H), 7.23-7.19 (m, 2H), 6.06 (d, J=4 Hz, 1H), 2.23-2.18 (m, 2H), 1.84 (t, J=8 Hz, 2H), 1.71 (t. J=8 Hz, 1H), 1.66-1.54 (m, 4H). ¹³C NMR (125 MHz, DMSO-d6): δ=173.45, 170.72, 143.33, 139.72, 138.34, 128.89, 128.16, 127.64, 124.51, 118.87, 112.62, 83.39, 72.00, 31.59, 31.48, 28.32, 27.99, 12.76. HR-MS: Calculated for [C₂₃H₂₃N₆O₃S]⁺, 463.1552; found 463.1548.

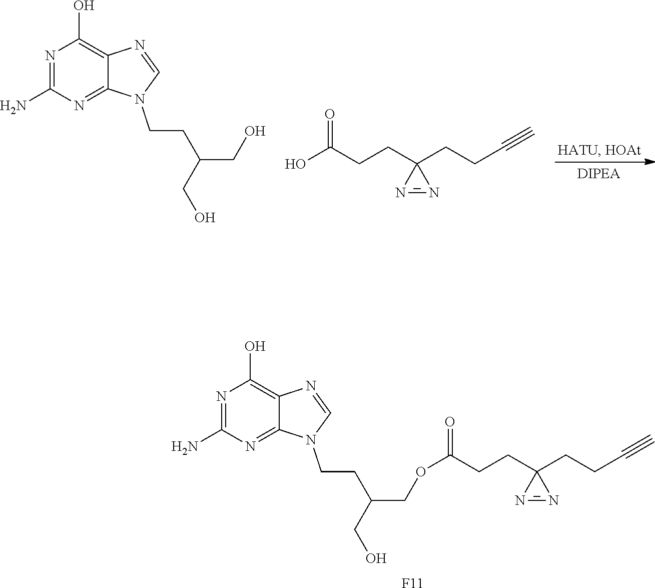

Scheme 11: Compound F11 was synthesized as described in General Procedure for Diazirine Probe Synthesis and Purification.

Characterization of F11. F11 was obtained in 95% yield (2.8 mg, 7.0 μmol). 1H NMR (400 MHz, CD3OD): δ=7.65-7.60 (m, 1H), 4.38-4.14 (m, 4H), 3.67-3.55 (m, 2H), 2.27 (t, J=4 Hz, 1H), 2.22-2.11 (m, 2H), 2.03 (m, 1H), 1.99 (m, 1H), 1.78 (td, J=4-8 Hz, 2H), 1.61 (t, J=8 Hz, 2H), 1.41-1.37 (m, 3H). ¹³C NMR (600 MHz, DMSO-d6): δ=171.91, 156.98, 153.43, 151.24, 137.54, 116.75, 83, 71.78, 64.29, 63.53, 37.53, 31.32, 29.07, 27.91, 27.40, 22.16, 18.94, 12.66. HR-MS: Calculated for [C₁₈H₂₄N₇O₄]⁺, 402.1890; found 402.1885.

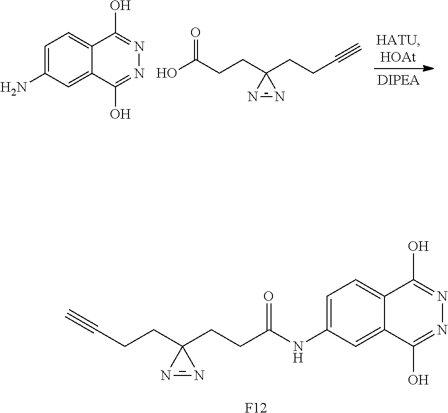

Scheme 12: Compound F12 was synthesized as described in General Procedure for Diazirine Probe Synthesis and Purification.

Characterization of F12. F12 was obtained in 25% yield (1.2 mg, 3.7 μmol). 1H NMR (400 MHz, CD3OD): δ=8.45 (d, J=4 Hz, 1H), 8.14 (d, J=8 Hz, 1H), 8.03 (dd, J=4-8 Hz, 1H), 2.31-2.26 (m, 3H), 2.06 (td, J=1.8-8 Hz, 2H), 1.89 (t, J=8 Hz, 2H), 1.67 (t, J=8 Hz, 2H), 13C NMR (600 MHZ, DMSO-d6): δ=170.62, 159.68, 153.03, 145.63, 130.74, 125.33, 120.40, 113.72, 106.80, 83.28, 71.90, 31.56, 31.38, 30.68, 28.30, 12.74. HR-MS: Calculated for [C₁₆H₁₆N₆O₃]⁺, 326.1253; found 326.1249.

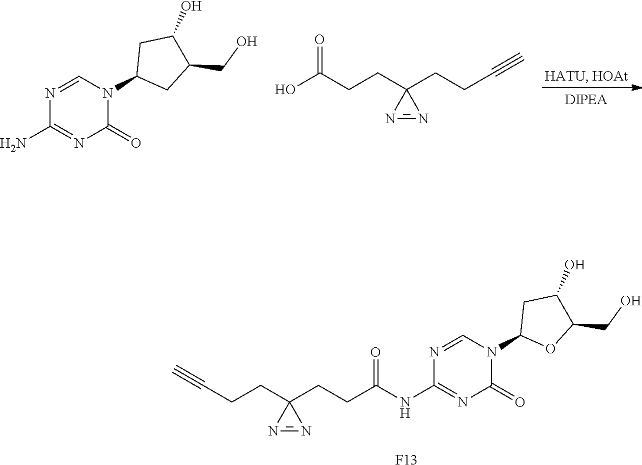

Scheme 13: Compound F13 was synthesized as described in General Procedure for Diazirine Probe Synthesis and Purification.

Characterization of F13. F13 was obtained in 27% yield (1.8 mg, 4.8 μmol). 1H NMR (400 MHz, CD3OD): δ=8.38 (s, 1H), 6.09 (t, J=8 Hz, 1H), 4.41-4.32 (m, 2H), 4.14 (q, J=4 Hz, 1H), 3.82 (t, J=4 Hz, 1H), 2.32-2.24 (m, 2H), 2.25-2.18 (m, 2H), 2.05-2.00 (m, 2H), 1.84-1.75 (m, 2H), 1.66-1.58 (m, 3H). ¹³C NMR (125 MHz, DMSO-d6): δ −172.15, 171.95, 166.33, 156.55, 85.99, 84.60, 83.87, 72.43, 70.65, 61.68, 38.20, 31.70, 28.40, 28.20, 27.80, 13.06. HR-MS: Calculated for [C₁₆H₂₁N₆O₅]⁺, 377.1573; found 377.1570.

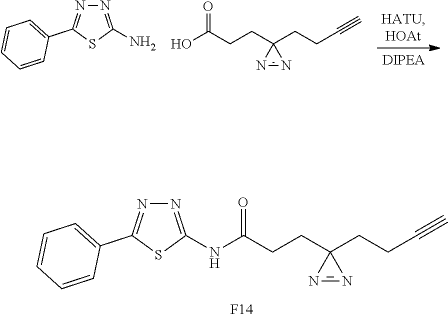 [SMILES: Nc1nnc(-c2ccccc2)s1 ; C#CCCC1(CCC(=O)O)N=N1 ; C#CCCC1(CCC(=O)Nc2nnc(-c3ccccc3)s2)N=N1 ; "F14"]

Scheme 14: Compound F14 was synthesized as described in General Procedure for Diazirine Probe Synthesis and Purification.

Characterization of F14. F14 was obtained in 55% yield (3.2 mg, 9.8 μmol). 1H NMR (600 MHz, DMSO-d6): δ=12.66 (br, 1H), 7.96-7.91 (m, 2H), 7.55-7.50 (m, 3H), 2.83 (t. J=6 Hz, 2H), 2.36 (t, J=6 Hz, 2H), 2.02 (td, J=3-6, Hz, 2H), 1.81 (t, J=6 Hz, 1H), 1.61 (t, J=6 Hz, 2H). ¹³C NMR (600 MHZ, DMSO-d6): δ=172.15, 171.95, 166.33, 156.55, 85.99, 84.60, 83.87, 72.43, 70.65, 61.68, 38.20, 31.70, 28.40, 28.20, 27.80, 13.06. HR-MS: Calculated for [C₁₆H₁₆N₅OS]⁺, 326.1076; found 326.1073.

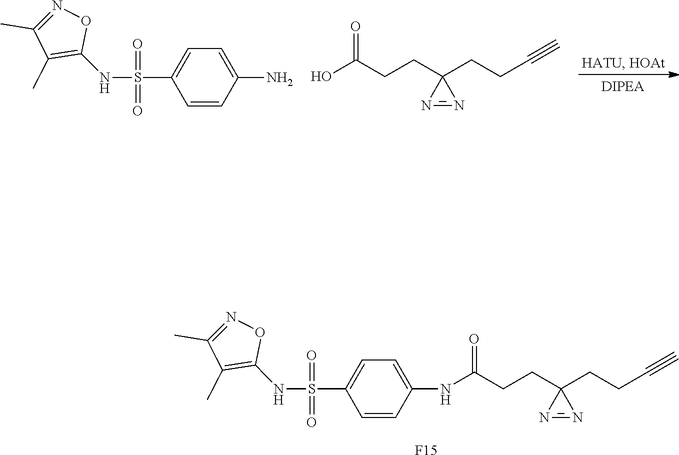

Scheme 15: Compound F15 was synthesized as described in General Procedure for Diazirine Probe Synthesis and Purification.

Characterization of F15. F15 was obtained in 35% yield (1.2 mg, 2.9 μmol). 1H NMR (600 MHz, DMSO-d6): δ=10.93 (br, 1H), 10.37 (s, 1H), 7.76 (m, 2H), 7.69 (m, 2H), 2.82 (t. J=3 Hz, 1H), 2.19 (t, J=8 Hz, 2H), 2.07 (s, 3H), 2.01 (td, J=3-7, 2H) 1.77 (t, J=8 Hz, 2H), 1.60 (m, 5H); ¹³C NMR (600 MHZ, DMSO-d6): δ=170.64, 161.52, 155.60, 143.45, 133.60, 128.02, 118.86, 105.21, 83.26, 71.86, 31.56, 30.64, 28.28, 27.57, 12.74, 10.40, 5.92. HR-MS: Calculated for [C₁₀H₂₂N₅O₄S]⁺, 416.1393; found 416.1390.

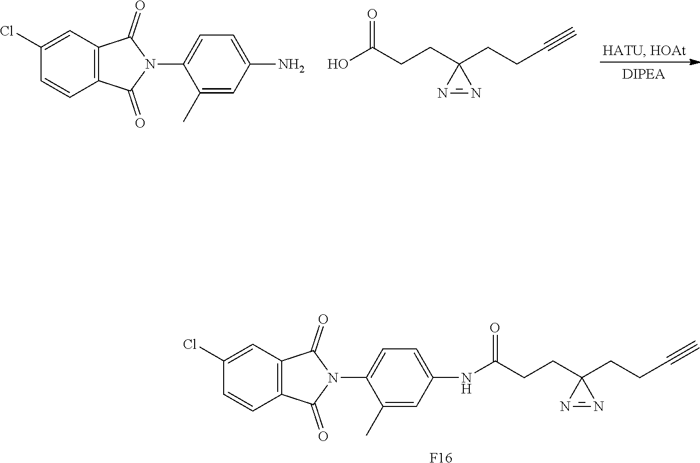 [SMILES: Cc1cc(N)ccc1N1C(=O)c2ccc(Cl)cc2C1=O ; C#CCCC1(CCC(=O)O)N=N1 ; C#CCCC1(CCC(=O)Nc2ccc(N3C(=O)c4ccc(Cl)cc4C3=O)c(C)c2)N=N1 ; "F16"]

Scheme 16: Compound F16 was synthesized as described in General Procedure for Diazirine Probe Synthesis and Purification.

Characterization of F16. F16 was obtained in 42% yield (2.1 mg, 4.8 μmol). 1H NMR (400 MHZ, CD3OD): δ=7.92-7.87 (m, 2H), 7.82 (dd, J=8-2 Hz, 1H) 7.63 (d, J=2 Hz, 1H), 7.51 (dd, J=8-2 Hz, 1H), 7.12 (d, J=8 Hz, 1H), 2.19 (m, 2H), 2.14 (s, 3H), 2.11 (t, J=4 Hz, 1H), 2.02 (td, J=6-2, 2H), 1.86 (m, 2H), 1.66 (t, J=6 Hz, 2H). ¹³C NMR (600 MHZ, DMSO-d6): δ=170.02, 166.49, 166.10, 139.90, 139.62, 136.79, 134.58, 133.74, 130.31, 129.56, 125.52, 125.38, 123.75, 120.87, 117.33, 83.27, 71.87, 31.56, 30.58, 28.33, 27.80, 17.74, 12.75. HR-MS: Calculated for [C₂H₂₀N₄O₃Cl]⁺. 435.1224; found 435.1228.

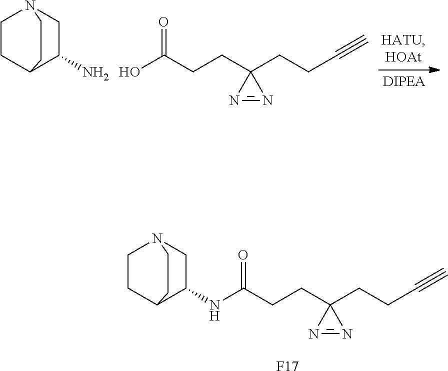 [SMILES: N[C@H]1CN2CCC1CC2 ; C#CCCC1(CCC(=O)O)N=N1 ; C#CCCC1(CCC(=O)N[C@H]2CN3CCC2CC3)N=N1 ; "F17"]

Scheme 17: Compound F17 was synthesized as described in General Procedure for Diazirine Probe Synthesis and Purification.

Characterization of F17. F17 was obtained in 95% yield (3.1 mg, 11 μmol). 1H NMR (400 MHz, CDCl₃): δ=7.76 (d. J=8 Hz, 1H), 4.47 (s, 2H), 4.38 (dd, J=8-16 Hz, 1H), 3.62-3.52 (m, 1H), 3.52-3.41 (m, 2H), 3.31-3.11 (m, 3H), 2.37 (q, J=4 Hz, 1H), 2.30-2.19 (m, 1H), 2.07-1.99 (m, 6H), 1.85-1.80 (m, 2H), 1.64 (t, J=8 Hz, 2H). ¹³C NMR (600 MHz, DMSO-d6): δ=171.15, 83.24, 71.85, 53.62, 51.94, 45.71, 45.11, 43.79, 31.46, 29.41, 28.32, 27.99, 23.96, 21.43, 12.72. HR-MS: Calculated for [C₁₅H₂₃N₄O]⁺, 275.1871; found 275.1874.

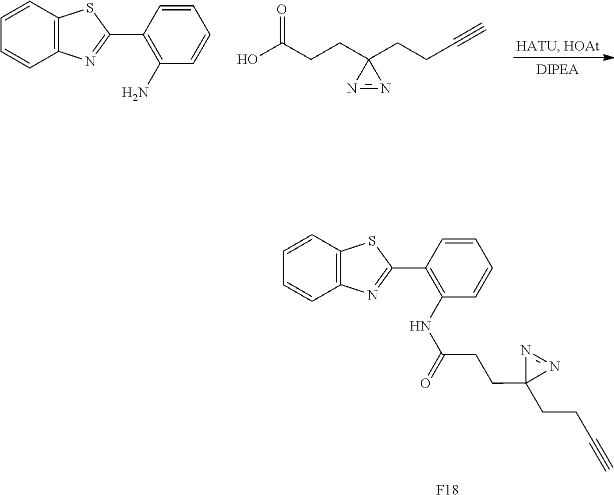 [SMILES: Nc1ccccc1-c1nc2ccccc2s1 ; C#CCCC1(CCC(=O)O)N=N1 ; C#CCCC1(CCC(=O)Nc2ccccc2-c2nc3ccccc3s2)N=N1 ; "F18"]

Scheme 18: Compound F18 was synthesized as described in General Procedure for Diazirine Probe Synthesis and Purification.

Characterization of F18. F18 was obtained in 53% yield (4.0 mg, 10.7 μmol). 1H NMR (400 MHZ, CDCl₃): δ=12.49 (s, 1H), 8.77 (m, 1H), 8.04 (m, 1H), 7.94 (m, 1H), 7.87 (m, 1H), 7.56 (m, 1H), 7.51-7.41 (m, 2H), 7.17 (m, 1H), 2.37 (m, 2H), 2.07 (td, J=8-3 Hz, 2H), 2.01 (m, 2H), 1.98 (t, J=3 Hz, 1H), 1.73 (t, J=7 Hz, 2H). ¹³C NMR (600 MHz, DMSO-d6): δ=170.42, 167.50, 152.33, 137.03, 133.38, 132.00, 130.19, 127.04, 126.17, 124.21, 122.72, 122.27, 121.86, 120.66, 83.23, 71.85, 31.58, 31.44, 28.26, 27.93, 12.75. HR-MS: Calculated for [C₂₁H₁₉N₄OS]⁺, 375.1280; found 375.1277.

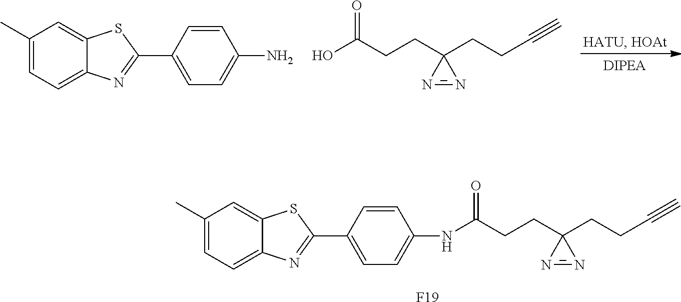

Scheme 19: Compound F19 was synthesized as described in General Procedure for Diazirine Probe Synthesis and Purification.

Characterization of F19. F19 was obtained in 95% yield (8.5 mg, 21.9 μmol). 1H NMR (400 MHz, CDCl₃): δ=8.04 (m, 2H), 7.92 (d, J=8, 1H), 7.68 (s, 1H), 7.64 (m, 2H), 7.29 (m, 1H), 2.50 (s, 3H), 2.16 (m, 2H), 2.06 (td, J=7-3 Hz, 2H), 2.00 (t, J=3 Hz, 1H), 1.97 (m, 2H), 1.70 (t, J=7 Hz, 2H). ¹³C NMR (600 MHz, DMSO-d6): δ=170.26, 165.94, 151.84, 141.83, 135.10, 134.48, 128.10, 127.92, 127.68, 122.21, 121.85, 119.29, 83.27, 71.87, 31.58, 30.65, 28.32, 27.71, 21.12, 12.75. HR-MS: Calculated for [C₂₂H₂₁N₄OS]⁺, 389.1436; found 389.1439.

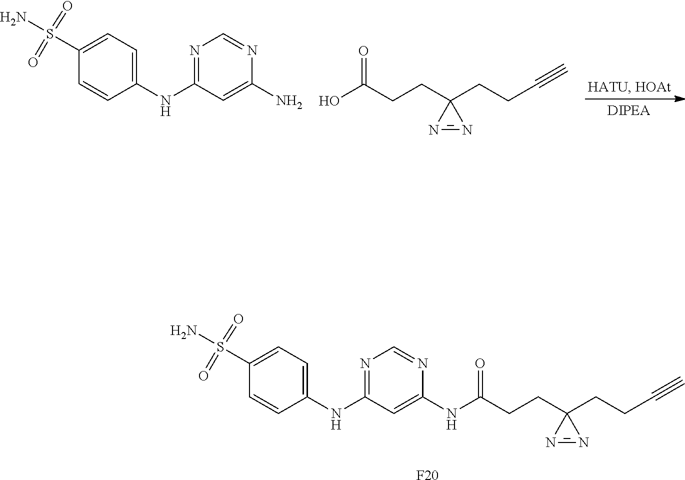 [SMILES: Nc1cc(Nc2ccc(S(N)(=O)=O)cc2)ncn1 ; C#CCCC1(CCC(=O)O)N=N1 ; C#CCCC1(CCC(=O)Nc2cc(Nc3ccc(S(N)(=O)=O)cc3)ncn2)N=N1 ; "F20"]

Scheme 20: Compound F20 was synthesized as described in General Procedure for Diazirine Probe Synthesis and Purification.

Characterization of F20. F20 was obtained in 9% yield (1.1 mg, 2.7 μmol). 1H NMR (400 MHz, CD3OD): δ=8.49 (s, 1H), 7.88 (m, 2H), 7.81 (m, 2H), 7.27 (s, 1H), 2.32 (t, J=7 Hz, 2H), 2.28 (t, J=3, 1H), 2.05 (td, J=7-3 Hz, 2H), 1.86 (t, J=8 Hz, 2H), 1.65 (t, J=7 Hz, 2H). 13C NMR (600 MHz, DMSO-d6): δ=171.93, 161.25, 157.72, 156.92, 143.42, 136.76, 126.75, 118.64, 93.97, 83.28, 71.91, 31.58, 30.46, 28.27, 27.56, 12.73. HR-MS: Calculated for [C₁₈H₂₀N₇O₃S]⁺, 414.1348; found 414.1344.

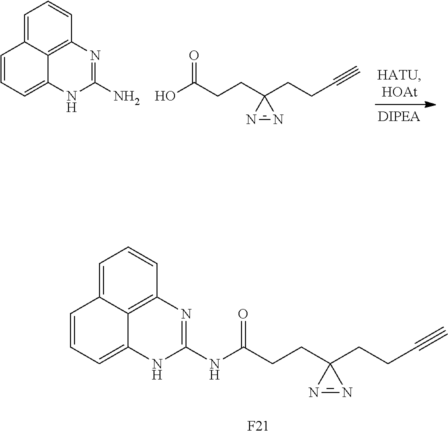 [SMILES: NC1=Nc2cccc3cccc(c23)N1 ; C#CCCC1(CCC(=O)O)N=N1 ; C#CCCC1(CCC(=O)NC2=Nc3cccc4cccc(c34)N2)N=N1 ; "F21"]

Scheme 21: Compound F21 was synthesized as described in General Procedure for Diazirine Probe Synthesis and Purification.

Characterization of F21. F21 was obtained in 80% yield (3.1 mg, 9.4 μmol). 1H NMR (400 MHz, CD3OD): δ=7.13 (t, J=4 Hz, 2H), 7.07 (d, J=4 Hz, 2H), 6.48 (d. J=4 Hz, 2H), 2.28 (t, J=2 Hz, 1H), 2.24 (t, J=4 Hz, 2H), 2.05 (td, J=4-2 Hz, 2H), 1.82 (t, J=5 Hz, 2H), 1.64 (t, J=5 Hz, 2H). ¹³C NMR (600 MHz, CD3OD): δ=178.90, 150.76, 136.69, 129.22, 121.47, 120.38, 108.66, 83.62, 70.34, 34.44, 32.56, 29.02, 28.82, 13.85. HR-MS: Calculated for [C₁₉H₁₈N₅O₁]⁺, 332.1511; found 332.1508.

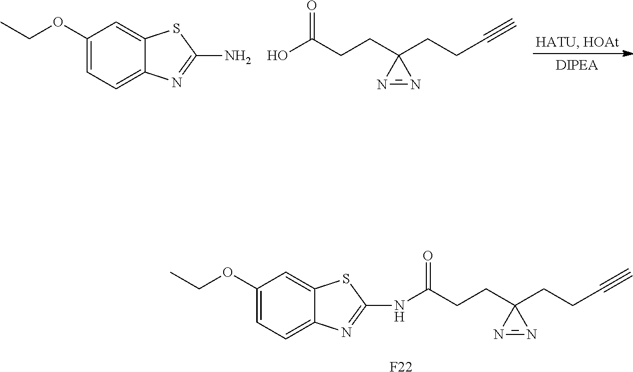 [SMILES: CCOc1ccc2nc(N)sc2c1 ; C#CCCC1(CCC(=O)O)N=N1 ; C#CCCC1(CCC(=O)Nc2nc3ccc(OCC)cc3s2)N=N1 ; "F22"]

Scheme 22: Compound F22 was synthesized as described in General Procedure for Diazirine Probe Synthesis and Purification.

Characterization of F22. F22 was obtained in 73% yield (2.8 mg, 8.2 μmol). 1H NMR (400 MHZ, CDCl₃): δ=7.63 (d, J=8 Hz, 1H), 7.28 (d, J=4 Hz, 1H), 7.06 (dd, J=8-4 Hz, 1H), 4.09 (q, J=8 Hz, 2H), 2.27 (t, J=8 Hz, 2H), 2.04-1.91 (m, 5H), 1.63 (t, J=8 Hz, 2H), 1.45 (t, J=8 Hz, 2H). ¹³C NMR (600 MHz, DMSO-d6): δ=170.66, 155.79, 155.39, 142.55, 132.77, 121.18, 115.30, 105.40, 83.24, 71.88, 63.66, 31.45, 29.44, 28.19, 27.39, 14.77, 12.72. HR-MS: Calculated for [C₁₇H₁₉N₄O₂S]⁺, 343.1229; found 343.1232.

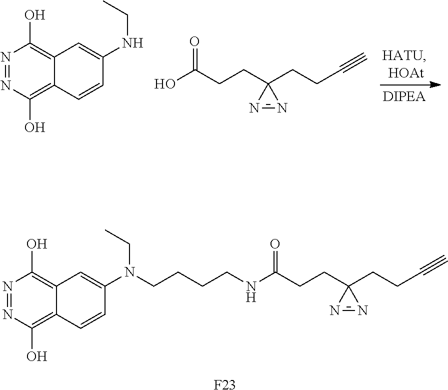 [SMILES: CCNc1ccc2c(O)nnc(O)c2c1 ; C#CCCC1(CCC(=O)O)N=N1 ; C#CCCC1(CCC(=O)NCCCCN(CC)c2ccc3c(O)nnc(O)c3c2)N=N1 ; "F23"]

Scheme 23: Compound F23 was synthesized as described in General Procedure for Diazirine Probe Synthesis and Purification.

Characterization of F23. F23 was obtained in 42% yield (1.8 mg, 4.2 μmol). 1H NMR (600 MHz, DMSO-d6): δ=7.88 (t, J=4 Hz, 1H), 7.82 (d, J=8 Hz, 1H), 7.16 (dd, J=4-8 Hz, 1H), 7.01 (br, 1H), 3.47 (peak under solvent, 2H), 3.38 (t peak under solvent, J=8 Hz, 2H), 3.07 (q, J=4 Hz, 2H), 2.80 (t, J=4 Hz, 1H), 1.96 (td, J=4-8 Hz, 2H), 1.87 (dd, J=2-8 Hz, 2H), 1.63 (dd, J=2-8 Hz, 2H), 1.55 (m, 4H), 1.45 (quint, J=7 Hz, 2H), 1.12 (t, J=4 Hz, 2H); ¹³C NMR (600 MHz, DMSO-d6): δ=170.66, 158.44, 158.20, 150.38, 128.70, 127.10, 116.68, 115.12, 103.27, 83.25, 71.79, 49.39, 44.52, 40.43, 38.30, 31.51, 29.58, 28.34, 28.19, 26.63, 24.28, 12.72, 11.90. HR-MS: Calculated for [C₂₂H₂₉N₆O₃]⁺, 425.2301; found 425.2305.

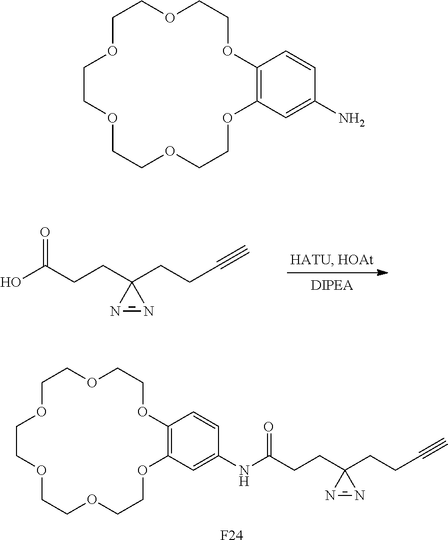 [SMILES: Nc1ccc2c(c1)OCCOCCOCCOCCOCCO2 ; C#CCCC1(CCC(=O)O)N=N1 ; C#CCCC1(CCC(=O)Nc2ccc3c(c2)OCCOCCOCCOCCOCCO3)N=N1 ; "F24"]

Scheme 24: Compound F24 was synthesized as described in General Procedure for Diazirine Probe Synthesis and Purification.

Characterization of F24. F24 was obtained in 54% yield (1.2 mg, 2.5 μmol). 1H NMR (400 MHz, CD3OD): δ=7.28 (d, J=2 Hz, 1H), 6.99 (dd, J=9-2 Hz, 1H), 6.88 (d, J=9 Hz, 1H), 4.16-4.07 (m, 4H), 3.90-3.81 (m, 4H), 3.74-3.70 (m, 4H), 3.70-3.66 (m, 4H), 3.66-3.60 (m, 4H), 2.27 (t, J=3 Hz, 1H), 2.18 (m, 2H), 2.04 (td, J=8-3 Hz, 2H), 1.83 (m, 2H), 1.64 (t, J=8 Hz, 2H). ¹³C NMR (600 MHz, DMSO-d6): δ=169.28, 148.00, 144.13, 132.94, 113.55, 111.31, 105.54, 83.27, 71.85, 69.92, 69.90, 60.87, 69.85, 68.88, 68.75, 68.42, 68.10, 31.56, 30.48, 28.34, 27.90, 12.74. HR-MS: Calculated for [C₂₄H₃₄N₃O₇]⁺, 476.2397; found 476.2392.

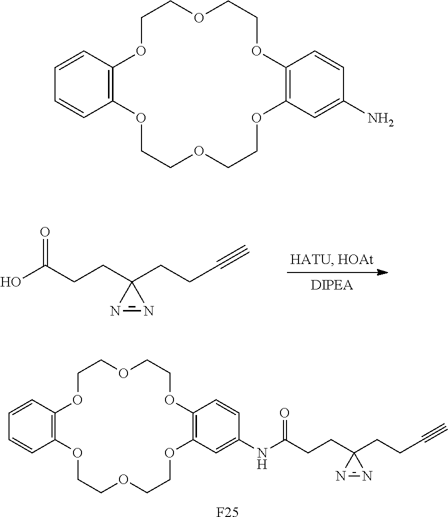

Scheme 25: Compound F25 was synthesized as described in General Procedure for Diazirine Probe Synthesis and Purification.

Characterization of F25. F25 was obtained in 8% yield (1.6 mg, 3.1 μmol). 1H NMR (400 MHZ, CD3OD): δ=7.27 (d, J=2 Hz, 1H), 6.98 (dd, J=9-2 Hz, 1H), 6.95-6.90 (m, 2H), 6.90-6.85 (m, 3H), 4.18-4.09 (m, 8H), 4.00-3.91 (m, 8H), 2.27 (t, J=3 Hz, 1H), 2.18 (m, 2H), 2.04 (td, J=8-3 Hz, 2H), 1.82 (m, 2H), 1.64 (t, J=8 Hz, 2H). ¹³C NMR (600 MHz, DMSO-d6): δ=169.27, 147.98, 147.62, 143.88, 132.70, 120.79, 112.45, 111.04, 104.75, 83.33, 71.90, 69.04, 69.00, 68.96, 68.93, 67.85, 67.68, 67.63, 67.60, 56.09, 31.55, 30.49, 28.32, 27.94, 12.74. HR-MS: Calculated for [C₂₈H₃₄N₃O₇]⁺, 524.2397; found 524.2390.

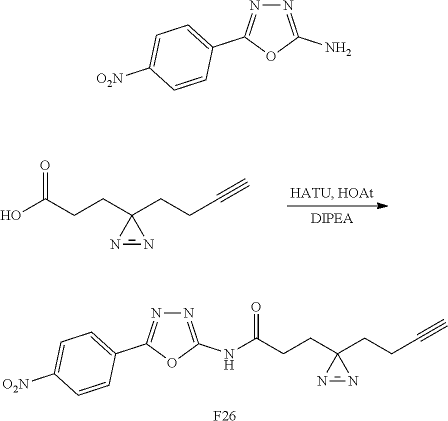 [SMILES: Nc1nnc(-c2ccc([N+](=O)[O-])cc2)o1 ; C#CCCC1(CCC(=O)O)N=N1 ; C#CCCC1(CCC(=O)Nc2nnc(-c3ccc([N+](=O)[O-])cc3)o2)N=N1 ; "F26"]

Scheme 26: Compound F26 was synthesized as described in General Procedure for Diazirine Probe Synthesis and Purification.

Characterization of F26. F26 was obtained in 83% yield (1.3 mg, 3.7 μmol). 1H NMR (400 MHz, CDCl₃): δ=8.39 (m, 2H), 8.25 (m, 2H), 2.48 (m, 2H), 2.08 (m, 2H), 2.05-1.98 (m, 3H), 1.74 (t, J=7 Hz, 2H). ¹³C NMR (600 MHz, DMSO-d6): δ=173.35, 159.01, 158.14, 148.93, 129.02, 127.28, 124.80, 83.25, 71.81, 31.48, 28.12, 27.98, 27.46, 12.68. HR-MS: Calculated for [C₁₆H₁₅N₆O₄]⁺, 355.1155; found 355.1158.

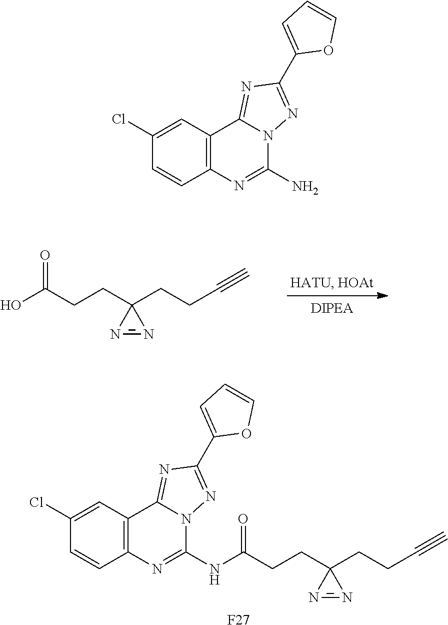

Scheme 27: Compound F27 was synthesized as described in General Procedure for Diazirine Probe Synthesis and Purification.

Characterization of F27. F27 was obtained in 25% yield (4.3 mg. 9.9 μmol). 1H NMR (400 MHz, CDCl₃): δ=8.98 (br, 1H), 8.50 (m, 1H), 7.90 (d, J=9 Hz, 1H), 7.76 (dd, J=9-2 Hz, 1H), 7.68 (dd, J=2-1 Hz, 1H), 7.31 (dd, J=4-1 Hz, 1H), 6.64 (dd, J=4-2 Hz, 1H), 2.94 (t, J=8 Hz, 2H), 2.10 (td, J=8-3 Hz, 2H), 2.07-1.98 (m, 3H), 1.77 (t, J=8 Hz, 2H). ¹³C NMR (600 MHZ, DMSO-d6): δ=170.71, 155.66, 151.33, 145.75, 144.90, 141.30, 137.87, 132.88, 131.40, 129.44, 122.42, 116.87, 113.14, 112.46, 83.30, 71.89, 31.54, 30.60, 28.26, 27.46, 12.78. HR-MS: Calculated for [C₂₁H₁₇N₇O₂Cl]⁺, 434.1132; found 434.1128.

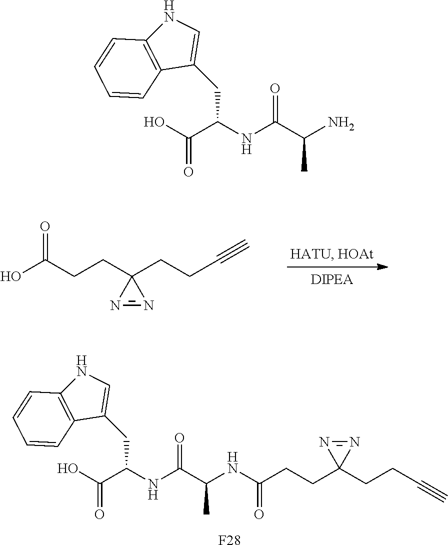

Scheme 28: Compound F28 was synthesized as described in General Procedure for Diazirine Probe Synthesis and Purification.

Characterization of F28. F28 was obtained in 61% yield (9.6 mg, 22.7 μmol). ¹H NMR (400 MHz, CD3OD): δ=7.58-7.52 (m, 1H), 7.35-7.30 (m, 1H), 7.13-7.05 (m, 2H), 7.03-6.96 (m, 1H), 4.75-4.67 (m, 1H), 4.38-4.30 (m, 1H), 3.39-3.32 (m, 1H), 3.26-3.17 (m, 1H), 2.25 (t, J=3 Hz, 1H), 2.02-1.92 (m, 4H), 1.69-1.62 (m, 2H), 1.60-1.52 (m, 2H), 1.28 (d, J=7 Hz, 2H), 1.17 (d, J=7, 1H). ¹³C NMR (600 MHz, DMSO-d6): δ=170.51, 136.09, 127.26, 123.74, 120.98, 118.45, 118.22, 111.43, 109.65, 83.28, 71.79, 52.93, 47.91, 31.40, 30.77, 29.35, 28.32, 28.20, 18.23, 12.72. HR-MS: Calculated for [C₂₂H₂₆N₅O₄]⁺, 424.1985; found 424.1981.

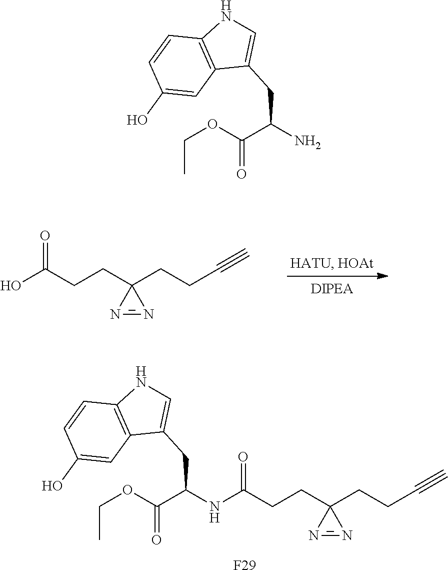 [SMILES: CCOC(=O)[C@H](N)Cc1c[nH]c2ccc(O)cc12 ; C#CCCC1(CCC(=O)O)N=N1 ; C#CCCC1(CCC(=O)N[C@H](Cc2c[nH]c3ccc(O)cc23)C(=O)OCC)N=N1 ; "F29"]

Scheme 29: Compound F29 was synthesized as described in General Procedure for Diazirine Probe Synthesis and Purification.

Characterization of F29. F29 was obtained in 95% yield (4.5 mg, 11.4 μmol). 1H NMR (400 MHz, DMSO-d6): δ=10.51 (s, 1H), 8.60 (s, 1H), 8.27 (d, J=8 Hz, 1H), 7.10 (d, J=8 Hz, 1H), 7.01 (s, 1H), 6.76 (s, 1H), 6.57 (d, J=8 Hz, 1H), 4.41 (q. J=8 Hz, 1H), 4.00 (td, J=4-8 Hz, 2H), 3.00 (dd, J=8-16 Hz, 1H), 2.89 (dd, J=8-12 Hz, 1H), 2.82-2.76 (m, 1H), 1.98-1.89 (m, 4H), 1.60-1.47 (m, 4H), 1.07 (t, J=8 Hz, 3H). ¹³C NMR (600 MHz, DMSO-d6): δ=172.05, 170.96, 150.37, 130.70, 127.80, 124.21, 111.80, 111.36, 108.46, 101.99, 83.26, 71.78, 60.44, 53.10, 31.34, 30.77, 29.20, 28.26, 28.15, 13.99, 12.70. HR-MS: Calculated for [C₂₁H₂₅N₄O₄]⁺, 397.1876; found 397.1879.

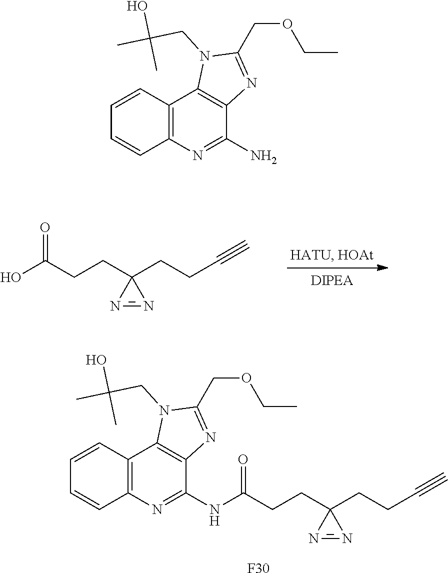 [SMILES: CCOCc1nc2c(N)nc3ccccc3c2n1CC(C)(C)O ; C#CCCC1(CCC(=O)O)N=N1 ; C#CCCC1(CCC(=O)Nc2nc3ccccc3c3c2nc(COCC)n3CC(C)(C)O)N=N1 ; "F30"]

Scheme 30: Compound F30 was synthesized as described in General Procedure for Diazirine Probe Synthesis and Purification.

Characterization of F30. F30 was obtained in 41% yield (7.5 mg, 16.2 μmol). 1H NMR (400 MHZ, CD3OD): δ=8.48 (m, 1H), 8.10 (m, 1H), 7.65 (m, 1H), 7.58 (m, 1H), 5.05 (br, 2H), 4.86 (br, 2H, overlapped with residual water), 3.63 (q, J=7 Hz, 2H), 2.61 (m, 2H), 2.28 (t, J=3 Hz, 1H), 2.08 (td, J=7-3 Hz, 2H), 1.94 (m, 2H), 1.71 (t, J=8, 2H), 1.27 (m, 9H). ¹³C NMR (600 MHz, DMSO-d6): δ=173.34, 154.00, 152.63, 149.02, 144.19, 136.58, 127.58, 124.87, 122.07, 116.96, 113.41, 83.31, 71.86, 70.75, 65.61, 64.89, 55.09, 31.59, 30.52, 28.38, 27.98, 27.80, 27.45, 15.06, 12.80. HR-MS: Calculated for [C₂₅H₃₁N₆O₃]⁺, 463.2458; found 463.2452.

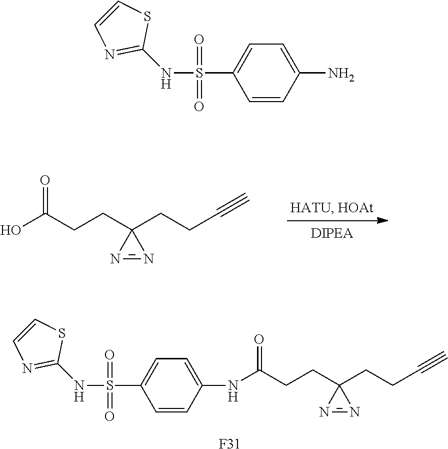

Scheme 31: Compound F31 was synthesized as described in General Procedure for Diazirine Probe Synthesis and Purification.

Characterization of F31. F31 was obtained in 22% yield (2.5 mg, 6.2 μmol). ¹H NMR (400 MHz, CD3OD): δ=7.81 (d, J=8 Hz, 2H), 7.69 (d, J=8 Hz, 2H), 7.08 (d, J=8 Hz, 1H), 6.70 (d, J=8 Hz, 1H), 2.22 (t, J=8 Hz, 2H), 2.11 (t, J=8 Hz, 2H), 1.81 (t, J=8 Hz, 1H), 1.75 (t, J=8 Hz, 2H), 1.63 (t, d, J=8 Hz, 2H). ¹³C NMR (600 MHz, DMSO-d6): δ=173.39, 170.36, 142.33, 136.33, 127.00, 118.62, 112.53, 108.20, 83.19, 71.69, 31.53, 31.48, 30.57, 27.99, 12.69. HR-MS: Calculated for [C₁₇H₁₈N₅O₃S₂]⁺, 404.0851; found 404.0855.

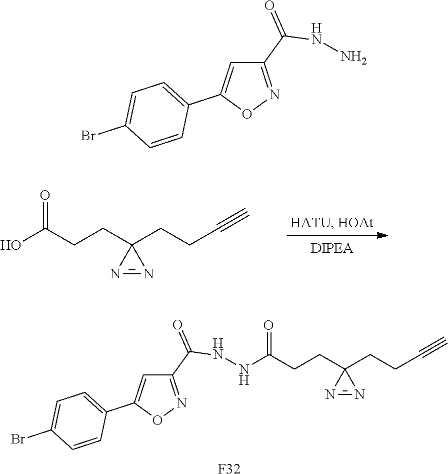 [SMILES: NNC(=O)c1cc(-c2ccc(Br)cc2)on1 ; C#CCCC1(CCC(=O)O)N=N1 ; C#CCCC1(CCC(=O)NNC(=O)c2cc(-c3ccc(Br)cc3)on2)N=N1 ; "F32"]

Scheme 32: Compound F32 was synthesized as described in General Procedure for Diazirine Probe Synthesis and Purification.

Characterization of F32. F32 was obtained in 32% yield (5.9 mg, 13.8 μmol). 1H NMR (400 MHz, DMSO-d6): δ=10.71 (s, 1H), 10.06 (s, 1H), 7.91 (m, 2H), 7.78 (m, 2H), 7.48 (s, 1H), 2.85 (t. J=3 Hz, 1H), 2.08-1.98 (m, 4H), 1.69 (m, 2H), 1.62 (t, J=7 Hz, 2H). ¹³C NMR (600 MHz, DMSO-d6): δ=170.04, 169.56, 158.33, 157.71, 132.48, 127.90, 125.39, 124.54, 100.65, 83.28, 71.87, 31.35, 28.24, 27.99, 27.45, 12.75. HR-MS: Calculated for [C₁₈H₁₇N₅O₃Br]⁺, 430.0514; found 430.0510.

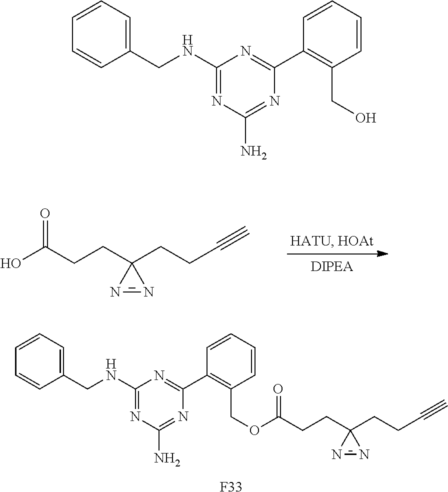 [SMILES: Nc1nc(NCc2ccccc2)nc(-c2ccccc2CO)n1 ; C#CCCC1(CCC(=O)O)N=N1 ; C#CCCC1(CCC(=O)OCc2ccccc2-c2nc(N)nc(NCc3ccccc3)n2)N=N1 ; "F33"]

Scheme 33: Compound F33 was synthesized as described in General Procedure for Diazirine Probe Synthesis and Purification.

Characterization of F33. F33 was obtained in 32% yield (4.3 mg, 9.5 μmol). ¹H NMR (600 MHz, CD3OD): δ=7.78 (m, 1H), 7.55-7.39 (m, 3H), 7.39-7.27 (m, 4H), 7.23 (m, 1H), 5.54-5.36 (m, 2H), 4.63 (s, 2H), 2.25 (m, 1H), 2.07 (m, 2H), 1.96 (m, 2H), 1.65 (m, 2H), 1.54 (m, 2H). ¹³C NMR (600 MHz, DMSO-d6): δ=173.41, 171.61, 158.21, 158.00, 140.16, 139.48, 135.41, 128.32, 127.20, 127.01, 126.89, 126.65, 115.87, 83.32, 71.86, 53.64, 45.79, 31.32, 27.98, 27.88, 27.40, 12.67. HR-MS: Calculated for [C₂₅H₂₆N₇O₂]⁺, 456.2147; found 456.2142.

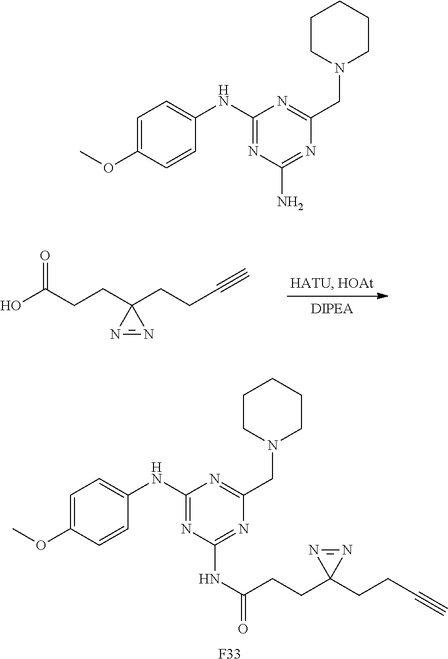

Scheme 34: Compound F34 was synthesized as described in General Procedure for Diazirine Probe Synthesis and Purification.

Characterization of F34. F34 was obtained in 28% yield (5.2 mg, 11.3 μmol). ¹H NMR (400 MHZ, CD3OD) δ (ppm): 7.75-7.40 (m, 2H), 7.05-6.76 (m, 2H), 3.79 (s, 3H), 3.55 (s, 2H), 2.78-2.57 (m, 4H), 2.56-2.34 (m, 2H), 2.26 (m, 1H), 2.10-1.96 (m, 2H), 1.90-1.23 (m, 10H). ¹³C NMR (125 MHz, DMSO-d6): δ=173.35, 171.05, 163.73, 163.15, 155.02, 129.71, 121.49, 113.35, 82.27, 71.82, 55.38, 55.22, 54.17, 35.17, 31.56, 31.13, 28.25, 27.38, 25.17, 12.74. HR-MS: Calculated for [C₂₄H₃₁N₈O₂]⁺, 463.2570; found 463.2568.

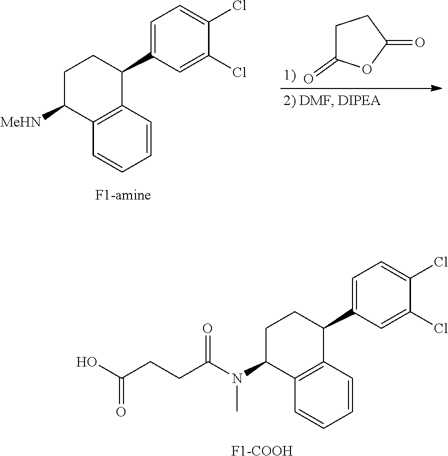

Scheme 35: Compound F1-COOH was synthesized by mixing the starting F1-amine (31 mg, 0.1 mmol) with succinic anhydride (20 mg, 0.2 mmol) in DMF (1 mL) followed by DIPEA (20 μL, 0.2 mmol) and stirred at room temperature overnight. The reaction was evaporated under vacuum to afford a crude oil. The crude oil was washed with diethyl ether twice to obtain F1-COOH as white solids.

Characterization of F1-COOH. F1-COOH was obtained in quantitative yield (38 mg, 0.1 mmol). 1H NMR (400 MHZ, CD3OD) δ (ppm): 7.42 (m, 1H), 8=7.27-7.21 (m, 2H), 7.15 (m, 2H), 6.97 (m, 2H), 5.83 (t, 1H), 4.30 (s, H), 8-2.84 (s, 3H), 8=2.72-2.65 (m, 4H), 8=2.27 (m, 1H), 8=2.06 (m, 1H), 0=1.74-1.69 (m, 2H). ¹³C NMR (600 MHz, CD3OD): δ=175.24, 173.76, 147.67, 138.35, 135.61, 131.67, 130.80, 130.44, 129.91, 128.30, 127.56, 127.38, 126.91, 126.67, 53.21, 42.75, 29.83, 28.75, 28.10, 22.08, 21.12 HR-MS: Calculated for [C₂₁H₂₂NO₃Cl₂]⁺, 406.0977; found 406.0972.

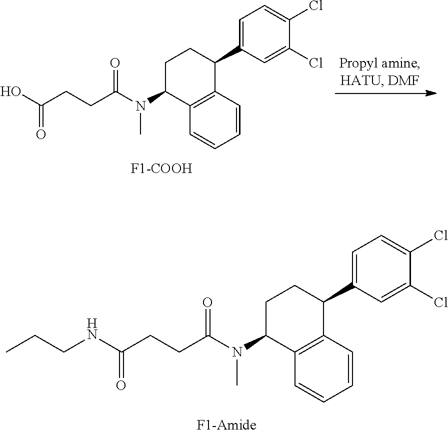

Scheme 36: Compound F1-Amide was synthesized as described below.

Synthesis of F1-Amide. Compound F1-Amide was synthesized as described in General Procedure for Diazirine Probe Synthesis and Purification, except instead of 3-(3-(but-3-yn-1-yl)-3H-diazirin-3-yl) propanoic acid, propyl amine (18 μmol, 1.5 equiv.) was used.

Characterization of F1-Amide. F1-Amide was obtained in 73% yield (3.7 mg, 8.7 μmol). 1H NMR (400 MHZ, DMSO-d6) δ (ppm): 7.82 (m, 1H), 7.55 (d, 1H), 7.29-7.20 (m, 3H), 7.06-6.95 (m, 2H), 7.82 (m, 1H), 5.70 (s, 1H), 4.32 (m, 1H), 3.00 (m, 2H), 2.73 (s, 3H), 2.40 (m, 2H), 2.23 (m, 1H), 1.97 (m, 1H), 1.57 (m, 2H), 1.40 (m, 2H), 0.42 (m, 3H). ¹³C NMR (600 MHZ, DMSO-d6) δ (ppm): 172.77, 171.76, 148.48, 138.67, 136.77, 131.30, 131.02, 130.93, 130.90 130.77, 129.34, 129.07, 127.68, 127.60, 55.91, 49.07, 42.46, 31.06, 30.07, 28.97, 22.91, 22.60, 21.63, 11.91. HR-MS: Calculated for [C₂₄H₂₉N₂O₂Cl₂]⁺, 447.1606; found 447.1610.

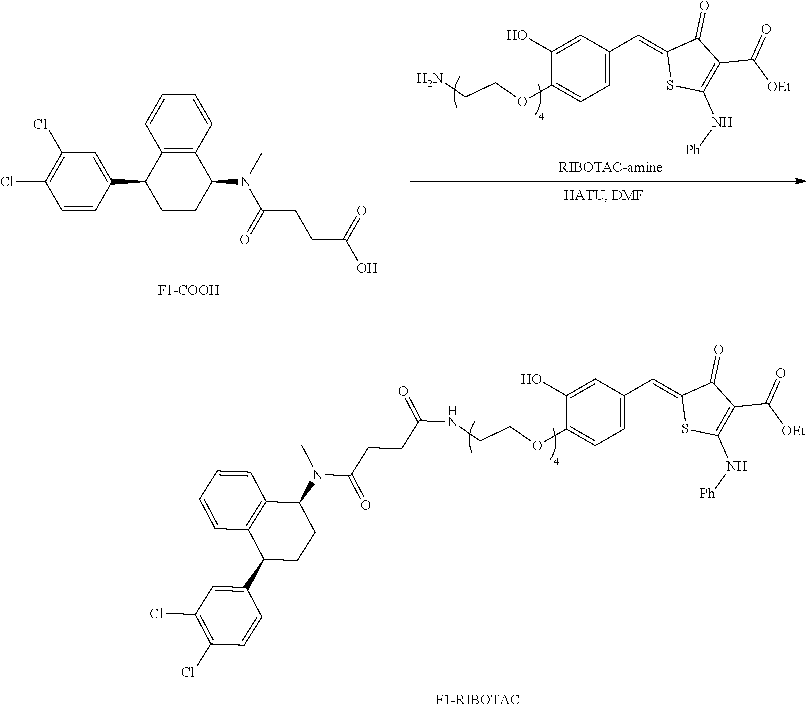

Scheme 37: Compound F1-RIBOTAC was synthesized as described in General Procedure for Diazirine Probe Synthesis and Purification, except instead of 3-(3-(but-3-yn-1-yl)-3H-diazirin-3-yl) propanoic acid, RIBOTAC-amine¹⁴ (6.3 μmol, 1.5 equiv.) was used.

Characterization of F1-RIBOTAC. F1-RIBOTAC was obtained in 43% yield (1.7 mg, 1.8 μmol). 1H NMR (600 MHZ, DMSO-d6) δ (ppm): 11.24 (s, 1H), 9.43 (s, 1H), 7.91 (m, 1H), 7.54 (m, 5H), 7.49 (m, 1H), 7.44 (m, 1H), 7.28-7.19 (m, 3H), 7.06 (m, 1H), 7.02 (m, 1H), 6.98 (m, 2H), 6.95-6.89 (m, 2H), 5.69 (s, 1H), 4.28 (m, 3H), 4.11 (t, 2H), 3.74 (t, 2H), 3.57 (m, 2H), 3.50, (m, 6H), 3.36 (2H, overlap with water), 3.20 (m, 2H), 2.71-2.65 (m, 4H), 2.43 (m, 3H), 2.21 (m, 1H), 1.95 (m, 1H), 1.72-1.56 (m, 2H), 1.29 (t, 3H). ¹³C NMR (600 MHz, DMSO-d6) δ (ppm): 180.32, 174.71, 171.63, 171.18, 170.98, 170.96, 164.25, 148.12, 147.38, 147.33, 146.41, 136.88, 129.83, 129.42, 129.01, 127.56, 126.60, 125.00, 124.01, 115.23, 113.12, 96.25, 69.31, 69.18, 69.12, 68.97, 68.53, 68.22, 67.32, 58.89, 41.37, 41.24, 37.97, 29.86, 28.97, 27.79, 21.51, 20.55, 13.79. HR-MS: Calculated for [C₄₉H₅₄N₃O₁₀Cl₂S]⁺, 946.2907; found 946.2901.

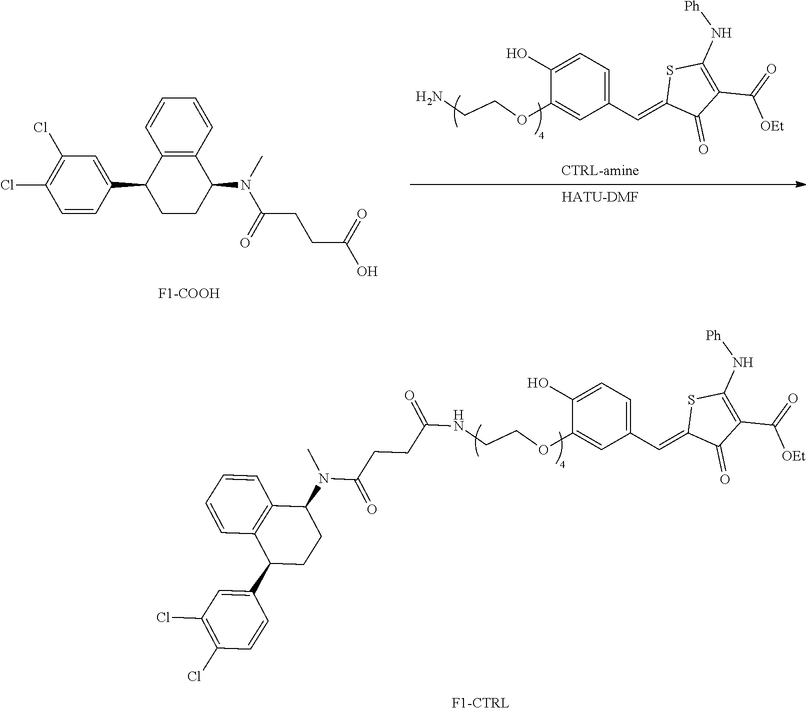

Scheme 38: Compound F1-CTRL was synthesized as described in General Procedure for Diazirine Probe Synthesis and Purification, except instead of 3-(3-(but-3-yn-1-yl)-3H-diazirin-3-yl) propanoic acid, CTRL-amine¹⁴ (6.3 μmol, 1.5 equiv.) was used.

Characterization of F1-CTRL. F1-CTRL was obtained in 38% yield (1.5 mg, 1.6 μmol). 1H NMR (600 MHz, DMSO-d6) δ (ppm): ¹H NMR (600 MHZ, DMSO-d6) δ (ppm): 11.24 (s, 1H), 9.43 (s, 1H), 7.91 (m, 1H), 7.54 (m, 5H), 7.49 (m, 1H), 7.44 (m, 1H), 7.28-7.19 (m, 3H), 7.13 (m, 1H), 7.02 (m, 1H), 6.98 (m, 1H), 6.95-6.89 (m, 2H), 6.76 (m, 1H), 5.69 (s, 1H), 4.28 (m, 3H), 4.11 (t, 2H), 3.74 (t, 2H), 3.57 (m, 2H), 3.50, (m, 6H), 3.36 (m, 2H), 3.20 (m, 2H), 2.71-2.65 (m, 4H), 2.43 (m, 3H), 2.21 (m, 1H), 1.95 (m, 1H), 1.72-1.56 (m, 2H), 1.29 (t, 3H). ¹³C NMR (600 MHZ, DMSO-d6) δ (ppm): 180.82, 175.21, 171.48, 171.18, 170.98, 170.96, 164.25, 148.62, 147.38, 146.91, 146.41, 137.38, 129.91, 129.42, 129.01, 127.01, 126.60, 125.00, 124.01, 115.23, 113.12, 96.25, 69.31, 69.18, 69.12, 68.97, 68.53, 68.22, 67.32, 58.89, 41.37, 41.24, 37.97, 29.86, 28.97, 27.79, 21.51, 20.55, 14.29. HR-MS: Calculated for [C₄₉H₅₄N₃O₁₀C₁₂S]⁺, 946.2907; found 946.2902.

### REFERENCES (EXAMPLES 2-3)

1. Disney, M. D.; Winkelsas, A. M.; Velagapudi, S. P.; Southern, M.; Fallahi, M.; Childs-Disney, J. L., Inforna 2.0: A Platform for the Sequence-Based Design of Small Molecules Targeting Structured RNAs. ACS Chem Biol 2016, 11 (6), 1720-8.
2. Morgan, B. S.; Sanaba, B. G.; Donlic, A.; Karloff, D. B.; Forte, J. E.; Zhang, Y.; Hargrove, A. E., R-BIND: An Interactive Database for Exploring and Developing RNA-Targeted Chemical Probes. ACS Chem Biol 2019, 14 (12), 2691-2700.
3. Suresh, B. M.; Li, W.; Zhang, P.; Wang, K. W.; Yildirim, I., Parker, C. G.; Disney, M. D., A general fragment-based approach to identify and optimize bioactive ligands targeting RNA. Proc Natl Acad Sci USA 2020, 117 (52), 33197-33203.
4. Rogers, D.; Hahn, M., Extended-connectivity fingerprints. J Chem Inf Model 2010, 50 (5), 742-54.
5. Parker, C. G.; Galmozzi, A.; Wang, Y.; Correia, B. E.; Sasaki, K.; Joslyn, C. M.; Kim, A. S.; Cavallaro, C. L.; Lawrence, R. M.; Johnson, S. R.; Narvaiza, I.; Saez, E.; Cravatt, B. F., Ligand and Target Discovery by Fragment-Based Screening in Human Cells. Cell 2017, 168 (3), 527-541 e29.
6. Wang, Y. J., Dix, M. M.; Bianco, G., Remsberg, J. R.; Lee, H. Y.; Kalocsay, M.; Gygi, S. P.; Forli, S.; Vite, G.; Lawrence, R. M.; Parker, C. G.; Cravatt, B. F., Expedited mapping of the ligandable proteome using fully functionalized enantiomeric probe pairs. Nature Chemistry 2019, 11 (12), 1113-1123.
7. McDowell, J. A.; Turner, D. H., Investigation of the structural basis for thermodynamic stabilities of tandem GU mismatches: solution structure of (rGAGGUCUC)2 by two-dimensional NMR and simulated annealing. Biochemistry 1996, 35 (45), 14077-89.
8. Zhang, P.; Liu, X.; Abegg, D.; Tanaka, T.; Tong, Y.; Benhamou, R. I.; Baisden, J.; Crynen, G.; Meyer, S. M.; Cameron, M. D.; Chatterjee, A. K.; Adibekian, A.; Childs-Disney, J. L.; Disney, M. D., Reprogramming of Protein-Targeted Small-Molecule Medicines to RNA by Ribonuclease Recruitment. J Am Chem Soc 2021, 143 (33), 13044-13055.
9. Dobin, A.; Davis, C. A.; Schlesinger, F.; Drenkow, J., Zaleski, C., Jha, S.; Batut, P.; Chaisson, M.; Gingeras, T. R., STAR: ultrafast universal RNA-seq aligner. Bioinformatics 2013, 29 (1), 15-21.
10. Ramirez, F.; Ryan, D. P.; Gruning, B.; Bhardwaj, V.; Kilpert, F.; Richter, A. S.; Heyne, S.; Dundar, F.; Manke, T., deepTools2: a next generation web server for deep-sequencing data analysis. Nucleic Acids Res 2016, 44 (W1), W160-5.
11. Robinson, J. T.; Thorvaldsdottir, H.; Winckler, W.; Guttman, M.; Lander, E. S.; Getz, G.; Mesirov, J. P., Integrative genomics viewer. Nat Biotechnol 2011, 29 (1), 24-6.
12. Brunner, A. D.; Thielert, M.; Vasilopoulou, C.; Ammar, C.; Coscia, F.; Mund, A.; Hoerning, O. B.; Bache, N.; Apalategui, A.; Lubeck, M.; Richter, S.; Fischer, D. S.; Raether, O.; Park, M. A.; Meier, F.; Theis, F. J.; Mann, M., Ultra-high sensitivity mass spectrometry quantifies single-cell proteome changes upon perturbation. Mol Syst Biol 2022, 18 (3), e10798.
13. Cox, J.; Mann, M., MaxQuant enables high peptide identification rates, individualized p.p.b.-range mass accuracies and proteome-wide protein quantification. Nat Biotechnol 2008, 26 (12), 1367-72.
14. Costales, M. G.; Aikawa, H.; Li, Y.; Childs-Disney, J. L.; Abegg, D.; Hoch, D. G.; Pradeep Velagapudi, S.; Nakai, Y.; Khan, T.; Wang, K. W.; Yildirim, I.; Adibekian, A.; Wang, E. T.; Disney, M. D., Small-molecule targeted recruitment of a nuclease to cleave an oncogenic RNA in a mouse model of metastatic cancer. Proc Natl Acad Sci USA 2020, 117 (5), 2406-2411.

### INCORPORATION BY REFERENCE

The present application refers to various issued patent, published patent applications, scientific journal articles, and other publications, all of which are incorporated herein by reference. The details of one or more embodiments of the invention are set forth herein Other features, objects, and advantages of the invention will be apparent from the Detailed Description, the Figures, the Examples, and the Claims.

### EQUIVALENTS AND SCOPE

In the articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present invention, as defined in the following claims.

### DESCRIPTION OF CERTAIN EMBODIMENTS

Embodiment 1. A method of transcriptome-wide mapping of RNA binding sites in a cell, comprising:
   a. Providing purified total RNA from a cell;
   b. Combining the purified total RNA with a compound comprising a diazirine moiety and an alkyne moiety to form a mixture;
   C. Irradiating the mixture;
   d Treating the irradiated mixture with a fluorescent dye comprising an azide moiety or an agarose comprising an azide moiety under conditions wherein triazolyl-bound RNA is formed;
   e. Evaluating the resulting triazolyl-bound RNA to identify a target RNA.
Embodiment 2. A method of transcriptome-wide mapping of RNA binding sites in a cell, comprising:
   a. Providing a cell;
   b. Treating the cell with a compound comprising a diazirine moiety and an alkyne moiety to form a mixture;
   c. Irradiating the mixture;
   d. Treating the irradiated mixture with a fluorescent dye comprising an azide moiety or an agarose comprising an azide moiety under conditions wherein triazolyl-bound RNA is formed; and
   e. Evaluating the resulting triazolyl-bound RNA to identify a target RNA.
Embodiment 3. The method of any one of embodiments 1 or 2, further comprising:
   f. Harvesting from the mixture total RNA comprising the triazolyl-bound RNA;
   g Fragmenting the total RNA;
   h. Performing pull-down of the triazolyl-bound RNA; and
   i. Identifying a binding site within the target RNA.
Embodiment 4. The method of any one of embodiments 1-3, wherein the cell is a cancer cell.
Embodiment 5. The method of any one of embodiments 1-4, wherein the cell is a breast cancer cell.
Embodiment 6. The method of any one of embodiments 1-5, wherein the cell is an MDA-MB-231 triple negative breast cancer cell.
Embodiment 7. The method of any one of embodiments 1-6, wherein the compound is a compound of Formula (I):

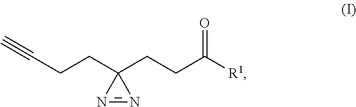

or a pharmaceutically acceptable salt thereof, wherein R¹ is selected from the group consisting of:

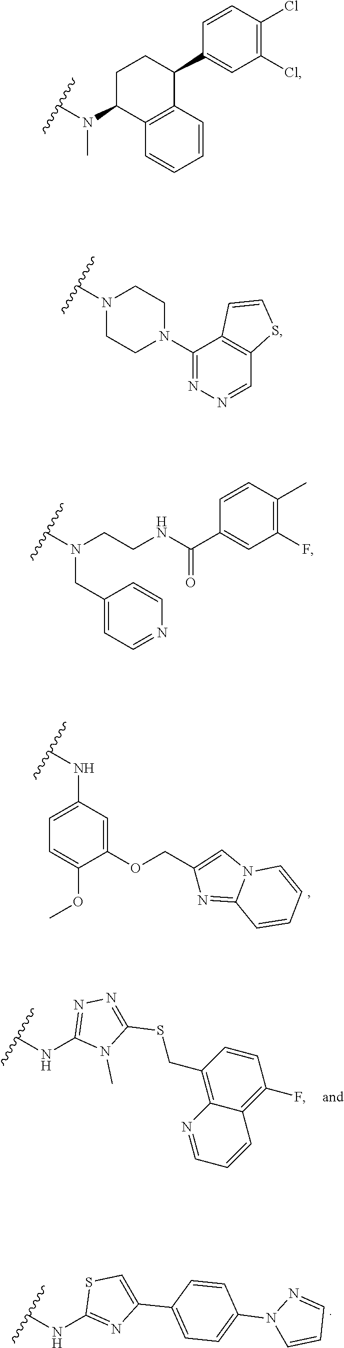 [SMILES: *N(C)[C@H]1CC[C@@H](c2ccc(Cl)c(Cl)c2)c2ccccc21 ; *N1CCN(c2nncc3sccc23)CC1 ; *N(CCNC(=O)c1ccc(C)c(F)c1)Cc1ccncc1 ; *Nc1ccc(OC)c(OCc2cn3ccccc3n2)c1 ; *Nc1nnc(SCc2ccc(F)c3cccnc23)n1C ; *Nc1nc(-c2ccc(-n3cccn3)cc2)cs1]

Embodiment 8. The method of any one of embodiments 1-7, wherein the mixture is incubated before irradiation.
Embodiment 9. The method of embodiment 8, wherein the incubation is for at least 30 minutes.
Embodiment 10. The method of any one of embodiments 8 or 9, wherein the incubation is for at least 16 hours.
Embodiment 11. The method of any one of embodiments 1-10, wherein the irradiation is for at least 10 minutes.
Embodiment 12. The method of any one of embodiments 1-11, wherein the irradiation is with ultraviolet light.
Embodiment 13. The method of any one of embodiments 1-12, wherein treating the irradiated mixture under conditions wherein triazolyl-bound RNA is formed comprises a copper (II) salt and a reducing agent.
Embodiment 14. The method of any one of embodiments 1-13, wherein treating the irradiated mixture under conditions wherein triazolyl-bound RNA is formed is for at least 3 hours.
Embodiment 15. The method of any one of embodiments 1-14, wherein evaluating the resulting triazolyl-bound RNA is by gel electrophoresis or fluorescence imaging.
Embodiment 16. The method of any one of embodiments 1-15, wherein evaluating the resulting triazolyl-bound RNA is by fluorescence imaging.
Embodiment 17. The method of any one of embodiments 1-16, wherein evaluating the resulting triazolyl-bound RNA comprises imaging the fluorescence of the fluorescent dye.
Embodiment 18. The method of any one of embodiments 1-17, wherein evaluating the resulting triazolyl-bound RNA comprises imaging tetramethylrhodamine (TAMRA) fluorescence.
Embodiment 19. The method of any one of embodiments 1-15, wherein evaluating the resulting triazolyl-bound RNA is by gel electrophoresis.
Embodiment 20. The method of any one of embodiments 1-15 or 19, further comprising treating the gel with an agent capable of staining the gel.
Embodiment 21. The method of any one of embodiments 1-15, 19, or 20, wherein the gel is stained with SYBR Green and/or Coomassie staining.
Embodiment 22. The method of any one of embodiments 1-21, wherein evaluating the resulting triazolyl-bound RNA comprises detecting a target signal at least 3-fold above a background signal.
Embodiment 23. The method of any one of embodiments 1-22, wherein evaluating the resulting triazolyl-bound RNA comprises identifying enrichment of the target RNA.
Embodiment 24. The method of any one of embodiments 1-23, wherein the method is selective for enrichment of the target RNA compared to DNA or proteins.
Embodiment 25. The method of any one of embodiments 1-24, wherein the compound comprises a moiety capable of binding RNA.
Embodiment 26. The method of any one of embodiments 1-25, wherein evaluating the resulting triazolyl-bound RNA comprises identifying a control target that non-specifically reacts with the diazirine moiety.
Embodiment 27. The method of any one of embodiments 1-26, further comprising using a control compound comprising a diazirine moiety and an alkyne moiety, wherein the control compound does not comprise a moiety capable of binding RNA, to identify a control target that non-specifically reacts with the diazirine moiety.
Embodiment 28. The method of any one of embodiments 1-27, wherein the RNA is QSOX1 mRNA.
Embodiment 29. The method of any one of embodiments 1-28, wherein the RNA is QSOX1-a mRNA.
Embodiment 30. The method of any one of embodiments 3-29, wherein fragmenting the total RNA comprises random fragmentation.
Embodiment 31. The method of any one of embodiments 3-30, wherein performing pull-down of triazolyl-bound RNA comprises selectively pulling-down fragmented RNA regions bound by the compound.
Embodiment 32. The method of any one of embodiments 3-31, wherein the concentration of the compound is sufficient for the pull-down of triazolyl-bound RNA to enrich the target RNA.
Embodiment 33. The method of embodiment 32, wherein the concentration of the compound is at least 5 μM (˜6-fold) or 20 μM (˜12-fold).
Embodiment 34. The method of any one of embodiments 3-33, wherein the pull-down of triazolyl-bound RNA is performed for a time sufficient to enrich the target RNA.
Embodiment 35. The method of any one of embodiments 3-34, wherein the pull-down of triazolyl-bound RNA is performed for at least 8 hours.
Embodiment 36. The method of any one of embodiments 3-35, wherein the pull-down of triazolyl-bound RNA is performed for at least 16 hours.
Embodiment 37. The method of any one of embodiments 3-36, wherein the method does not pull-down a protein capable of forming an mRNA-protein complex.
Embodiment 38. The method of any one of embodiments 3-37, wherein the method does not pull-down a protein produced from the target RNA.
Embodiment 39. A method of making a modified ribonucleic acid, or a pharmaceutically acceptable salt thereof, comprising reacting a ribonucleic acid with a compound of Formula (I):

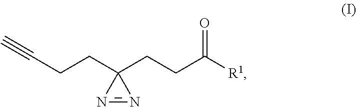

or a pharmaceutically acceptable salt thereof, wherein R¹ is selected from the group consisting of:

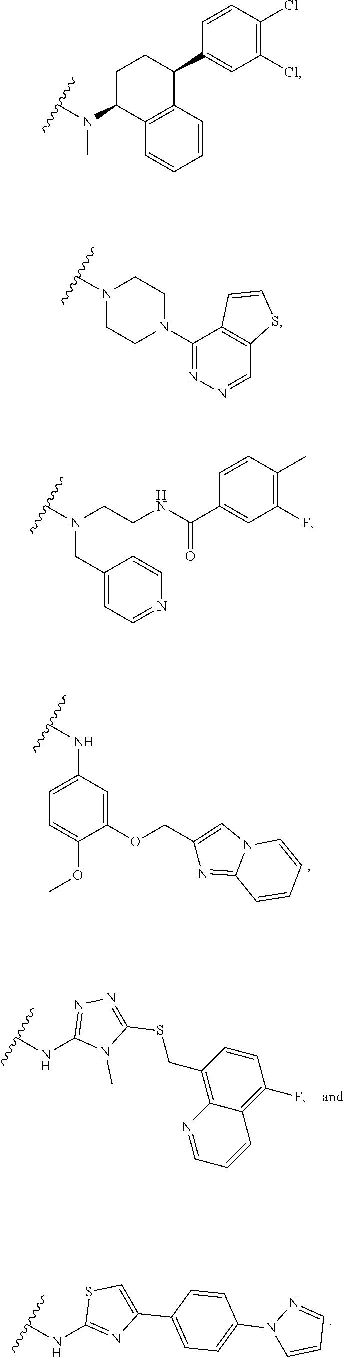 [SMILES: *N(C)[C@H]1CC[C@@H](c2ccc(Cl)c(Cl)c2)c2ccccc21 ; *N1CCN(c2nncc3sccc23)CC1 ; *N(CCNC(=O)c1ccc(C)c(F)c1)Cc1ccncc1 ; *Nc1ccc(OC)c(OCc2cn3ccccc3n2)c1 ; *Nc1nnc(SCc2ccc(F)c3cccnc23)n1C ; *Nc1nc(-c2ccc(-n3cccn3)cc2)cs1]

Embodiment 40. A modified ribonucleic acid, or a pharmaceutically acceptable salt thereof, made by the method of embodiment 39.
Embodiment 41. A method of making a fluorescent-tagged ribonucleic acid, or a pharmaceutically acceptable salt thereof, comprising reacting the modified ribonucleic acid of embodiment 40, or a pharmaceutically acceptable salt thereof, with a fluorescent dye comprising an azide moiety.
Embodiment 42. A method of making a modified agarose, comprising reacting the modified ribonucleic acid of embodiment 40, or a pharmaceutically acceptable salt thereof, with an agarose comprising an azide moiety.
Embodiment 43. A compound of Formula (I):

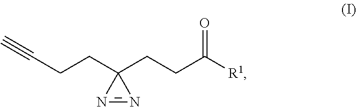

or a pharmaceutically acceptable salt thereof, wherein R¹ is selected from the group consisting of:

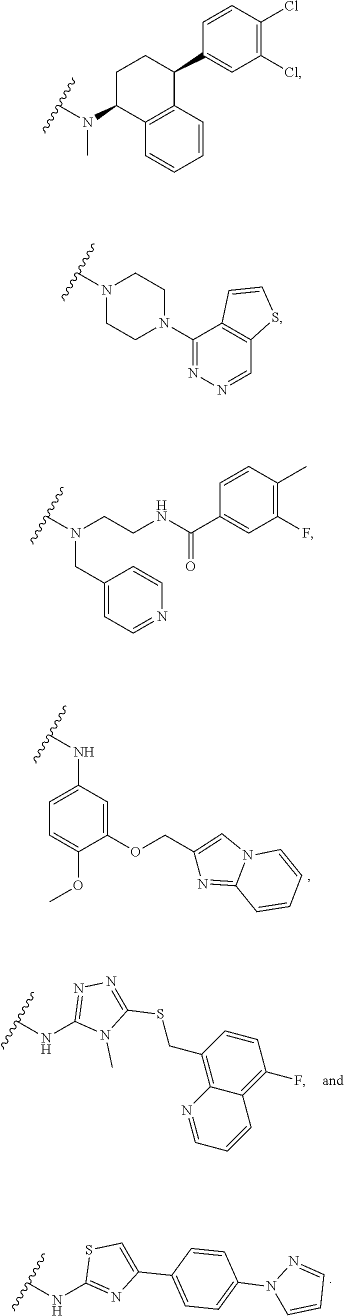 [SMILES: *N(C)[C@H]1CC[C@@H](c2ccc(Cl)c(Cl)c2)c2ccccc21 ; *N1CCN(c2nncc3sccc23)CC1 ; *N(CCNC(=O)c1ccc(C)c(F)c1)Cc1ccncc1 ; *Nc1ccc(OC)c(OCc2cn3ccccc3n2)c1 ; *Nc1nnc(SCc2ccc(F)c3cccnc23)n1C ; *Nc1nc(-c2ccc(-n3cccn3)cc2)cs1]

Embodiment 44. The compound of embodiment 43, wherein the compound of Formula (I) is of formula:

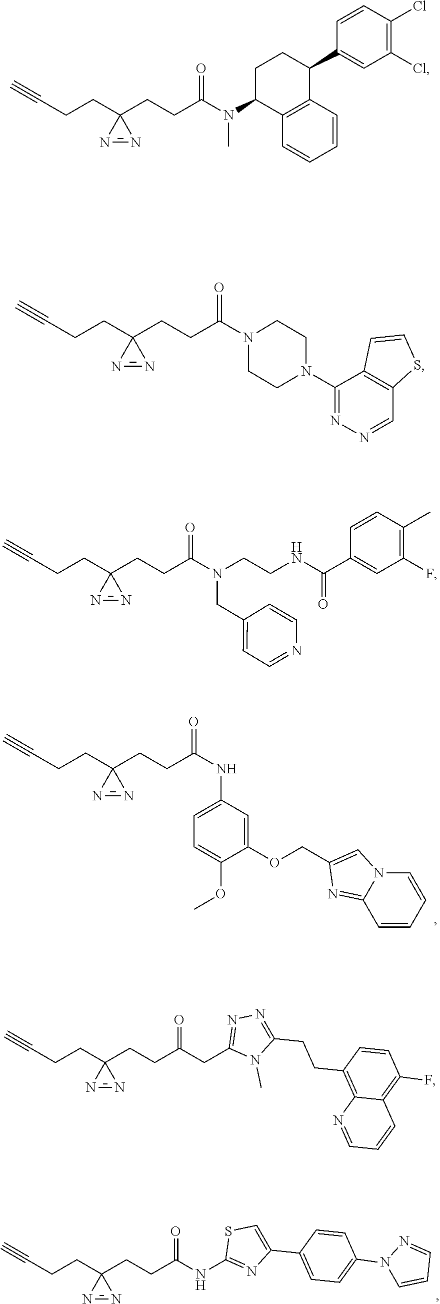 [SMILES: C#CCCC1(CCC(=O)N(C)[C@H]2CC[C@@H](c3ccc(Cl)c(Cl)c3)c3ccccc32)N=N1 ; C#CCCC1(CCC(=O)N2CCN(c3nncc4sccc34)CC2)N=N1 ; C#CCCC1(CCC(=O)N(CCNC(=O)c2ccc(C)c(F)c2)Cc2ccncc2)N=N1 ; C#CCCC1(CCC(=O)Nc2ccc(OC)c(OCc3cn4ccccc4n3)c2)N=N1 ; C#CCCC1(CCC(=O)Cc2nnc(CCc3ccc(F)c4cccnc34)n2C)N=N1 ; C#CCCC1(CCC(=O)Nc2nc(-c3ccc(-n4cccn4)cc3)cs2)N=N1]

or a pharmaceutically acceptable salt thereof.
Embodiment 45. The compound of any one of embodiments 43 or 44, wherein the compound of Formula (D) is of formula:

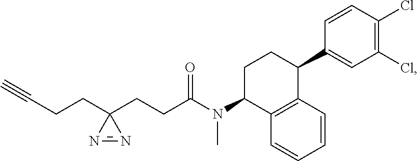 [SMILES: C#CCCC1(CCC(=O)N(C)[C@H]2CC[C@@H](c3ccc(Cl)c(Cl)c3)c3ccccc32)N=N1]

or a pharmaceutically acceptable salt thereof.
Embodiment 46. A compound of Formula (II):

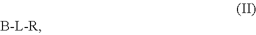

or a pharmaceutically acceptable salt thereof, wherein:
   B is an RNA binder of formula:

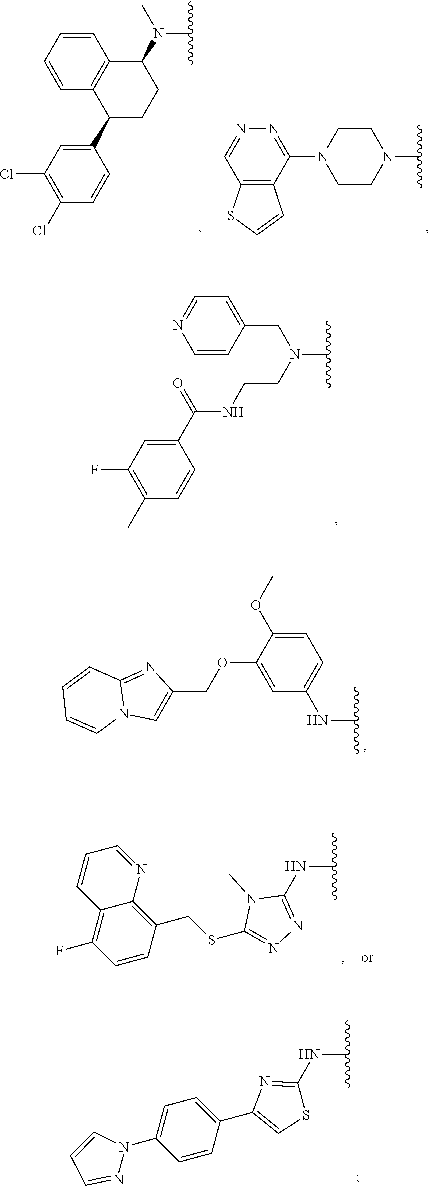 [SMILES: *N(C)[C@H]1CC[C@@H](c2ccc(Cl)c(Cl)c2)c2ccccc21 ; *N1CCN(c2nncc3sccc23)CC1 ; *N(CCNC(=O)c1ccc(C)c(F)c1)Cc1ccncc1 ; *Nc1ccc(OC)c(OCc2cn3ccccc3n2)c1 ; *Nc1nnc(SCc2ccc(F)c3cccnc23)n1C ; *Nc1nc(-c2ccc(-n3cccn3)cc2)cs1]

L is a linker, and
   R is an RNase L recruiter.
Embodiment 47. The compound of embodiment 46, or a pharmaceutically acceptable salt thereof, wherein B is an RNA binder of Formula:

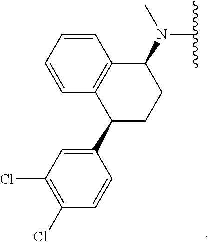

Embodiment 48. The compound of any one of embodiments 46 or 47, or a pharmaceutically acceptable salt thereof, wherein L is of Formula (III):

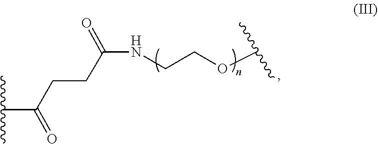

wherein n is 0, 1, 2, 3, 4, 5, 6, 7, 8, 9, or 10.
Embodiment 49. The compound of any one of embodiments 46-48, or a pharmaceutically acceptable salt thereof, wherein L is of formula:

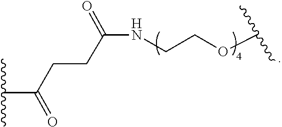

Embodiment 50. The compound of any one of embodiments 46-49, or a pharmaceutically acceptable salt thereof, wherein R is an RNAse L recruiter of Formulae (IV-a) or (IV-b):

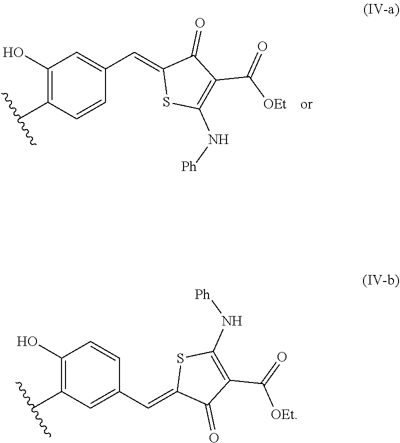 [SMILES: *c1ccc(/C=C2\SC(Nc3ccccc3)=C(C(=O)OCC)C2=O)cc1O ; *c1cc(/C=C2\SC(Nc3ccccc3)=C(C(=O)OCC)C2=O)ccc1O]

Embodiment 51. The compound of any one of embodiments 46-50, or a pharmaceutically acceptable salt thereof, wherein R is an RNAse L recruiter of Formula (IV-a):

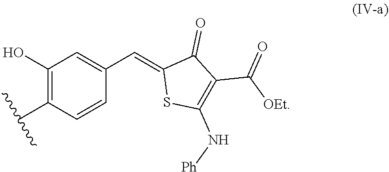 [SMILES: *c1ccc(/C=C2\SC(Nc3ccccc3)=C(C(=O)OCC)C2=O)cc1O]

Embodiment 52. The compound of any one of embodiments 46-51, or a pharmaceutically acceptable salt thereof, wherein the compound is of Formula (II-a):

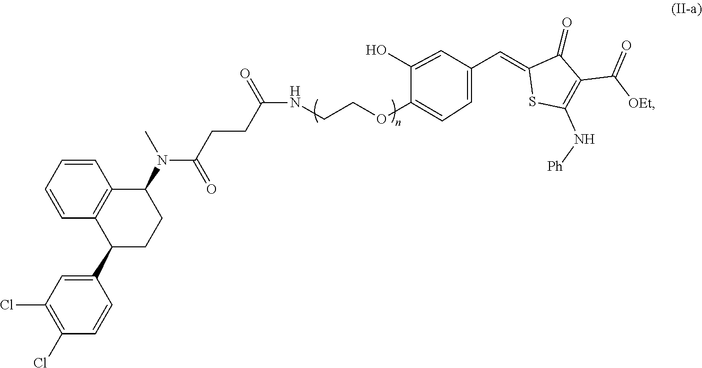

wherein n is 0, 1, 2, 3, 4, 5, 6, 7, 8, 9, or 10.
Embodiment 53. The compound of any one of embodiments 46-52, or a pharmaceutically acceptable salt thereof, wherein the compound is of formula:

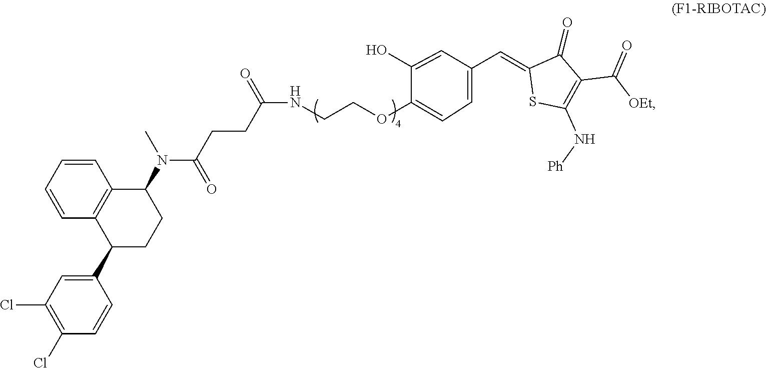

Embodiment 54. The compound of any one of embodiments 46-50, or a pharmaceutically acceptable salt thereof, wherein R is an RNAse L recruiter of Formula (IV-b):

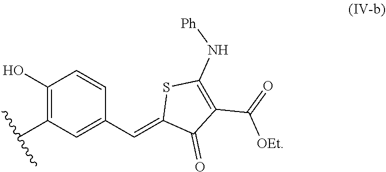 [SMILES: *c1cc(/C=C2\SC(Nc3ccccc3)=C(C(=O)OCC)C2=O)ccc1O]

Embodiment 55. The compound of any one of embodiments 46-50 or 54, or a pharmaceutically acceptable salt thereof, wherein the compound is of Formula (II-b):

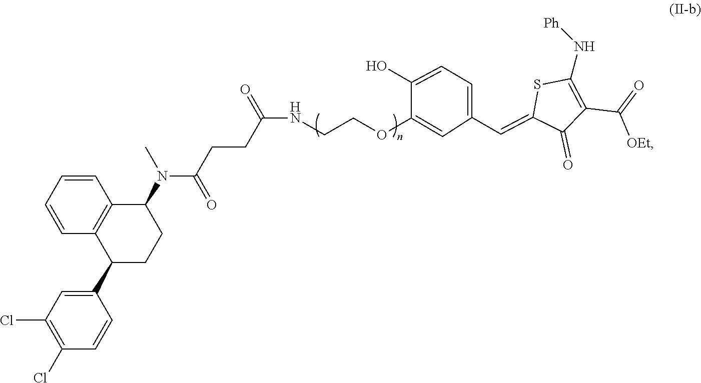

wherein n is 0, 1, 2, 3, 4, 5, 6, 7, 8, 9, or 10.
Embodiment 56. The compound of any one of embodiments 46-50, 54, or 55, or a pharmaceutically acceptable salt thereof, wherein the compound is of formula:

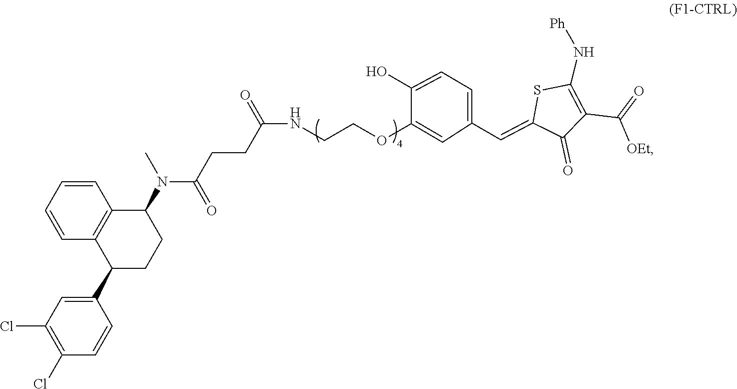

Embodiment 57. A composition comprising the compound of any one of embodiments 46-56, or a pharmaceutically acceptable salt thereof, and an excipient.
Embodiment 58. A method of binding RNase L in a subject in need thereof or in a cell, tissue, or biological sample, comprising administering to the subject in need thereof or contacting the cell, tissue, or biological sample with an effective amount of:
   a compound of any one of embodiments 46-56, or a pharmaceutically acceptable salt thereof; or
   the composition of embodiment 57.
Embodiment 59. The method of embodiment 58, wherein binding RNase L comprises activating RNase L.
Embodiment 60. The method of any one of embodiments 58 or 59, wherein binding RNase L comprises inducing RNase L dimerization.
Embodiment 61. The method of any one of embodiments 58-60, further comprising modulating QSOX1-a mRNA.
Embodiment 62. The method of any one of embodiments 58-61, further comprising degrading QSOX1-a mRNA.
Embodiment 63. A method of modulating QSOX1 mRNA in a subject in need thereof or in a cell, tissue, or biological sample, comprising administering to the subject in need thereof or contacting the cell, tissue, or biological sample with an effective amount of:
   a compound of any one of embodiments 46-56, or a pharmaceutically acceptable salt thereof; or
   the composition of embodiment 57.
Embodiment 64. The method of embodiment 63, wherein the QSOX1 mRNA is QSOX1-a mRNA.
Embodiment 65. A method of degrading a QSOX1 mRNA isoform in a subject in need thereof or in a cell, tissue, or biological sample, comprising administering to the subject in need thereof or contacting the cell, tissue, or biological sample with an effective amount of:
   a compound of any one of embodiments 46-56, or a pharmaceutically acceptable salt thereof, or
   the composition of embodiment 57.
Embodiment 66. The method of embodiment 65, wherein the QSOX1 mRNA isoform is QSOX1-a.
Embodiment 67. A method of inhibiting cell proliferation or promoting apoptosis in a subject in need thereof or in a cell, tissue, or biological sample, comprising administering to the subject in need thereof or contacting the cell, tissue, or biological sample with an effective amount of:
   a compound of any one of embodiments 46-56, or a pharmaceutically acceptable salt thereof; or
   the composition of embodiment 57.
Embodiment 68. The method of any one of embodiments 58-67, further comprising reducing an amount of QSOX1 protein.
Embodiment 69. The method of embodiment 68, wherein the QSOX1 protein is QSOX1-a.
Embodiment 70. A method of treating or preventing a disease in a subject in need thereof, comprising administering to the subject in need thereof an effective amount of:
   a compound of any one of embodiments 46-56, or a pharmaceutically acceptable salt thereof; or
   the composition of embodiment 57.
Embodiment 71. The compound of any one of embodiments 46-56, or a pharmaceutically acceptable salt thereof, or the composition of embodiment 57, for use in treating a disease in a subject in need thereof.
Embodiment 72. The compound of any one of embodiments 46-56, or a pharmaceutically acceptable salt thereof, or the composition of embodiment 57, for use in the manufacture of a medicament for treatment of a disease in a subject in need thereof.
Embodiment 73. The method, compound for use, pharmaceutically acceptable salt thereof for use, or composition for use of any one of embodiments 70-72, wherein the disease is associated with QSOX1 mRNA.
Embodiment 74. The method, compound for use, pharmaceutically acceptable salt thereof for use, or composition for use of any one of embodiments 70-73, wherein the disease is a proliferative disease.
Embodiment 75. The method, compound for use, pharmaceutically acceptable salt thereof for use, or composition for use of embodiment 74, wherein the proliferative disease is cancer.
Embodiment 76. The method, compound for use, pharmaceutically acceptable salt thereof for use, or composition for use of embodiment 75, wherein the cancer is breast cancer.
Embodiment 77. The method, compound for use, pharmaceutically acceptable salt thereof for use, or composition for use of any one of embodiments 75 or 76, wherein the cancer is triple negative breast cancer.
Embodiment 78. A method of preparing a compound of Formulae (II-a) or (II-b):

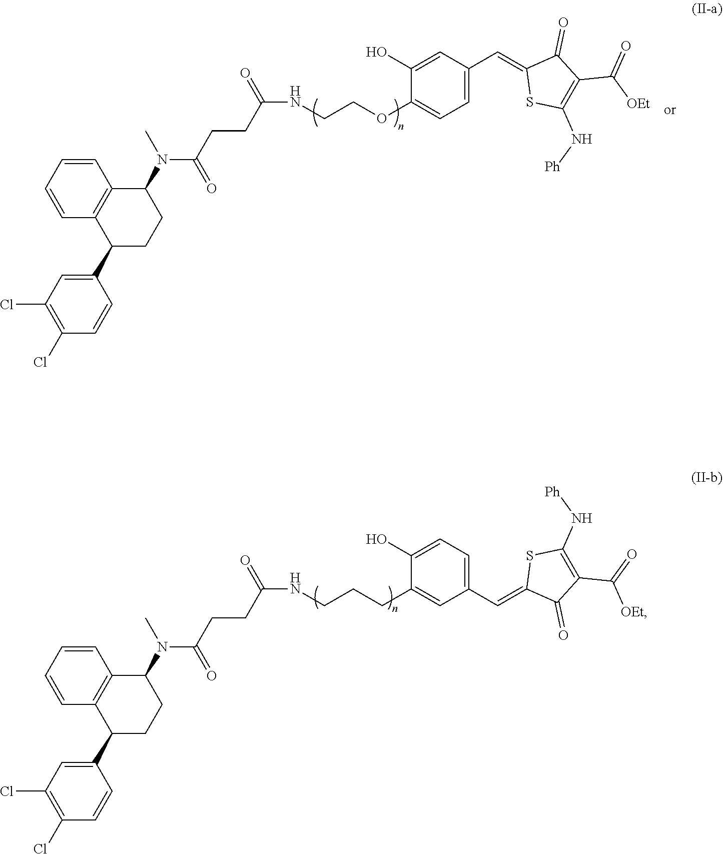

or a pharmaceutically acceptable salt thereof, comprising reacting a compound of Formulae (V-a) or (V-b):

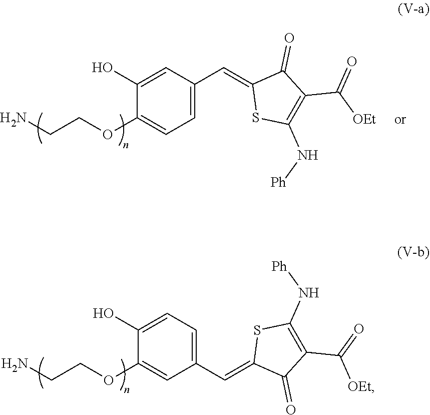

or a salt thereof, with a compound of Formula (VI):

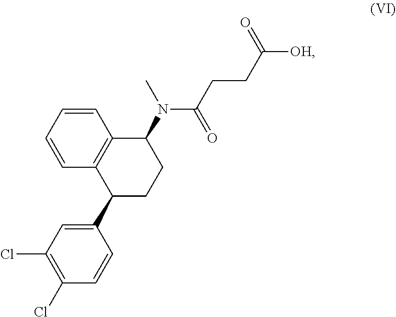 [SMILES: CN(C(=O)CCC(=O)O)[C@H]1CC[C@@H](c2ccc(Cl)c(Cl)c2)c2ccccc21]

or a salt thereof, wherein n is 0, 1, 2, 3, 4, 5, 6, 7, 8, 9, or 10.
Embodiment 79. The method of embodiment 78, further comprising alkylating a compound

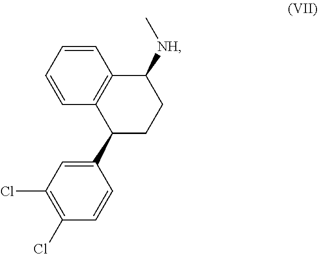 [SMILES: CN[C@H]1CC[C@@H](c2ccc(Cl)c(Cl)c2)c2ccccc21]

or a salt thereof, to provide the compound of Formula (VI), or salt thereof.
Embodiment 80. The method of any one of embodiments 78 or 79, further comprising alkylating a compound of Formulae (VIII-a) or (VIII-b):

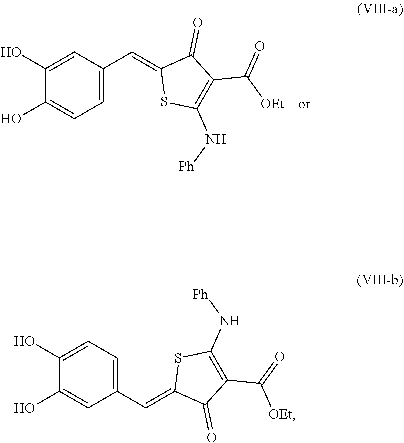 [SMILES: CCOC(=O)C1=C(Nc2ccccc2)S/C(=C\c2ccc(O)c(O)c2)C1=O ; CCOC(=O)C1=C(Nc2ccccc2)S/C(=C\c2ccc(O)c(O)c2)C1=O]

or a salt thereof, to provide the compound of Formulae (V-a) or (V-b):

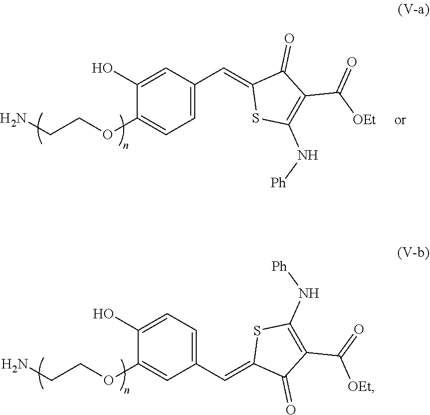

or a salt thereof.
Embodiment 81. A kit comprising the compound of any one of embodiments 46-56, or a pharmaceutically acceptable salt thereof, or the composition of embodiment 57, and instructions for its use.

### DESCRIPTION OF ADDITIONAL EMBODIMENTS

1A. A compound, or salt thereof, of Formula I:

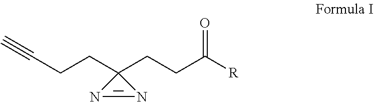

wherein
   R is selected from

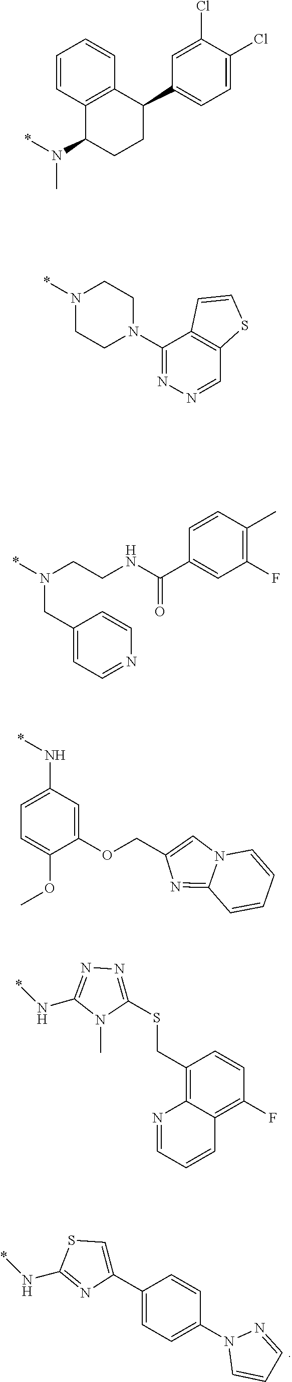 [SMILES: *N(C)[C@@H]1CC[C@H](c2ccc(Cl)c(Cl)c2)c2ccccc21 ; *N1CCN(c2nncc3sccc23)CC1 ; *N(CCNC(=O)c1ccc(C)c(F)c1)Cc1ccncc1 ; *Nc1ccc(OC)c(OCc2cn3ccccc3n2)c1 ; *Nc1nnc(SCc2ccc(F)c3cccnc23)n1C ; *Nc1nc(-c2ccc(-n3cccn3)cc2)cs1]

2A. A compound, or salt thereof, that is one of compounds F1 to F6.

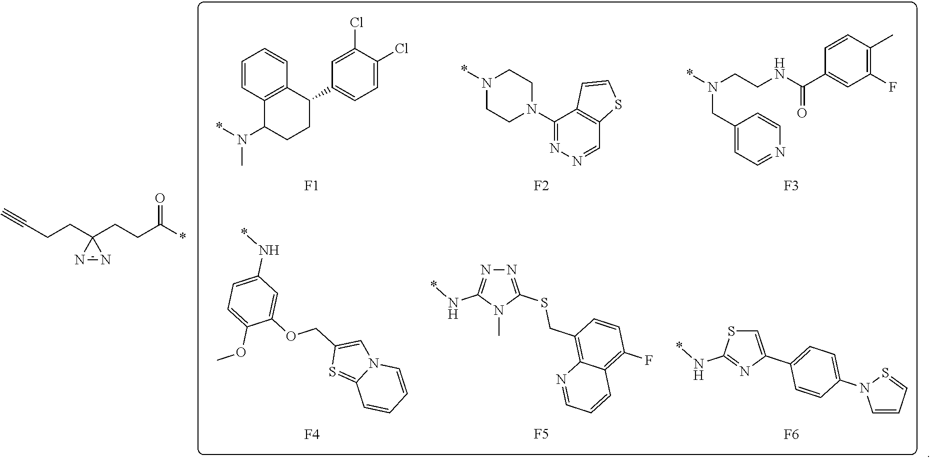

3A. The compound, or salt thereof, of embodiment 2A that is F1.
4A. The compound, or salt thereof, of embodiment 2A that is F2.
5A. The compound, or salt thereof, of embodiment 2A that is F3.
6A. The compound, or salt thereof, of embodiment 2A that is F4.
7A. The compound, or salt thereof, of embodiment 2A that is F5.
8A. The compound, or salt thereof, of embodiment 2A that is F6.
9A. A compound, or salt thereof, that is F1-RIBOTAC:

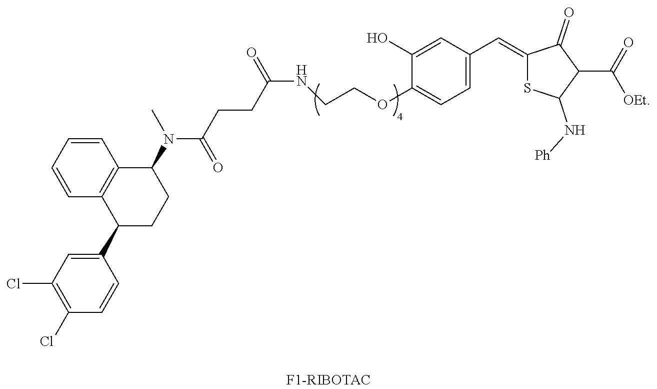

10A. A composition comprising a compound, or salt thereof, of embodiment 9A and a pharmaceutically acceptable excipient.
11A. A method of modulating QSOX1-a mRNA in a subject, comprising administration to the subject of a compound, or salt thereof, of embodiment 9A or a composition of embodiment 10A.
12A. A method of degrading a QSOX1 isoform in a subject, comprising administration to the subject of a compound, or salt thereof, of embodiment 9A or a composition of embodiment 10A.
13A. The method of embodiment 12A, wherein the isoform is QSOX1-a.
14A. A method of reducing cell proliferation in a subject, comprising administration to the subject of a compound, or salt thereof, of embodiment 9A or a composition of embodiment 10A.
15A. A method of treating cancer in a subject, comprising administration to the subject of a compound, or salt thereof, of embodiment 9A or a composition of embodiment 10A.
16A. A method of making a modified ribonucleic acid comprising reacting a ribonucleic acid with a compound of any of embodiments 1A-8A.
17A. A modified ribonucleic acid, or salt thereof, made according to the method of embodiment 16A.
18A. A method of making a fluorescent-tagged modified ribonucleic acid comprising reacting a modified ribonucleic acid of embodiment 17A with an azide-functionalized fluorescent dye.
19A. A method of making an agarose bead comprising reacting a modified ribonucleic acid of embodiment 17A with an azide-functionalized agarose bead.
20A. A kit comprising a compound, or salt thereof, of any of embodiments 1A-8A.
1B. A method for transcriptome-wide mapping of RNA binding sites in cells, comprising:
   a. Providing purified total RNA from cells;
   b. Combining the purified total RNA with a compound comprising a diazirine moiety and an alkyne moiety to form a mixture;
   c. Irradiating the mixture;
   d. Treating the irradiated mixture with a fluorescent dye comprising an azide or agarose comprising an azide under conditions wherein a triazolyl-molecule is formed;
   e. Evaluating the resulting triazolyl-molecules by gel electrophoresis or fluorescence imaging to identify the target and binding site within the target.
2B. A method for transcriptome-wide mapping of RNA binding sites in cells, comprising:
   a. Providing cells;
   b. Treating the cells with a compound comprising a diazirine moiety and an alkyne moiety to form a mixture;
   c. Irradiating the mixture;
   d. Treating the mixture with an agarose comprising an azide under conditions wherein a triazolyl-molecule is formed
   c. Harvesting from the mixture total RNA;
   f. Fragmenting the total RNA;
   g. Performing pulldown of triazolyl-bound RNA
   h. Evaluating the resulting triazolyl-molecules by gel electrophoresis imaging to identify the target gene and binding site within the target gene.
3B. The method of embodiment 1B or 2B, wherein the compound is a compound of Formula I:

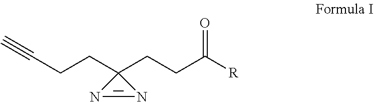

wherein
   R is selected from

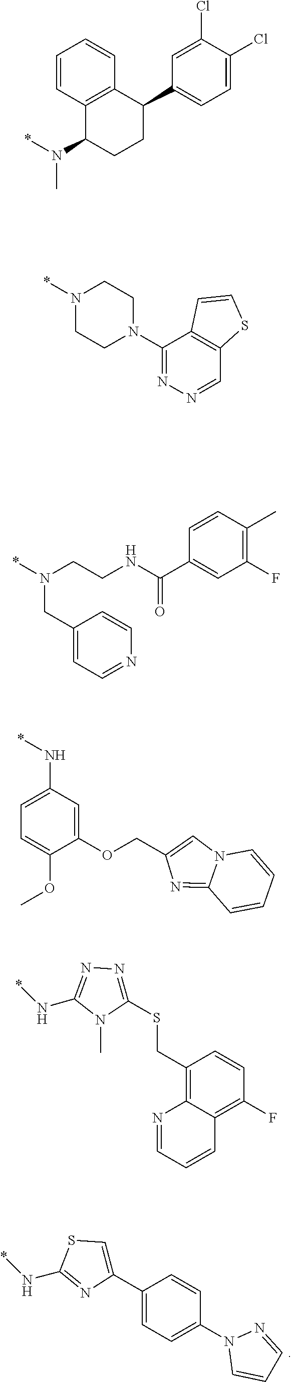 [SMILES: *N(C)[C@@H]1CC[C@H](c2ccc(Cl)c(Cl)c2)c2ccccc21 ; *N1CCN(c2nncc3sccc23)CC1 ; *N(CCNC(=O)c1ccc(C)c(F)c1)Cc1ccncc1 ; *Nc1ccc(OC)c(OCc2cn3ccccc3n2)c1 ; *Nc1nnc(SCc2ccc(F)c3cccnc23)n1C ; *Nc1nc(-c2ccc(-n3cccn3)cc2)cs1]

4B. The method of any of embodiments 1B-3B, wherein cell is a cancer cell.
5B. The method of any of embodiments 1B-3B, wherein cell is a breast cancer cell.
6B. The method of any of embodiments 1B-3B, wherein cell is a MDA-MB-231 triple negative breast cancer cell.
7B. The method of any of embodiments 1B-3B, wherein the RNA is QSOX1 RNA.
8B. The method of any of embodiments 1B-3B, wherein the RNA is QSOX1-a mRNA
9B. The method of any of embodiments 1B-3B, wherein the target is QSOX1.
10B. The method of any of embodiments 1B-3B, wherein the target is QSOX1-a.
11B. The method of any of embodiments 1B-3B, wherein the dye is tetramethylrhodamine (TAMRA) azide.

What is claimed is:

1. A method of transcriptome-wide mapping of RNA binding sites in a cell, comprising:
a. Providing purified total RNA from a cell;
b. Combining the purified total RNA with a compound comprising a diazirine moiety and an alkyne moiety to form a mixture;
c. Irradiating the mixture;
d. Treating the irradiated mixture with a fluorescent dye comprising an azide moiety or an agarose comprising an azide moiety under conditions wherein triazolyl-bound RNA is formed;
e Evaluating the resulting triazolyl-bound RNA to identify a target RNA.

2. A method of transcriptome-wide mapping of RNA binding sites in a cell, comprising:
a. Providing a cell;
b Treating the cell with a compound comprising a diazirine moiety and an alkyne moiety to form a mixture;
c. Irradiating the mixture;
d. Treating the irradiated mixture with a fluorescent dye comprising an azide moiety or an agarose comprising an azide moiety under conditions wherein triazolyl-bound RNA is formed; and
e. Evaluating the resulting triazolyl-bound RNA to identify a target RNA.

3. The method of any one of claim 1 or 2, further comprising:
f. Harvesting from the mixture total RNA comprising the triazolyl-bound RNA;
g Fragmenting the total RNA;
h. Performing pull-down of the triazolyl-bound RNA; and
i. Identifying a binding site within the target RNA.

4. The method of any one of claims 1-3, wherein the cell is a cancer cell.

5. The method of any one of claims 1-4, wherein the cell is a breast cancer cell.

6. The method of any one of claims 1-5, wherein the cell is an MDA-MB-231 triple negative breast cancer cell.

7. The method of any one of claims 1-6, wherein the compound is a compound of Formula (I): 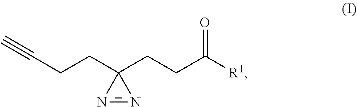
or a pharmaceutically acceptable salt thereof, wherein R¹ is selected from the group consisting of:

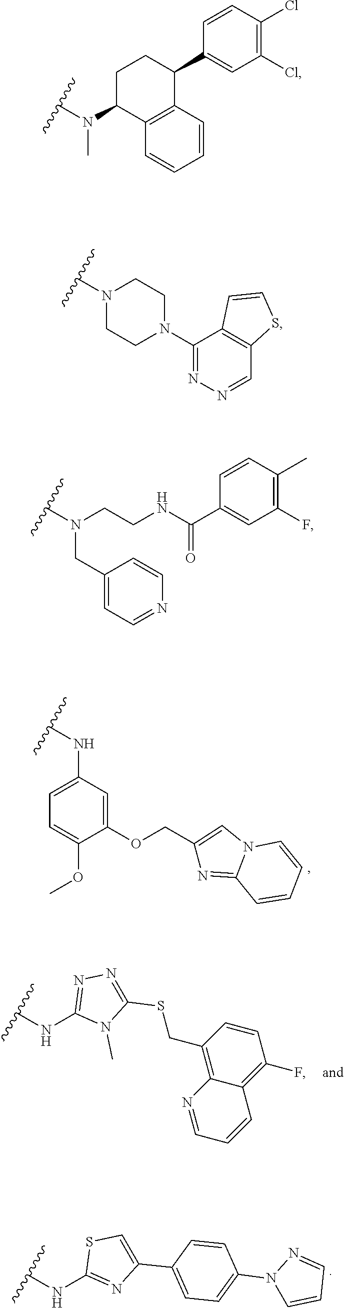 [SMILES: *N(C)[C@H]1CC[C@@H](c2ccc(Cl)c(Cl)c2)c2ccccc21 ; *N1CCN(c2nncc3sccc23)CC1 ; *N(CCNC(=O)c1ccc(C)c(F)c1)Cc1ccncc1 ; *Nc1ccc(OC)c(OCc2cn3ccccc3n2)c1 ; *Nc1nnc(SCc2ccc(F)c3cccnc23)n1C ; *Nc1nc(-c2ccc(-n3cccn3)cc2)cs1]

8. The method of any one of claims 1-7, wherein the mixture is incubated before irradiation.

9. The method of claim 8, wherein the incubation is for at least 30 minutes.

10. The method of any one of claim 8 or 9, wherein the incubation is for at least 16 hours.

11. The method of any one of claims 1-10, wherein the irradiation is for at least 10 minutes.

12. The method of any one of claims 1-11, wherein the irradiation is with ultraviolet light.

13. The method of any one of claims 1-12, wherein treating the irradiated mixture under conditions wherein triazolyl-bound RNA is formed comprises a copper (II) salt and a reducing agent.

14. The method of any one of claims 1-13, wherein treating the irradiated mixture under conditions wherein triazolyl-bound RNA is formed is for at least 3 hours.

15. The method of any one of claims 1-14, wherein evaluating the resulting triazolyl-bound RNA is by gel electrophoresis or fluorescence imaging.

16. The method of any one of claims 1-15, wherein evaluating the resulting triazolyl-bound RNA is by fluorescence imaging.

17. The method of any one of claims 1-16, wherein evaluating the resulting triazolyl-bound RNA comprises imaging the fluorescence of the fluorescent dye.

18. The method of any one of claims 1-17, wherein evaluating the resulting triazolyl-bound RNA comprises imaging tetramethylrhodamine (TAMRA) fluorescence.

19. The method of any one of claims 1-15, wherein evaluating the resulting triazolyl-bound RNA is by gel electrophoresis.

20. The method of any one of claim 1-15 or 19, further comprising treating the gel with an agent capable of staining the gel.

21. The method of any one of claim 1-15, 19, or 20, wherein the gel is stained with SYBR Green and/or Coomassie staining.

22. The method of any one of claims 1-21, wherein evaluating the resulting triazolyl-bound RNA comprises detecting a target signal at least 3-fold above a background signal.

23. The method of any one of claims 1-22, wherein evaluating the resulting triazolyl-bound RNA comprises identifying enrichment of the target RNA.

24. The method of any one of claims 1-23, wherein the method is selective for enrichment of the target RNA compared to DNA or proteins.

25. The method of any one of claims 1-24, wherein the compound comprises a moiety capable of binding RNA.

26. The method of any one of claims 1-25, wherein evaluating the resulting triazolyl-bound RNA comprises identifying a control target that non-specifically reacts with the diazirine moiety.

27. The method of any one of claims 1-26, further comprising using a control compound comprising a diazirine moiety and an alkyne moiety, wherein the control compound does not comprise a moiety capable of binding RNA, to identify a control target that non-specifically reacts with the diazirine moiety.

28. The method of any one of claims 1-27, wherein the RNA is QSOX1 mRNA.

29. The method of any one of claims 1-28, wherein the RNA is QSOX1-a mRNA.

30. The method of any one of claims 3-29, wherein fragmenting the total RNA comprises random fragmentation.

31. The method of any one of claims 3-30, wherein performing pull-down of triazolyl-bound RNA comprises selectively pulling-down fragmented RNA regions bound by the compound.

32. The method of any one of claims 3-31, wherein the concentration of the compound is sufficient for the pull-down of triazolyl-bound RNA to enrich the target RNA.

33. The method of claim 32, wherein the concentration of the compound is at least 5 UM (˜6-fold) or 20 μM (˜12-fold).

34. The method of any one of claims 3-33, wherein the pull-down of triazolyl-bound RNA is performed for a time sufficient to enrich the target RNA.

35. The method of any one of claims 3-34, wherein the pull-down of triazolyl-bound RNA is performed for at least 8 hours.

36. The method of any one of claims 3-35, wherein the pull-down of triazolyl-bound RNA is performed for at least 16 hours.

37. The method of any one of claims 3-36, wherein the method does not pull-down a protein capable of forming an mRNA-protein complex.

38. The method of any one of claims 3-37, wherein the method does not pull-down a protein produced from the target RNA.

39. A method of making a modified ribonucleic acid, or a pharmaceutically acceptable salt thereof, comprising reacting a ribonucleic acid with a compound of Formula (I): 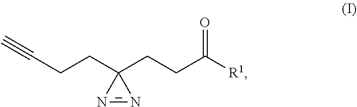
or a pharmaceutically acceptable salt thereof, wherein R¹ is selected from the group consisting of:

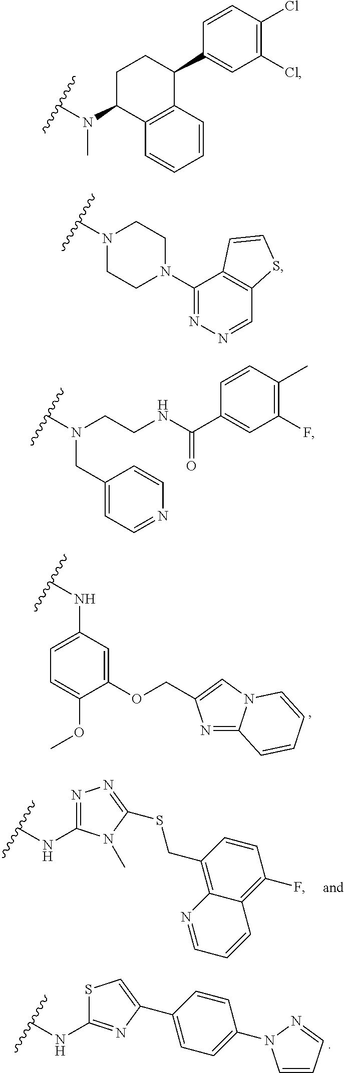

40. A modified ribonucleic acid, or a pharmaceutically acceptable salt thereof, made by the method of claim 39.

41. A method of making a fluorescent-tagged ribonucleic acid, or a pharmaceutically acceptable salt thereof, comprising reacting the modified ribonucleic acid of claim 40, or a pharmaceutically acceptable salt thereof, with a fluorescent dye comprising an azide moiety.

42. A method of making a modified agarose, comprising reacting the modified ribonucleic acid of claim 40, or a pharmaceutically acceptable salt thereof, with an agarose comprising an azide moiety.

43. A compound of Formula (I): 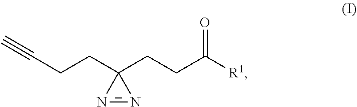
or a pharmaceutically acceptable salt thereof, wherein R¹ is selected from the group consisting of:

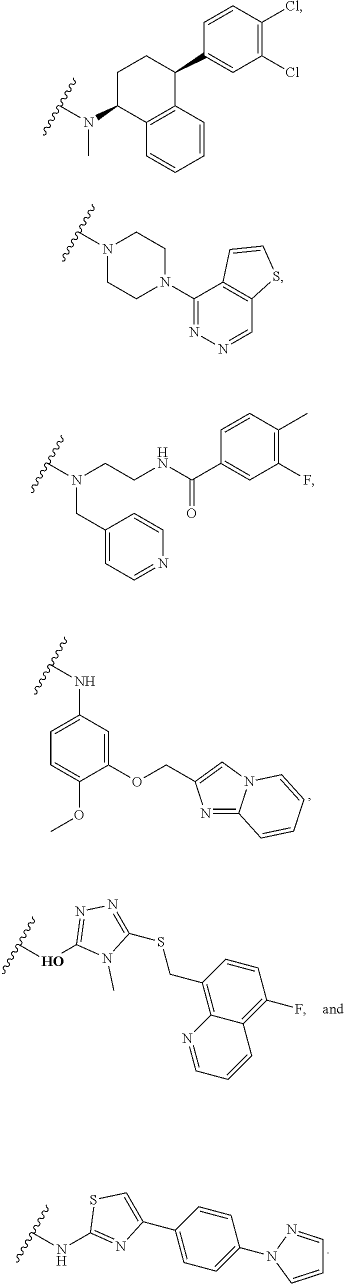 [SMILES: *N(C)[C@H]1CC[C@@H](c2ccc(Cl)c(Cl)c2)c2ccccc21 ; *N1CCN(c2nncc3sccc23)CC1 ; *N(CCNC(=O)c1ccc(C)c(F)c1)Cc1ccncc1 ; *Nc1ccc(OC)c(OCc2cn3ccccc3n2)c1 ; *Oc1nnc(SCc2ccc(F)c3cccnc23)n1C ; *Nc1nc(-c2ccc(-n3cccn3)cc2)cs1]

44. The compound of claim 43, wherein the compound of Formula (I) is of formula: 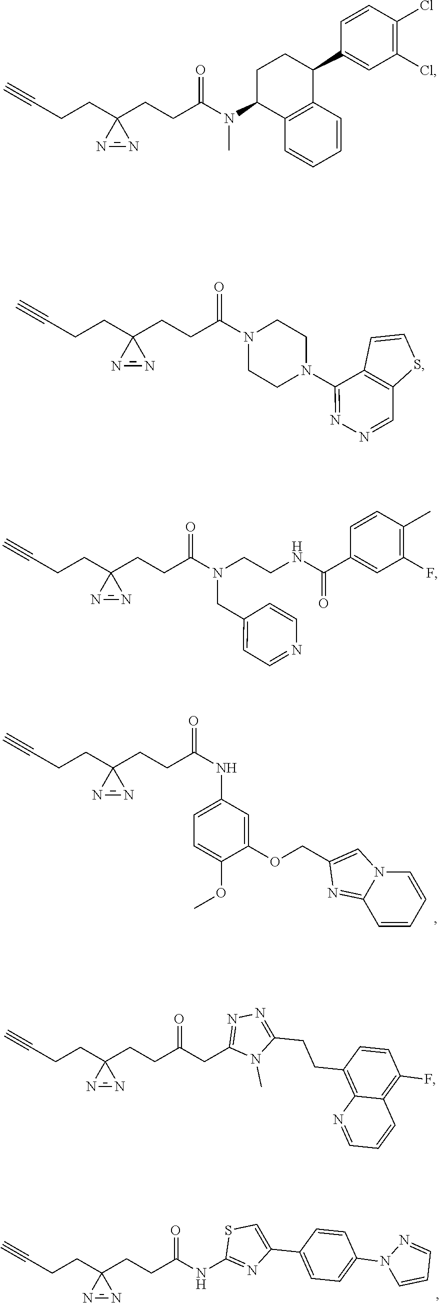 [SMILES: C#CCCC1(CCC(=O)N(C)[C@H]2CC[C@@H](c3ccc(Cl)c(Cl)c3)c3ccccc32)N=N1 ; C#CCCC1(CCC(=O)N2CCN(c3nncc4sccc34)CC2)N=N1 ; C#CCCC1(CCC(=O)N(CCNC(=O)c2ccc(C)c(F)c2)Cc2ccncc2)N=N1 ; C#CCCC1(CCC(=O)Nc2ccc(OC)c(OCc3cn4ccccc4n3)c2)N=N1 ; C#CCCC1(CCC(=O)Cc2nnc(CCc3ccc(F)c4cccnc34)n2C)N=N1 ; C#CCCC1(CCC(=O)Nc2nc(-c3ccc(-n4cccn4)cc3)cs2)N=N1]
or a pharmaceutically acceptable salt thereof.

45. The compound of any one of claim 43 or 44, wherein the compound of Formula (I) is of formula: 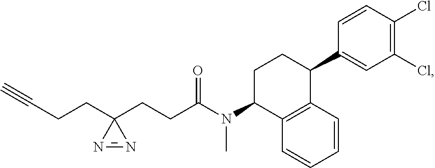 [SMILES: C#CCCC1(CCC(=O)N(C)[C@H]2CC[C@@H](c3ccc(Cl)c(Cl)c3)c3ccccc32)N=N1]
or a pharmaceutically acceptable salt thereof.

46. A compound of Formula (II):
B-L-R (II),
or a pharmaceutically acceptable salt thereof, wherein:
B is an RNA binder of formula: 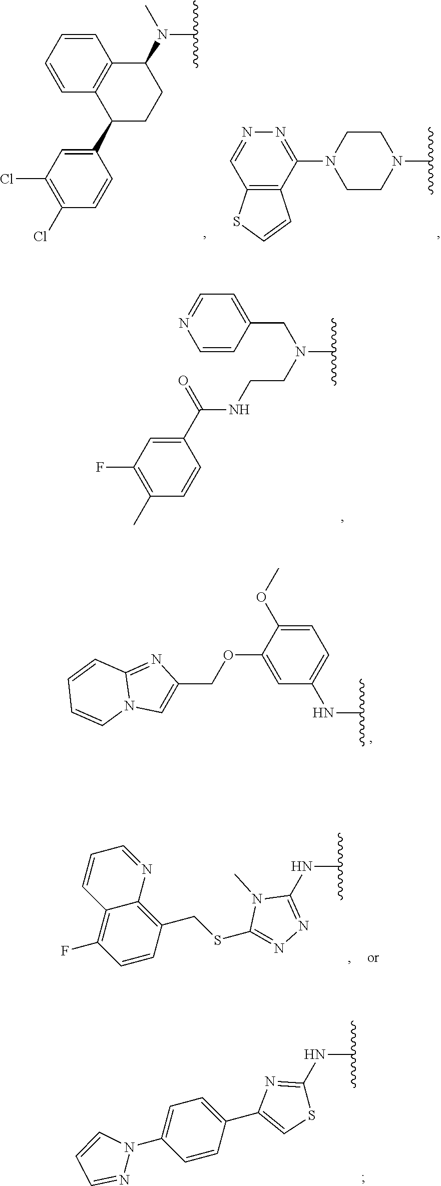 [SMILES: *N(C)[C@H]1CC[C@@H](c2ccc(Cl)c(Cl)c2)c2ccccc21 ; *N1CCN(c2nncc3sccc23)CC1 ; *N(CCNC(=O)c1ccc(C)c(F)c1)Cc1ccncc1 ; *Nc1ccc(OC)c(OCc2cn3ccccc3n2)c1 ; *Nc1nnc(SCc2ccc(F)c3cccnc23)n1C ; *Nc1nc(-c2ccc(-n3cccn3)cc2)cs1]
L is a linker, and
R is an RNase L recruiter.

47. The compound of claim 46, or a pharmaceutically acceptable salt thereof, wherein B is an RNA binder of Formula:

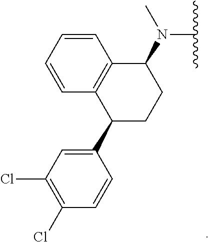

48. The compound of any one of claim 46 or 47, or a pharmaceutically acceptable salt thereof, wherein L is of Formula (III): 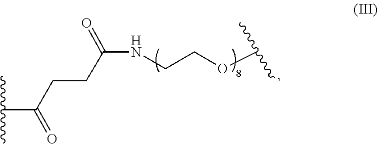
wherein n is 0, 1, 2, 3, 4, 5, 6, 7, 8, 9, or 10.

49. The compound of any one of claims 46-48, or a pharmaceutically acceptable salt thereof, wherein L is of formula:

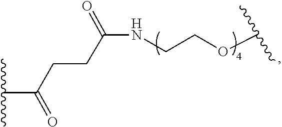

50. The compound of any one of claims 46-49, or a pharmaceutically acceptable salt thereof, wherein R is an RNAse L recruiter of Formulae (IV-a) or (IV-b):

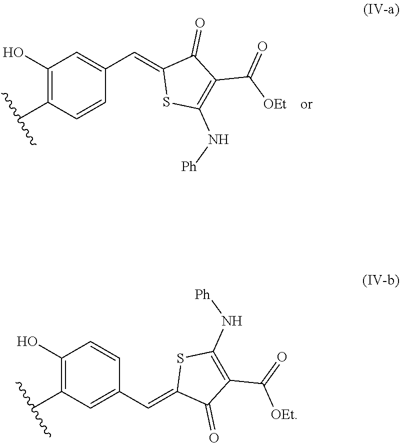 [SMILES: *c1ccc(/C=C2\SC(Nc3ccccc3)=C(C(=O)OCC)C2=O)cc1O ; *c1cc(/C=C2\SC(Nc3ccccc3)=C(C(=O)OCC)C2=O)ccc1O]

51. The compound of any one of claims 46-50, or a pharmaceutically acceptable salt thereof, wherein R is an RNAse L recruiter of Formula (IV-a):

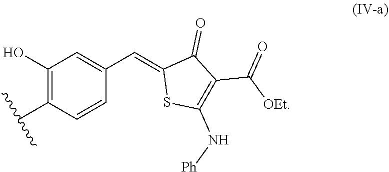 [SMILES: *c1ccc(/C=C2\SC(Nc3ccccc3)=C(C(=O)OCC)C2=O)cc1O]

52. The compound of any one of claims 46-51, or a pharmaceutically acceptable salt thereof, wherein the compound is of Formula (II-a): 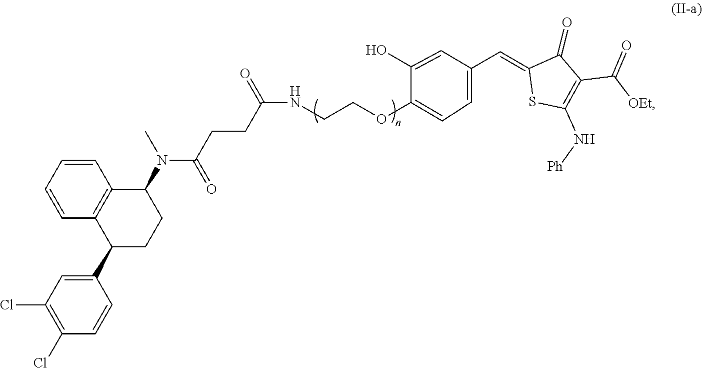
wherein n is 0, 1, 2, 3, 4, 5, 6, 7, 8, 9, or 10.

53. The compound of any one of claims 46-52, or a pharmaceutically acceptable salt thereof, wherein the compound is of formula:

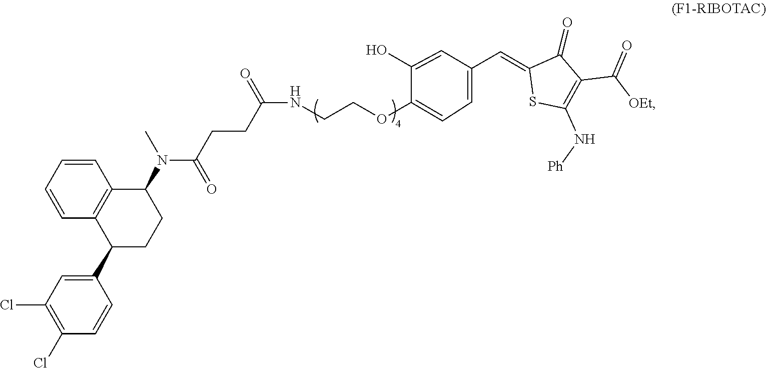

54. The compound of any one of claims 46-50, or a pharmaceutically acceptable salt thereof, wherein R is an RNAse L recruiter of Formula (IV-b):

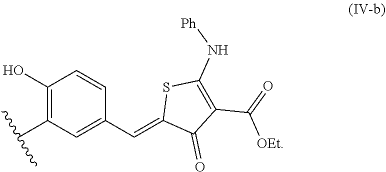 [SMILES: *c1cc(/C=C2\SC(Nc3ccccc3)=C(C(=O)OCC)C2=O)ccc1O]

55. The compound of any one of claim 46-50 or 54, or a pharmaceutically acceptable salt thereof, wherein the compound is of Formula (II-b): 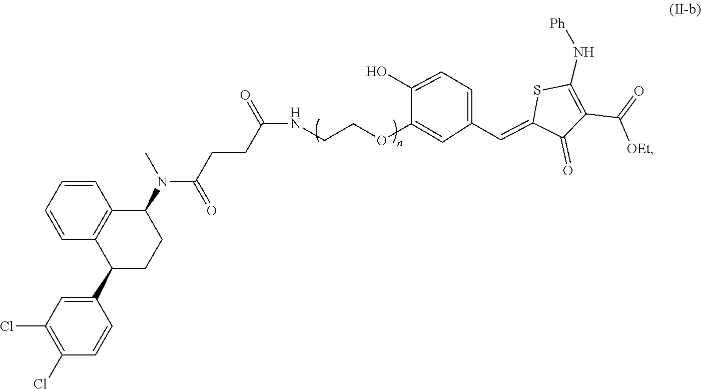
wherein n is 0, 1, 2, 3, 4, 5, 6, 7, 8, 9, or 10.

56. The compound of any one of claim 46-50, 54, or 55, or a pharmaceutically acceptable salt thereof, wherein the compound is of formula:

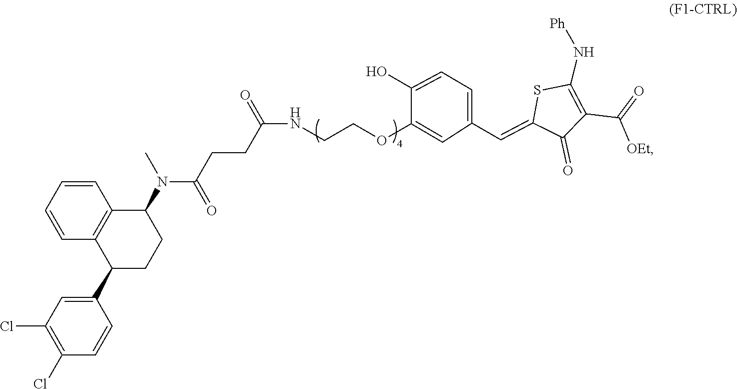

57. A composition comprising the compound of any one of claims 46-56, or a pharmaceutically acceptable salt thereof, and an excipient.

58. A method of binding RNase L in a subject in need thereof or in a cell, tissue, or biological sample, comprising administering to the subject in need thereof or contacting the cell, tissue, or biological sample with an effective amount of:
a compound of any one of claims 46-56, or a pharmaceutically acceptable salt thereof; or
the composition of claim 57.

59. The method of claim 58, wherein binding RNase L comprises activating RNase L.

60. The method of any one of claim 58 or 59, wherein binding RNase L comprises inducing RNase L dimerization.

61. The method of any one of claims 58-60, further comprising modulating QSOX1-a mRNA.

62. The method of any one of claims 58-61, further comprising degrading QSOX1-a mRNA.

63. A method of modulating QSOX1 mRNA in a subject in need thereof or in a cell, tissue, or biological sample, comprising administering to the subject in need thereof or contacting the cell, tissue, or biological sample with an effective amount of:
a compound of any one of claims 46-56, or a pharmaceutically acceptable salt thereof, or the composition of claim 57.

64. The method of claim 63, wherein the QSOX1 mRNA is QSOX1-a mRNA.

65. A method of degrading a QSOX1 mRNA isoform in a subject in need thereof or in a cell, tissue, or biological sample, comprising administering to the subject in need thereof or contacting the cell, tissue, or biological sample with an effective amount of:
a compound of any one of claims 46-56, or a pharmaceutically acceptable salt thereof, or the composition of claim 57.

66. The method of claim 65, wherein the QSOX1 mRNA isoform is QSOX1-a.

67. A method of inhibiting cell proliferation or promoting apoptosis in a subject in need thereof or in a cell, tissue, or biological sample, comprising administering to the subject in need thereof or contacting the cell, tissue, or biological sample with an effective amount of:
a compound of any one of claims 46-56, or a pharmaceutically acceptable salt thereof; or
the composition of claim 57.

68. The method of any one of claims 58-67, further comprising reducing an amount of QSOX1 protein.

69. The method of claim 68, wherein the QSOX1 protein is QSOX1-a.

70. A method of treating or preventing a disease in a subject in need thereof, comprising administering to the subject in need thereof an effective amount of:
a compound of any one of claims 46-56, or a pharmaceutically acceptable salt thereof, or the composition of claim 57.

71. The compound of any one of claims 46-56, or a pharmaceutically acceptable salt thereof, or the composition of claim 57, for use in treating a disease in a subject in need thereof.

72. The compound of any one of claims 46-56, or a pharmaceutically acceptable salt thereof, or the composition of claim 57, for use in the manufacture of a medicament for treatment of a disease in a subject in need thereof.

73. The method, compound for use, pharmaceutically acceptable salt thereof for use, or composition for use of any one of claims 70-72, wherein the disease is associated with QSOX1 mRNA.

74. The method, compound for use, pharmaceutically acceptable salt thereof for use, or composition for use of any one of claims 70-73, wherein the disease is a proliferative disease.

75. The method, compound for use, pharmaceutically acceptable salt thereof for use, or composition for use of claim 74, wherein the proliferative disease is cancer.

76. The method, compound for use, pharmaceutically acceptable salt thereof for use, or composition for use of claim 75, wherein the cancer is breast cancer.

77. The method, compound for use, pharmaceutically acceptable salt thereof for use, or composition for use of any one of claim 75 or 76, wherein the cancer is triple negative breast cancer.

78. A method of preparing a compound of Formulae (II-a) or (II-b): 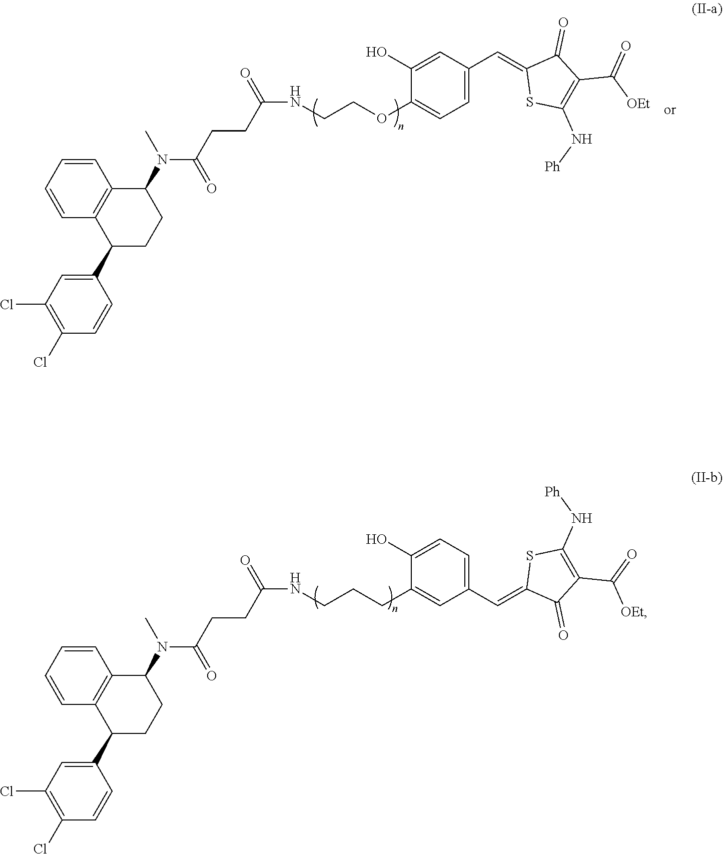
or a pharmaceutically acceptable salt thereof, comprising reacting a compound of Formulae (V-a) or (V-b): 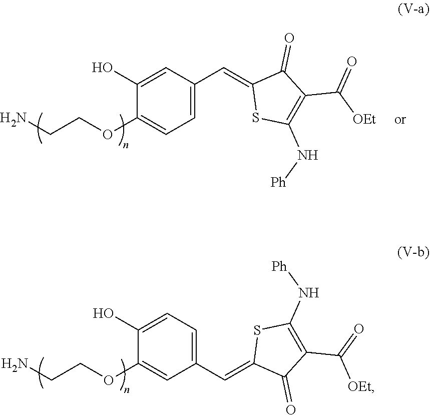
or a salt thereof, with a compound of Formula (VI): 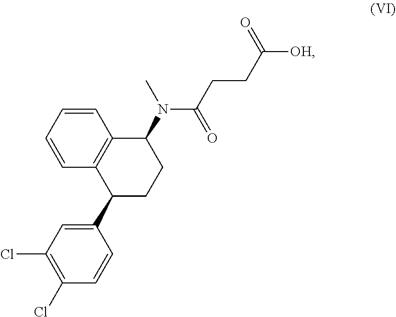 [SMILES: CN(C(=O)CCC(=O)O)[C@H]1CC[C@@H](c2ccc(Cl)c(Cl)c2)c2ccccc21]
or a salt thereof, wherein n is 0, 1, 2, 3, 4, 5, 6, 7, 8, 9, or 10.

79. The method of claim 78, further comprising alkylating a compound of Formula (VII): 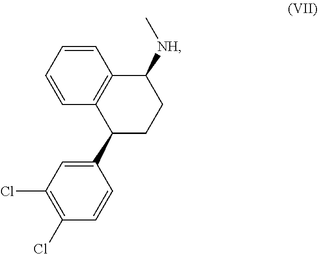 [SMILES: CN[C@H]1CC[C@@H](c2ccc(Cl)c(Cl)c2)c2ccccc21]
or a salt thereof, to provide the compound of Formula (VI), or salt thereof.

80. The method of any one of claim 78 or 79, further comprising alkylating a compound of Formulae (VIII-a) or (VII-b): 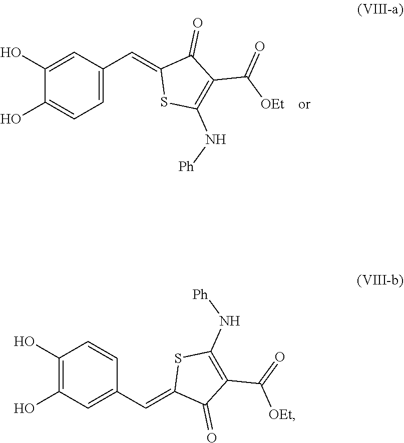 [SMILES: CCOC(=O)C1=C(Nc2ccccc2)S/C(=C\c2ccc(O)c(O)c2)C1=O ; CCOC(=O)C1=C(Nc2ccccc2)S/C(=C\c2ccc(O)c(O)c2)C1=O]
or a salt thereof, to provide the compound of Formulae (V-a) or (V-b): 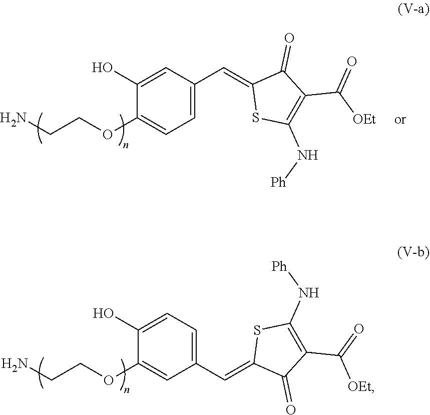
or a salt thereof.

81. A kit comprising the compound of any one of claims 46-56, or a pharmaceutically acceptable salt thereof, or the composition of claim 57, and instructions for its use.


SEQUENCE LISTING (16 sequences)

SEQ ID NO: 1 - DNA, 22 bp, Synthetic construct
  source 1..22: mol_type = other DNA
gtgcttgcgtgtggtggtgagc

SEQ ID NO: 2 - DNA, 24 bp, Synthetic construct
  source 1..24: mol_type = other DNA
cagcgggtcggaaggcgaatagag

SEQ ID NO: 3 - DNA, 23 bp, Synthetic construct
  source 1..23: mol_type = other DNA
tatggcgtcgctcaccgtgaagg

SEQ ID NO: 4 - DNA, 21 bp, Synthetic construct
  source 1..21: mol_type = other DNA
cagcctcggcttccgcctcag

SEQ ID NO: 5 - DNA, 20 bp, Synthetic construct
  source 1..20: mol_type = other DNA
gtaacccgttgaaccccatt

SEQ ID NO: 6 - DNA, 20 bp, Synthetic construct
  source 1..20: mol_type = other DNA
ccatccaatcggtagtagcg

SEQ ID NO: 7 - DNA, 16 bp, synthetic construct
  source 1..16: mol_type = other DNA
  misc_feature 1..3: note = RNA
  misc_feature 4..16: note = DNA
tgttggtctcctcagc

SEQ ID NO: 8 - RNA, 10 nt, Synthetic construct
  source 1..10: mol_type = other RNA
gtgagggtca

SEQ ID NO: 9 - RNA, 31 nt, Synthetic construct
  source 1..31: mol_type = other RNA
ggtgcttgcgtgtggtggtgagcgcagcgcc

SEQ ID NO: 10 - RNA, 32 nt, Synthetic construct
  source 1..32: mol_type = other RNA
ggtgcttgcgtgtggtggtgagcgcaagcgcc

SEQ ID NO: 11 - RNA, 38 nt, Synthetic construct
  source 1..38: mol_type = other RNA
ggtgcttgcgtgtggtggtgagcgcagcgccgaggatg

SEQ ID NO: 12 - DNA, 55 bp, Synthetic construct
  source 1..55: mol_type = other DNA
atccggtgcttgcgtgtggtggtgagcgcagcgccgaggatgaggaggtgcaaca

SEQ ID NO: 13 - DNA, 55 bp, Synthetic construct
  source 1..55: mol_type = other DNA
tgttgcacctcctcatcctcggcgctgcgctcaccaccacacgcaagcaccggat

SEQ ID NO: 14 - DNA, 28 bp, Synthetic construct
  source 1..28: mol_type = other DNA
cagcgccgaggatgaggaggtgcaacca

SEQ ID NO: 15 - DNA, 27 bp, Synthetic construct
  source 1..27: mol_type = other DNA
tgttgcacctcctcatcctcggcgctg

SEQ ID NO: 16 - RNA, 49 nt, Synthetic construct
  source 1..49: mol_type = other RNA
gcgtcgctcaccgtgaaggcctaccttctgggcaaggaggacgcggcgc